(19) United States
(12) Patent Application Publication
(10) Pub. No.: US 2022/0135620 A1
(43) Pub. Date: May 5, 2022

(54) PURIFICATION PLATFORMS FOR OBTAINING PHARMACEUTICAL COMPOSITIONS HAVING A REDUCED HYDROLYTIC ENZYME ACTIVITY RATE

(71) Applicant: Genentech, Inc., South San Francisco, CA (US)

(72) Inventors: Alex SEAY, South San Francisco, CA (US); Marc WONG, South San Francisco, CA (US); Stephen WOON, South San Francisco, CA (US); Michael LEE, South San Francisco, CA (US); Stefanie KHOO, South San Francisco, CA (US); William O'DWYER, South San Francisco, CA (US); Eileen DUENAS, South San Francisco, CA (US); Yinges YIGZAW, South San Francisco, CA (US); Amy LIM, South San Francisco, CA (US)

(21) Appl. No.: 17/513,710

(22) Filed: Oct. 28, 2021

Related U.S. Application Data
(60) Provisional application No. 63/108,194, filed on Oct. 30, 2020.

(51) Int. Cl. C07K 1/36; C07K 16/00; C07K 1/34; C07K 1/18; B01D 15/36; B01D 15/38; B01D 15/32; B01D 15/42; B01D 61/14; B01D 61/16; B01D 39/20; B01D 39/18; B01D 39/16

(52) U.S. Cl. CPC C07K 1/36; B01D 2239/0414; C07K 1/34; C07K 1/18; B01D 15/363; B01D 15/362; B01D 15/3847; B01D 15/327; B01D 15/3809; B01D 15/424; B01D 61/145; B01D 61/16; B01D 39/2068; B01D 39/18; B01D 39/1615; B01D 39/1623; B01D 2315/16; B01D 2311/2623; B01D 2311/2649; B01D 2311/04; B01D 2311/2626; C07K 16/00

(57) ABSTRACT

The present disclosure provides purification platforms comprising a depth filter step and/or a hydrophobic interaction chromatography (HIC) step and/or a MM-HIC/IEX chromatography step, and are useful for providing a method of reducing a hydrolytic enzyme activity rate of a composition obtained from said purification platforms. Also disclosed herein are methods of using the purification platforms described herein and compositions obtained therefrom, such as pharmaceutical compositions.

### CROSS-REFERENCE TO RELATED APPLICATIONS

This application claims priority benefit of U.S. Provisional Application No. 63/108,194, filed on Oct. 30, 2020, the contents of which are incorporated herein by reference in its entirety.

### TECHNICAL FIELD

The present disclosure is directed to purification platforms for obtaining compositions, such as pharmaceutical compositions, having a reduced hydrolytic enzyme activity rate. Also disclosed herein are methods of using the purification platforms described herein and compositions obtained therefrom.

### BACKGROUND

Biotherapeutic products, such as antibodies, produced from host cell cultures require purification to remove host cell proteins and other impurities that may impact, e.g., product quality and therapeutic efficacy. Current purification methods may not remove all host cell proteins and impurities, including host cell hydrolytic enzymes. Host cell proteins and impurities remaining with the purification target can thus impact the purification target itself as well as other additives, e.g., components added for formulation purposes, such as surfactants. Accordingly, there is a need for improved approaches for purifying products produced from host cell cultures for pharmaceutical use.

All references cited herein, including patent applications and publications, are incorporated herein by reference in their entirety.

### BRIEF SUMMARY

In some aspects, provided is a method of reducing a hydrolytic enzyme activity rate of a composition obtained from a purification platform, the method comprising subjecting a sample to the purification platform comprising: a capture step; one or more ion exchange (IEX) chromatography steps; and a depth filtration step, thereby reducing the hydrolytic enzyme activity rate of the composition as compared to purification of the sample without the depth filtration step.

In some embodiments, each of the one or more IEX chromatography steps is selected from the group consisting of: an anion exchange (AEX) chromatography step, a cation exchange (CEX) chromatography step, and a multimodal ion exchange (MMIEX) chromatography step. In some embodiments, the MMIEX chromatography step comprises a multimodal cation exchange/anion exchange (MM-AEX/CEX) chromatography step.

In some embodiments, the method further comprises a virus filtration step.

In some embodiments, the method further comprises an ultrafiltration/diafiltration (UF/DF) step.

In some embodiments, the purification platform comprises, in order: the capture step; the CEX chromatography step; the AEX chromatography step; the depth filtration step; the virus filtration step; and the UF/DF step.

In some embodiments, the depth filtration step comprises processing via a depth filter, and wherein the depth filter is a X0SP depth filter, a C0SP depth filter, a D0SP depth filter, or an EMPHAZE™ depth filter.

In some embodiments, the method further comprises a HIC step comprising processing via Sartobind® phenyl.

In other aspects, provided is a method of reducing a hydrolytic enzyme activity rate of a composition obtained from a purification platform, the method comprising subjecting a sample to the purification platform comprising: a capture step; a multimodal hydrophobic interaction/ion exchange (MM-HIC/IEX) chromatography step; and a hydrophobic interaction chromatography (HIC) step, thereby reducing the hydrolytic enzyme activity rate of the composition as compared to purification of the sample without the HIC step and/or the MM-HIC/IEX chromatography step.

In some embodiments, the MM-HIC/IEX chromatography step comprises processing via a MM-HIC/IEX chromatography medium and is performed at a pH of about 5.5 to about 8. In some embodiments, the MM-HIC/IEX chromatography step is a multimodal hydrophobic interaction/anion exchange (MM-HIC/AEX) chromatography step. In some embodiments, the MM-HIC/AEX chromatography step comprises processing via Capto™ Adhere or Capto™ Adhere ImpRes. In some embodiments, the MM-HIC/IEX chromatography step is a multimodal hydrophobic interaction/cation exchange (MM-HIC/CEX) chromatography step. In some embodiments, the MM-HIC/CEX chromatography step comprises Capto™ MMC or Capto™ MMC ImpRes.

In some embodiments, the purification platform comprises, in order: the capture step; the MM-HIC/IEX chromatography step; and the HIC step.

In some embodiments, the method further comprises a virus filtration step.

In some embodiments, the method further comprises an ultrafiltration/diafiltration (UF/DF) step.

In some embodiments, the purification platform comprises, in order: the capture step; the MM-HIC/AEX chromatography step; the HIC step; the virus filtration step; and the UF/DF step.

In some embodiments, the method further comprises a depth filtration step.

In some embodiments, the purification platform comprises, in order: the capture step; the depth filtration step; the MM-HIC/AEX chromatography step; and the HIC step. In some embodiments, the depth filtration step comprises processing via a depth filter, and wherein the depth filter is a X0SP depth filter. In some embodiments, the depth filtration step comprises processing via a depth filter, and the depth filter is an EMPHAZE™ depth filter.

In some embodiments, the depth filter is used as a load filter in conjunction with the MM-HIC/AEX chromatography step.

In some embodiments, the MM-HIC/AEX chromatography step comprises processing via Capto™ Adhere or Capto™ Adhere ImpRes.

In some embodiments, the purification platform comprises, in order: the capture step; the MM-HIC/AEX chromatography step; the depth filtration step; and the HIC step. In some embodiments, the depth filtration step comprises processing via a depth filter, and wherein the depth filter is a X0SP depth filter, a C0SP depth filter, or a D0SP depth filter. In some embodiments, the depth filtration step comprises processing via a depth filter, and the depth filter is an EMPHAZE™ depth filter. In some embodiments, the MM-HIC/AEX chromatography step comprises processing via Capto™ Adhere or Capto™ Adhere ImpRes.

In other aspects, provided is a method of reducing a hydrolytic enzyme activity rate of a composition obtained from a purification platform, the method comprising subjecting a sample to the purification platform comprising: a capture step; one or more ion exchange (IEX) chromatography steps; and a hydrophobic interaction chromatography (HIC) step, thereby reducing the hydrolytic enzyme activity rate of the composition as compared to purification of the sample without the HIC step.

In some embodiments, the one or more IEX chromatography steps is a cation exchange (CEX) chromatography step.

In some embodiments, the method further comprises a virus filtration step.

In some embodiments, the method further comprises an ultrafiltration/diafiltration (UF/DF) step.

In some embodiments, the method further comprises a depth filtration step performed at any stage prior to the UF/DF step.

In some embodiments, the purification platform comprises, in order: the capture step; the CEX chromatography step; the HIC step; the virus filtration step; and the UF/DF step.

In other aspects, provided is a method of reducing a hydrolytic enzyme activity rate of a composition obtained from a purification platform, the method comprising subjecting a sample to the purification platform comprising: one or more ion exchange (IEX) chromatography steps; a hydrophobic interaction chromatography (HIC) step; and a depth filtration step, thereby reducing the hydrolytic enzyme activity rate of the composition as compared to purification of the sample without the HIC step or the depth filtration step. In some embodiments, the reduction is as compared to purification of the sample without the HIC and the depth filtration step.

In some embodiments, each of the one or more IEX chromatography steps is selected from the group consisting of: an anion exchange (AEX) chromatography step, a cation exchange (CEX) chromatography step, and a multimodal ion exchange (MMIEX) chromatography step. In some embodiments, the MMIEX chromatography step comprises a multimodal cation exchange/anion exchange (MM-AEX/CEX) chromatography step.

In some embodiments, the method further comprises an ultrafiltration/diafiltration (UF/DF) step.

In some embodiments, the purification platform comprises, in order: the CEX chromatography step; the HIC step; the MMIEX chromatography step; the AEX chromatography step; the depth filter step; and the UF/DF step.

In other aspects, provided is a method of reducing a hydrolytic enzyme activity rate of a composition obtained from a purification platform, the method comprising subjecting a sample to the purification platform comprising: a capture step; one or more ion exchange (IEX) chromatography steps; a multimodal hydrophobic interaction/ion exchange (MM-HIC/IEX) chromatography steps; and one or both of: a hydrophobic interaction chromatography (HIC) step; and a depth filtration step, thereby reducing the hydrolytic enzyme activity rate of the composition as compared to purification of the sample without the HIC step or the depth filtration step.

In some embodiments, the reduction is as compared to purification of the sample without the HIC and the depth filtration step.

In some embodiments, the method further comprises a virus filtration step.

In some embodiments, the method further comprises an ultrafiltration/diafiltration (UF/DF) step.

In some embodiments, the depth filtration step is performed as a load filter for the MM-HIC/IEX chromatography step, as a load filter for the HIC step, or following the HIC step.

In some embodiments, each of the one or more IEX chromatography steps is selected from the group consisting of: a cation exchange (CEX) chromatography step, an anion exchange (AEX) chromatography step, and a multimodal ion exchange (MMIEX) chromatography step.

In some embodiments, the MM-HIC/IEX chromatography step is a multimodal hydrophobic interaction/anion exchange (MM-HIC/AEX) chromatography step. In some embodiments, the MM-HIC/AEX chromatography step comprises processing via Capto™ Adhere or Capto™ Adhere ImpRes.

In some embodiments, the capture step comprises processing via affinity chromatography. In some embodiments, the capture step is performed in a bind-and-elute mode. In some embodiments, the affinity chromatography is selected from the group consisting of a protein A chromatography, a protein G chromatography, a protein A/G chromatography, a FcXL chromatography, a protein XL chromatography, a kappa chromatography, and a kappaXL chromatography.

In some embodiments, the depth filtration step comprises processing via a depth filter. In some embodiments, the depth filter is used as a load filter. In some embodiments, the depth filter comprises a substrate comprising one or more of a diatomaceous earth composition, a silica composition, a cellulose fiber, a polymeric fiber, a cohesive resin, and an ash composition. In some embodiments, at least a portion of the substrate of the depth filter comprises a surface modification. In some embodiments, the surface modification is one or more of a quaternary amine surface modification (such as a quaternary ammonium, Q, functionality), a guanidinium surface modification, a cationic surface modification, and an anionic surface modification. In some embodiments, the depth filter is selected from the group consisting of: a X0SP depth filter, a D0SP depth filter, a C0SP depth filter, an EMPHAZE™ depth filter, a PDD1 depth filter, a PDE1 depth filter, a PDH5 depth filter, a ZETA PLUS™ 120ZA depth filter, a ZETA PLUS™ 120ZB depth filter, a ZETA PLUS™ DELI depth filter, a ZETA PLUS™ DELP depth filter, and a Polisher ST (salt tolerant) depth filter. In some embodiments, the depth filter is the X0SP depth filter, the D0SP depth filter, or the C0SP depth filter, and wherein the processing via the depth filter is performed at a pH of about 4.5 to about 8. In some embodiments, the depth filter is the EMPHAZE™ depth filter, and wherein processing via the depth filter is performed at a pH of about 7 to about 9.5. In some embodiments, the depth filter is the Polisher ST depth filter, and wherein processing via the depth filter is performed at a pH of about 5 to about 9.

In some embodiments, the HIC step comprises processing via a HIC membrane or a HIC column. In some embodiments, processing via the HIC membrane or the HIC column is performed using low salt concentrations. In some embodiments, processing via the HIC membrane or the HIC column is performed in flow-through mode. In some embodiments, the HIC membrane or HIC column comprises a substrate comprising one or more of an ether group, an ethyl group, a propyl group, an isopropyl group, a butyl group, a hexyl group, an octyl group, and a phenyl group. In some embodiments, the HIC membrane or the HIC column is selected from the group consisting of Bakerbond WP HI-Propyl™, Phenyl Sepharose® Fast Flow (Phenyl-SFF), Phenyl Sepharose® Fast Flow Hi-sub (Phenyl-SFF HS), Toyopearl® Hexyl-650C, Toyopearl® Hexyl-650M, Toyopearl® Hexyl-650S, Poros™ Benzyl Ultra, and Sartobind® phenyl. In some embodiments, processing via the HIC membrane or the HIC column is performed at a pH of about 4.5 to about 7.

In some embodiments, each of the one or more IEX chromatography steps comprises processing via an IEX chromatography membrane or an IEX chromatography column. In some embodiments, the IEX chromatography membrane or the IEX chromatography column is selected from the group consisting of: SPSFF, QSFF, SPXL, Streamline™ SPXL, ABx™, Poros™ XS, Poros™ 50HS, DEAE, DMAE, TMAE, QAE, and MEP-Hypercel™.

In some embodiments, the purification platform is for purification of a target from the sample, and wherein the sample comprises the target and one or more host cell impurities. In some embodiments, the target comprises a polypeptide. In some embodiments, the target is an antibody moiety. In some embodiments, the antibody moiety is a monoclonal antibody. In some embodiments, the antibody moiety is a human, humanized, or chimeric antibody. In some embodiments, the antibody moiety is selected from the group consisting of: an anti-TAU antibody, an anti-TGFβ3 antibody, an anti-VEGF-A antibody, an anti-CD20 antibody, an anti-CD40 antibody, an anti-HER2 antibody, an anti-IL6 antibody, an anti-IgE antibody, an anti-IL13 antibody, an anti-TIGIT antibody, an anti-PD-L1 antibody, an anti-VEGF-A/ANG2 antibody, an anti-CD79b antibody, an anti-ST2 antibody, an anti-factor D antibody, an anti-factor IX antibody, an anti-factor X antibody, an anti-abeta antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, a FAP-IL2v fusion protein, and a TYRP1 TCB antibody. In some embodiments, the antibody moiety is selected from the group consisting of: ocrelizumab, pertuzumab, ranibizumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, omalizumab, ranibizumab, emicizumab, selicrelumab, prasinezumab, R06874281, and R07122290.

In some embodiments, the one or more host cell impurities comprises a host cell protein. In some embodiments, the host cell protein is a hydrolytic enzyme. In some embodiments, the hydrolytic enzyme is a lipase, an esterase, a thioesterase, a phospholipase, carboxylesterase, hydrolase, cutinase, or a ceramidase.

In some embodiments, the sample comprises a host cell or components originating therefrom. In some embodiments, the sample is, or is derived from, a cell culture sample. In some embodiments, the cell culture sample comprises a host cell, and wherein the host cell is a Chinese hamster ovary (CHO) cell or an E. coli cell.

In some embodiments, the method further comprises a sample processing step.

In some embodiments, the reduction in the hydrolytic enzyme activity rate is at least about 20%.

In some embodiments, the method further comprises determining the hydrolytic enzyme activity rate of the composition.

In some embodiments, the method further comprises determining the level of one or more hydrolytic enzymes in the composition.

In some embodiments, the composition comprises a polysorbate. In some embodiments, the polysorbate is selected from the group consisting of polysorbate 20, polysorbate 40, polysorbate 60, and polysorbate 80.

In other aspects, provided is a pharmaceutical composition obtained from a method described herein.

In other aspects, provided is a formulated antibody moiety composition comprising an antibody moiety and a polysorbate, wherein the composition has a reduced rate of polysorbate hydrolysis, wherein the shelf-life of the composition is more than 12 months.

In other aspects, provided is a formulated antibody moiety composition comprising an antibody moiety and a polysorbate, wherein the composition has a reduced rate of polysorbate hydrolysis activity, wherein the shelf-life of the composition is extended compared to the shelf-life indicated in documents filed with a health authority related to the formulated antibody moiety composition, wherein the shelf-life is extended by at least 3 months compared to the shelf-life indicated in said documents.

In some embodiments, the rate of polysorbate hydrolysis is reduced by at least about 20%.

In other aspects, provided is a formulated antibody moiety composition comprising an antibody moiety, wherein the formulated antibody moiety composition has a reduced degradation of polysorbate, wherein the degradation is reduced by at least about 20% compared to the degradation indicated in documents filed with a health authority related to the formulated antibody moiety composition.

In other aspects, provided is a formulated antibody moiety composition comprising an antibody moiety and a polysorbate, wherein the polysorbate is degraded during storage of the liquid composition by 50% or less per year.

In some embodiments, the antibody moiety is a monoclonal antibody. In some embodiments, the antibody moiety is a human, humanized, or chimeric antibody. In some embodiments, the antibody is selected from the group consisting of an anti-TAU antibody, an anti-TGFβ3 antibody, an anti-VEGF-A antibody, an anti-CD20 antibody, an anti-CD40 antibody, an anti-HER2 antibody, an anti-IL6 antibody, an anti-IgE antibody, an anti-IL13 antibody, an anti-TIGIT antibody, an anti-PD-L1 antibody, an anti-VEGF-A/ANG2 antibody, an anti-CD79b antibody, an anti-ST2 antibody, an anti-factor D antibody, an anti-factor IX antibody, an anti-factor X antibody, an anti-abeta antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, a FAP-IL2v fusion protein, and a TYRP1 TCB antibody. In some embodiments, the antibody moiety is selected from the group consisting of ocrelizumab, pertuzumab, ranibizumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, omalizumab ranibizumab, emicizumab, selicrelumab, prasinezumab, RO6874281, and RO7122290.

In some embodiments, the polysorbate is selected from the group consisting of polysorbate 20, polysorbate 40, polysorbate 60, and polysorbate 80.

Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### BRIEF DESCRIPTION OF THE DRAWINGS

FIGS. 1A-1E show schematics of exemplary purification platforms described herein.

FIG. 2 shows a bar graph of the hydrolytic activity (as normalized to the control) of compositions obtained from exemplary purification platforms. The hydrolytic activity rates were measured using a FAMS assay.

FIG. 3 shows a bar graph of the hydrolytic activity (as normalized to the control) of compositions obtained from exemplary purification platforms. The hydrolytic activity rates were measured using a FAMS assay.

FIG. 4 shows a bar graph of the hydrolytic activity (as normalized to the control) of compositions obtained from exemplary purification platforms. The hydrolytic activity rates were measured using a FAMS assay.

FIG. 5 shows a bar graph of the hydrolytic activity (as normalized to the control) of compositions obtained from exemplary purification platforms. The hydrolytic activity rates were measured using a FAMS assay.

FIG. 6 shows a bar graph of the hydrolytic activity (as normalized to the control) of compositions obtained from exemplary purification platforms. The hydrolytic activity rates were measured using a FAMS assay.

FIG. 7 shows a bar graph of the hydrolytic activity (as normalized to the control) of compositions obtained from exemplary purification platforms. The hydrolytic activity rates were measured using a FAMS assay.

### DETAILED DESCRIPTION

The present application provides, in some aspects, methods for purifying a target from a sample comprising the target, the methods comprising subjecting the sample to a purification platform disclosed herein comprising one or more depth filtration steps and/or one or more hydrophobic interaction chromatography (HIC) steps and/or one or more multimodal hydrophobic interaction/ion exchange (MM-HIC/IEX) chromatography steps. In some embodiments, the purified target is for use in a pharmaceutical composition. In some embodiments, the target is a polypeptide, such as a recombinant polypeptide, e.g., an antibody moiety.

The present disclosure is based, at least in part, on unexpected findings demonstrating that purification platforms comprising one or more depth filtration steps and/or one or more HIC steps and/or one or more MM-HIC/IEX chromatography steps reduces the hydrolytic enzyme activity rate of a composition obtained therefrom as compared to a composition obtained from a purification platform not having the one or more depth filtration steps and/or the one or more HIC steps and/or MM-HIC/IEX chromatography steps. The unexpected findings demonstrated that the purification platforms described herein are especially useful for purifying a target produced by a host cell. As discussed herein, certain host cell proteins and impurities, including host cell hydrolytic enzymes, may have a propensity to co-purify with the target. Such host cell hydrolytic enzymes can degrade the target and/or additives added to the target useful for preparing and formulating compositions thereof. The demonstrated reduction in hydrolytic enzyme activity rate in the compositions obtained from purification platforms described herein ensure that additives included in the compositions of a target, such as surfactants, e.g., a polysorbate, are not degraded by host cell impurities thereby improving the stability of the target and composition shelf-life.

Thus, in some aspects, provided herein is a method of reducing a hydrolytic enzyme activity rate of a composition obtained from a purification platform, the method comprising subjecting a sample to the purification platform comprising: a capture step; one or more ion exchange (IEX) chromatography steps; and a depth filtration step, thereby reducing the hydrolytic enzyme activity rate of the composition as compared to purification of the sample without the depth filtration step.

In other aspects, provided herein is a method of reducing a hydrolytic enzyme activity rate of a composition obtained from a purification platform, the method comprising subjecting a sample to the purification platform comprising: a capture step; a multimodal hydrophobic interaction/ion exchange (MM-HIC/IEX) chromatography step; and a hydrophobic interaction chromatography (HIC) step, thereby reducing the hydrolytic enzyme activity rate of the composition as compared to purification of the sample without the HIC step and/or the MM-HIC/IEX chromatography step.

In other aspects, provided herein is a method of reducing a hydrolytic enzyme activity rate of a composition obtained from a purification platform, the method comprising subjecting a sample to the purification platform comprising: a capture step; one or more ion exchange (IEX) chromatography steps; and a hydrophobic interaction chromatography (HIC) step, thereby reducing the hydrolytic enzyme activity rate of the composition as compared to purification of the sample without the HIC step.

In other aspects, provided herein is a method of reducing a hydrolytic enzyme activity rate of a composition obtained from a purification platform, the method comprising subjecting a sample to the purification platform comprising: one or more ion exchange (IEX) chromatography steps; a hydrophobic interaction chromatography (HIC) step; and a depth filtration step, thereby reducing the hydrolytic enzyme activity rate of the composition as compared to purification of the sample without the HIC step or the depth filtration step.

In other aspects, provided herein is a method of reducing a hydrolytic enzyme activity rate of a composition obtained from a purification platform, the method comprising subjecting a sample to the purification platform comprising: a capture step; one or more ion exchange (IEX) chromatography steps; a multimodal hydrophobic interaction/ion exchange (MM-HIC/IEX) chromatography step; and one or both of: a hydrophobic interaction chromatography (HIC) step; and a depth filtration step, thereby reducing the hydrolytic enzyme activity rate of the composition as compared to purification of the sample without the HIC step or the depth filtration step and/or MM-HIC/IEX chromatography step.

In other aspects, provided herein is a pharmaceutical composition obtained from a method described herein.

In other aspects, provided herein is a formulated antibody moiety composition comprising an antibody moiety and a polysorbate, wherein the composition has a reduced rate of polysorbate hydrolysis, wherein the shelf-life of the composition is more than 12 months.

In other aspects, provided herein is a formulated antibody moiety composition comprising an antibody moiety and a polysorbate, wherein the composition has a reduced rate of polysorbate hydrolysis, wherein the shelf-life of the composition is extended compared to the shelf-life indicated in documents filed with a health authority related to the formulated antibody moiety composition, wherein the shelf-life is extended by at least 3 months compared to the shelf-life indicated in said documents.

In other aspects, provided herein is a formulated antibody moiety composition comprising an antibody moiety, wherein the formulated antibody moiety composition has a reduced degradation of polysorbate, wherein the degradation is reduced by at least about 50% compared to the degradation indicated in documents filed with a health authority related to the formulated antibody moiety composition.

In other aspects, provided herein is a formulated antibody moiety composition comprising an antibody moiety and a polysorbate, wherein the polysorbate is degraded during storage of the liquid composition by 50% or less per year.

It will also be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects.

### I. Definitions

For purposes of interpreting this specification, the following definitions will apply and, whenever appropriate, terms used in the singular will also include the plural and vice versa. In the event that any definition set forth below conflicts with any document incorporated herein by reference, the definition set forth shall control.

The term “antibody moiety” includes full-length antibodies and antigen-binding fragments thereof. In some embodiments, a full-length antibody comprises two heavy chains and two light chains. The variable regions of the light and heavy chains are responsible for antigen binding. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of Kabat, Chothia, or Al-Lazikani (Al-Lazikani 1997; Chothia 1985; Chothia 1987; Chothia 1989; Kabat 1987; Kabat 1991). The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding, but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, ε, γ, and μ heavy chains, respectively. Several of the major antibody classes are divided into subclasses such as lgG1 (71 heavy chain), lgG2 (γ2 heavy chain), lgG3 (γ3 heavy chain), lgG4 (γ4 heavy chain), lgA1 (α1 heavy chain), or lgA2 (α2 heavy chain). In some embodiments, the antibody moiety is a chimeric antibody. In some embodiments, the antibody moiety is a semi-synthetic antibody. In some embodiments, the antibody moiety is a diabody. In some embodiments, the antibody moiety is a humanized antibody. In some embodiments, the antibody moiety is a multispecific antibody, such as a bispecific antibody. In some embodiments, the antibody moiety is linked to a fusion protein. In some embodiments the antibody moiety is linked to an immunostimulating protein, such as an interleukin. In some embodiments the antibody moiety is linked to a protein which facilitates the entry across the blood brain barrier.

The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “chimeric antibodies” refer to antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this invention (see U.S. Pat. No. 4,816,567; and Morrison et al., Proc. Natl. Acad. Sci. USA, 81:6851-6855 (1984)).

The term “multispecific antibodies” as used herein refer to monoclonal antibodies that have binding specificities for at least two different sites, i.e., different epitopes on different antigens or different epitopes on the same antigen. In certain aspects, the multispecific antibody has two binding specificities (bispecific antibody). In certain aspects, the multispecific antibody has three or more binding specificities. Multispecific antibodies may be prepared as full length antibodies or antibody fragments.

The term “semi-synthetic” in reference to an antibody or antibody moiety means that the antibody or antibody moiety has one or more naturally occurring sequences and one or more non-naturally occurring (i.e., synthetic) sequences.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the heavy and light chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv,” also abbreviated as “sFv” or “scFv,” are antibody fragments that comprise the V_{H} and V_{L} antibody domains connected into a single polypeptide chain. In some embodiments, the scFv polypeptide further comprises a polypeptide linker between the V_{H} and V_{L} domains which enables the scFv to form the desired structure for antigen binding. For a review of scFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments prepared by constructing scFv fragments (see preceding paragraph) typically with short linkers (such as about 5 to about 10 residues) between the V_{H} and V_{L} domains such that inter-chain but not intra-chain pairing of the V domains is achieved, resulting in a bivalent fragment, i.e., fragment having two antigen-binding sites. Bispecific diabodies are heterodimers of two “crossover” scFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region (HVR) of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictates otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.

As used herein, including in the appended claims, the singular forms “a,” “or,” and “the” include plural referents unless the context clearly dictates otherwise.

### II. Purification Platforms and Steps Thereof

In some aspects, provided herein are purification platforms designed for purifying a target from a sample, wherein the purification platforms comprise one or more depth filtration steps and/or one or more HIC steps and/or one or more multimodal hydrophobic interaction chromatography/ion exchange (MM-HIC/IEX) chromatography steps. The described purification platforms are useful for obtaining a purified composition having a reduced hydrolytic enzyme activity rate as compared to purification of the sample without the depth filtration step and/or the HIC step and/or one or more MM-HIC/IEX chromatography steps. In some embodiments, the purification platform comprising a depth filtration step. In some embodiments, the purification platform comprises a HIC step. In some embodiments, the purification platform comprises a depth filtration step and a HIC step. In some embodiments, the purification platform further comprises one or more additional steps, wherein each step is selected from an ion exchange (IEX) chromatography step, a MM-HIC/IEX chromatography step, a capture step, a virus filtration step, an ultrafiltration/diafiltration (UF/DF) step, and a conditioning step.

In some embodiments, the purification platform represents a workflow for purifying, to any degree, a target from a sample comprising the target. In some instances of the present disclosure, descriptions of components and features of the purification platforms, and methods of use thereof, are provided in a modular manner. One of ordinary skill in the art will readily understand that such disclosure is not meant to limit the scope of the present application, and that the disclosure encompasses numerous arrangements of purification platforms, or features thereof, encompassed by the description herein. For example, in some embodiments, a specific type of chromatography medium may readily be understood to be encompassed by the description of purification platforms as comprising a genus of the chromatography medium. Certain features and embodiments of the purification platforms encompassed herein, such as methods for performing a step thereof, are described in PCT/US2020/031164, which is hereby incorporated herein by reference in its entirety.

Furthermore, one of ordinary skill in the art will recognize that certain chromatography medium described herein may have more than one different feature dictating the interaction of the medium with a component, such as a target. For example, a multimodal HIC/IEX chromatography medium may have a ligand having both a hydrophobic interaction feature and an electrostatic interaction feature. Description of a chromatography medium in a section below does not limit the types of features that may be present thereon.

### A. Depth Filtration Steps

In some embodiments, the purification platform described herein comprises a depth filtration step. As described herein, a depth filtration step can be placed at any of one or more positions within a purification platform. In some embodiments, the purification platform described herein comprises one or more depth filtration steps, such as any of 2, 3, 4, or 5 depth filtration steps, positioned at any stage of the process workflow. In some embodiments, wherein the purification platform comprises more than one depth filtration step, the depth filtration steps are not performed in direct sequential order, i.e., without some intervening step of the purification platform performed between the depth filtration steps. In some embodiments, wherein the purification platform comprises more than one depth filtration step, the depth filtration steps are the same. In some embodiments, wherein the purification platform comprises more than one depth filtration step, the depth filtration steps are different, e.g., comprise use of a different depth filter.

In some embodiments, the depth filtration step is used as a load filtration in conjugation with another aspect of the purification platform. In some embodiments, the use of the term “step,” in “depth filtration step,” does not exclude purification platforms wherein the depth filtration feature of the purification platform is directly combined with another feature, e.g., the depth filtration eluate flows directly to a subsequent feature or step of the purification platform.

Depth filtration steps, including what is involved with the processing via a depth filter, are known in the art. See, e.g., Yigzaw et al., Biotechnol Prog, 22, 2006, and Liu et al., mAbs, 2, 2010, which are hereby incorporated herein by reference in their entirety. Based on the state of the art and disclosure herein, one of ordinary skill in the art will understand components, conditions, and reagents involved with performing a depth filtration step.

In some embodiments, the depth filtration step comprises processing via a depth filter. In some embodiments, the depth filter comprises a porous filtration medium capable of retaining portions of a sample, such as cell components and debris, wherein filtration occurs, e.g., within the depth of the filter material. In some embodiments, the depth filter comprises synthetic material, non-synthetic material, or a combination thereof. In some embodiments, the depth filter comprises a substrate comprising one or more of a diatomaceous earth composition, a silica composition, a cellulose fiber, a polymeric fiber a polyacrylic fiber, a cohesive resin, a synthetic particulate, an ionic charged resin, and an ash composition. In some embodiments, the depth filter comprises diatomaceous earth. In some embodiments, the depth filter comprises anion exchange media. In some embodiments, the depth filter comprises a hydrophobic interaction medium. In some embodiments, at least a portion of the substrate of a depth filter comprises a surface modification. In some embodiments, the surface modification is one or more of a quaternary amine surface modification (such as a quaternary ammonium, Q, functionality), a guanidinium surface modification, a cationic surface modification, an anionic surface modification, and a hydrophobic modification. In some embodiments, the surface modification comprises a ligand having one or more features designed to facilitate an interaction with another component, such as the target; such features may include moieties involved with, e.g., hydrogen bonding, hydrophilic interactions, hydrophobic interactions, and ionic interactions (cation and anion).

In some embodiments, the depth filtration step is configured to be performed at a pre-determined pH or range thereof, e.g., the input material has a pre-determined pH or range thereof. In some embodiments, the depth filtration step is configured to be performed at, e.g., the input material has, a pH of about 4.5 to about 9.5, such as any of about 4.5 to about 7, about 5 to about 6, about 5 to about 5.5, about 7 to about 9.5, about 4.5 to about 9, about 5 to about 8.5, or about 7.5 to about 8.5. In some embodiments, the depth filtration step is configured to be performed at, e.g., the input material has, a pH of at least about 4.5, such as at least about any of 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, or 9.5. In some embodiments, the depth filtration step is configured to be performed at, e.g., the input material has, a pH of less than about 9.5, such as less than about any of 9.4, 9.3, 9.2, 9.1, 9, 8.9, 8.8, 8.7, 8.6, 8.5, 8.4, 8.3, 8.2, 8.1, 8, 7.9, 7.8, 7.7, 7.6, 7.5, 7.4, 7.3, 7.2, 7.1, 7, 6.9, 6.8, 6.7, 6.6, 6.5, 6.4, 6.3, 6.2, 6.1, 6, 5.9, 5.8, 5.7, 5.6, 5.5, 5.4, 5.3, 5.2, 5.1, 5, 4.9, 4.8, 4.7, 4.6, or 4.5. In some embodiments, the depth filtration step is configured to be performed at, e.g., the input material has, a pH of about any of 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, or 9.5.

In some embodiments, the depth filter is selected from the group consisting of: a X0SP depth filter, a D0SP depth filter, a C0SP depth filter, an EMPHAZE™ depth filter, a PDD1 depth filter, a PDE1 depth filter, a PDH5 depth filter, a ZETA PLUS™ 120ZA depth filter, a ZETA PLUS™ 120ZB depth filter, a ZETA PLUS™ DELI depth filter, and a ZETA PLUS™ DELP depth filter.

In some embodiments, the depth filter comprises a silica material, such as a silica filter aid, with or without a polyacrylic fiber. In some embodiments, the depth filter comprises two or more layers of filter media, wherein a first layer comprises a silica material, such as a silica filter aid, and a second layer comprises a polyacrylic fiber, such as a polyacrylic fiber pulp. In some embodiments, the depth filter is a depth filter comprising synthetic material and does not comprise diatomaceous earth and/or perlite. In some embodiments, the depth filter is a X0SP depth filter. In some embodiments, the depth filter is a D0SP depth filter. In some embodiments, the depth filter is a C0SP depth filter.

In some embodiments, the silica filter aid is a precipitated silica filter aid. In some embodiments, the filter aid is an aspect of the filter, such as a layer, that aids with performing the filter function. In some embodiments, the silica filter aid is a silica gel filter aid. In some embodiments, the silica filter aid has about 50% of silanols ionized at pH 7. In some embodiments, the silica filter aid is a silica gel filter aid, wherein about 50% of silanols of the silica filter aid are ionized at pH 7. In some embodiments, the silica filter aid is precipitated from silicas, such as SIPERNAT® (Evonik Industries AG), or silica gels, such as Kieseigel 60 (Merck KGaA). In some embodiments, the polyacrylic fiber is a non-woven polyacrylic fiber pulp. In some embodiments, the polyacrylic fiber is an electrospun polyacrylic nanofiber. In some embodiments, the degree of fibrillation of the polyacrylic fibers correlates with a Canadian Standard Freeness (CSF) from about 10 mL to about 800 mL. In some embodiments, the depth filter has a pore size of about 0.05 μm to about 0.2 μm, such as about 0.1 μm. In some embodiments, the depth filter has a surface area of about 0.1 m² to about 1.5 m², such as about 0.11 m², about 0.55 m², 0.77 m², or about 1.1 m². In some embodiments, the depth filter does not comprise diatomaceous earth and/or perlite. In some embodiments, the depth filter comprises two layers of filter media, wherein a first layer comprises a silica filter aid having about 50% of silanols ionized at pH 7, and a second layer comprises a polyacrylic fiber pulp having a degree of fibrillation of the polyacrylic fibers correlating with a Canadian Standard Freeness (CSF) from about 10 mL to about 800 mL, and wherein the depth filter does not comprise diatomaceous earth.

In some embodiments, the depth filter comprises a silica, such as a silica filter aid, and a polyacrylic fiber, such as a X0SP depth filter, a C0SP depth filter, or a D0SP depth filter, wherein the depth filtration step is configured to be performed at, e.g., the input material has, a pH of about 4.5 to about 8, about 5 to about 6, about 5.3 to about 5.7, or about 7.3 to about 7.7. In some embodiments, the depth filter comprises a silica, such as a silica filter aid, and a polyacrylic fiber, such as a X0SP depth filter, a C0SP depth filter, or a D0SP depth filter, wherein the depth filtration step is configured to be performed at, e.g., the input material has, a pH of at least about 4.5, such as at least about any of 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, or 8. In some embodiments, the depth filter comprises a silica, such as a silica filter aid, and a polyacrylic fiber, such as a X0SP depth filter, a C0SP depth filter, or a D0SP depth filter, wherein the depth filtration step is configured to be performed at, e.g., the input material has, a pH of less than about 8, such as less than about any of 7.9, 7.8, 7.7, 7.6, 7.5, 7.4, 7.3, 7.2, 7.1, 7, 6.9, 6.8, 6.7, 6.6, 6.5, 6.4, 6.3, 6.2, 6.1, 6, 5.9, 5.8, 5.7, 5.6, 5.5, 5.4, 5.3, 5.2, 5.1, 5, 4.9, 4.8, 4.7, 4.6, or 4.5. In some embodiments, the depth filter comprises a silica, such as a silica filter aid, and a polyacrylic fiber, such as a X0SP depth filter, a C0SP depth filter, or a D0SP depth filter, wherein the depth filtration step is configured to be performed at, e.g., the input material has, a pH of about any of 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, or 8.

In some embodiments, the depth filter comprises a hydrogel Q (quaternary amine, also referred to as quaternary ammonium)-functionalized non-woven material, and a multizone microporous membrane. In some embodiments, the depth filter comprises four layers comprising hydrogel Q-functionalized non-woven materials, and a nine-zone microporous membrane. In some embodiments, the non-woven material comprises polypropylene. In some embodiments, the depth filter is a depth filter comprising synthetic material and does not comprise diatomaceous earth and/or perlite. In some embodiments, the depth filter is an EMPHAZE™ depth filter, e.g., an EMPHAZE™ AEX depth filter.

In some embodiments, the depth filter comprises multiple components or layers. In some embodiments, the depth filter comprises multiple layers comprising one or more layers comprising anion-exchange (AEX) functional polymers. In some embodiments, the layer comprising AEX functional polymers comprises a quaternary ammonium (Q), such as a Q functional hydrogel. In some embodiments, the layer comprising AEX functional polymers comprises a quaternary ammonium (Q) functional polymer associated with a non-woven article. In some embodiments, the layer comprising AEX functional polymers comprises a quaternary ammonium (Q) functional hydrogel covalently grafted to a fine-fiber polypropylene non-woven scaffold. In some embodiments, the depth filter comprises multiple layers comprising a layer comprising a multi-zone membrane comprising a nine-zone membrane with a pore size of about 0.05 μm to about 0.3 μm, such as about 0.22 μm. In some embodiments, the depth filter does not comprise diatomaceous earth.

In some embodiments, the depth filter comprises a hydrogel Q (quaternary amine, also referred to as a quaternary ammonium)-functionalized non-woven material, such as an EMPHAZE™ AEX depth filter, wherein the depth filtration step is configured to be performed at, e.g., the input material has, a pH of about 7 to about 9.5, such as any of about 7.5 to about 8.5 or about 7.8 to about 8.2. In some embodiments, the depth filter comprises a hydrogel Q (quaternary amine, also referred to as a quaternary ammonium)-functionalized non-woven material, such as an EMPHAZE™ AEX depth filter, wherein the depth filtration step is configured to be performed at, e.g., the input material has, a pH of at least about 7, such as at least about any of 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, or 9.5. In some embodiments, the depth filter comprises a hydrogel Q (quaternary amine, also referred to as a quaternary ammonium)-functionalized non-woven material, such as an EMPHAZE™ AEX depth filter, wherein the depth filtration step is configured to be performed at, e.g., the input material has, a pH of less than about 9.5, such as less than about any of 9.4, 9.3, 9.2, 9.1, 9, 8.9, 8.8, 8.7, 8.6, 8.5, 8.4, 8.3, 8.2, 8.1, 8, 7.9, 7.8, 7.7, 7.6, 7.5, 7.4, 7.3, 7.2, 7.1, or 7. In some embodiments, the depth filter comprises a hydrogel Q (quaternary amine, also referred to as a quaternary ammonium)-functionalized non-woven material, such as an EMPHAZE™ AEX depth filter, wherein the depth filtration step is configured to be performed at, e.g., the input material has, a pH of about any of 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, or 9.5.

In some embodiments, the depth filter comprises a Q (quaternary amine, also referred to as quaternary ammonium)-functionalized non-woven material, and a Gu (guanidinium)-functionalized membrane. In some embodiments, the Q-functionalized non-woven material is configured in one or more layers, such as any of 1, 2, or 3. In some embodiments, the Gu-functionalized membrane is configured in one or more layers, such as any of 1, 2, 3, or 4. In some embodiments, the depth filter comprises three layers of a Q-functionalized non-woven material and four layers of a Gu-functionalized membrane. In some embodiments, the non-woven material comprises polypropylene. In some embodiments, the Gu-functionalized membrane is a polyamide membrane. In some embodiments, the depth filter is a Polisher ST depth filter. In some embodiments, the depth filter comprises a Q-functionalized non-woven material, and a Gu-functionalized membrane, such as a Polisher ST depth filter, wherein the depth filtration step using the depth filter is configured to be performed at, e.g., the input material has, a pH of about any of 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0.

In some embodiments, the depth filter comprises a Q (quaternary amine, also referred to as quaternary ammonium)-functionalized non-woven material, and a Gu (guanidinium)-functionalized membrane, such as a Polisher ST depth filter, wherein the depth filtration step is configured to be performed at, e.g., the input material has, a pH of about 4.5 to about 9, such as any of about 5 to about 8 or about 7 to about 9. In some embodiments, the depth filter comprises a Q (quaternary amine, also referred to as quaternary ammonium)-functionalized non-woven material, and a Gu (guanidinium)-functionalized membrane, such as a Polisher ST depth filter, wherein the depth filtration step is configured to be performed at, e.g., the input material has, a pH of at least about 4.5, such as at least about any of 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In some embodiments, the depth filter comprises a Q (quaternary amine, also referred to as quaternary ammonium)-functionalized non-woven material, and a Gu (guanidinium)-functionalized membrane, such as a Polisher ST depth filter, wherein the depth filtration step is configured to be performed at, e.g., the input material has, a pH of less than about 9.0, such as less than about any of 8.9, 8.8, 8.7, 8.6, 8.5, 8.4, 8.3, 8.2, 8.1, 8, 7.9, 7.8, 7.7, 7.6, 7.5, 7.4, 7.3, 7.2, 7.1, 7, 6.9, 6.8, 6.7, 6.6, 6.5, 6.4, 6.3, 6.2, 6.1, 6.0, 5.9, 5.8, 5.7, 5.6, 5.5, 5.4, 5.3, 5.2, 5.1, 5.0, 4.9, 4.8, 4.7, 4.6, or 4.5. In some embodiments, the depth filter comprises a Q (quaternary amine, also referred to as quaternary ammonium)-functionalized non-woven material, and a Gu (guanidinium)-functionalized membrane, such as a Polisher ST depth filter, wherein the depth filtration step is configured to be performed at, e.g., the input material has, a pH of about any of 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0.

In some embodiments, the depth filter comprises cellulose fibers, diatomaceous earth, and perlite. In some embodiments, the depth filter comprises two layers, wherein each layer comprises a cellulose filter matrix, and wherein the cellulose filter matrix is impregnated with a filter aid comprising one or more of diatomaceous earth or perlite. In some embodiments, the depth filter comprises two layers, wherein each layer comprises a cellulose filter matrix, wherein the cellulose filter matrix is impregnated with a filter aid comprising one or more of diatomaceous earth or perlite, and wherein each layer further comprises a resin binder. In some embodiments, the depth filter is a PDD1 depth filter. In some embodiments, the depth filter is a PDE1 depth filter. In some embodiments, the depth filter is a PDH5 depth filter.

### B. HIC Steps

In some embodiments, the purification platform described herein comprises a HIC step. As described herein, a HIC step can be placed at any one or more positions within a purification platform. In some embodiments, the purification platform described herein comprises one or more HIC steps, such as any of 2, 3, 4, or 5 HIC steps, positioned at any stage of the process workflow. In some embodiments, wherein the purification platform comprises more than one HIC steps, the HIC steps are not performed in direct sequential order, i.e., without some intervening step of the purification platform performed between the HIC steps. In some embodiments, wherein the purification platform comprises more than one HIC step, the HIC steps are the same. In some embodiments, wherein the purification platform comprises more than one HIC step, the HIC steps are different, e.g., comprise use of a different HIC medium.

In some embodiments, the HIC step is used in conjugation with another aspect of the purification platform. In some embodiments, the use of the term “step,” in “HIC step,” does not exclude purification platforms wherein the HIC feature of the purification platform is directly combined with another feature, e.g., an eluate flows directly to HIC feature. In some embodiments, the HIC step comprises a chromatography medium comprising a HIC feature, including a multimodal chromatography medium comprising a HIC feature such as MM-HIC/IEX.

In some embodiments, the HIC step comprises processing via a HIC medium, such as a HIC column and/or HIC membrane. HIC steps, including what is involved with the processing via a HIC medium, are known in the art. See, e.g., Liu et al. mAbs, 2, 2010, which is hereby incorporated herein by reference in its entirety. Based on the state of the art and disclosure herein, one of ordinary skill in the art will understand, for example, components, conditions, and reagents involved with a HIC step.

HIC steps allow for separation based on hydrophobic interactions between a hydrophobic ligand of the HIC medium and a component of a sample, e.g., a target or non-target component. For example, in some embodiments, a high salt condition is used to reduce the solvation of the target thereby exposing hydrophobic regions which can then interact with the HIC medium. In some embodiments, the HIC medium comprises a substrate, such as an inert matrix, e.g., a cross-linked agarose, sepharose, or resin matrix. In some embodiments, at least a portion of the substrate of a HIC medium comprises a surface modification comprising the hydrophobic ligand. In some embodiments, the HIC ligand is a ligand comprising between about 1 and 18 carbons. In some embodiments, the HIC ligand comprises 1 or more carbons, such as any of 2 or more carbons, 3 or more carbons, 4 or more carbons, 5 or more carbons, 6 or more carbons, 7 or more carbons, 8 or more carbons, 9 or more carbons, 10 or more carbons, 11 or more carbons, 12 or more carbons, 13 or more carbons, 14 or more carbons, 15 or more carbons, 16 or more carbons, 17 or more carbons, or 18 or more carbons. In some embodiments, the HIC ligand comprises any of 1 carbon, 2 carbons, 3 carbons, 4 carbons, 5 carbons, 6 carbons, 7 carbons, 8 carbons, 9 carbons, 10 carbons, 11 carbons, 12 carbons, 13 carbons, 14 carbons, 15 carbons, 16 carbons, 17 carbons, or 18 carbons. In some embodiments, the hydrophobic ligand is selected from the group consisting of an ether group, a methyl group, an ethyl group, a propyl group, an isopropyl group, a butyl group, a t-butyl group, a hexyl group, an octyl group, a phenyl group, and a polypropylene glycol group. In some embodiments, the HIC medium is a hydrophobic charge induction chromatography medium.

In some embodiments, the HIC step is configured to be performed at a pre-determined pH or range thereof, e.g., the input material has a pre-determined pH or range thereof. In some embodiments, the HIC step is configured to be performed at, e.g., the input material has, a pH of about 4.5 to about 7, such as any of about 5 to about 6, about 5 to about 5.5, or about 5.3 to about 5.7. In some embodiments, the HIC step is configured to be performed at, e.g., the input material has, a pH of at least about 4.5, such as at least about any of 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7. In some embodiments, the HIC step is configured to be performed at, e.g., the input material has, a pH of less than about 7, such as less than about any of 6.9, 6.8, 6.7, 6.6, 6.5, 6.4, 6.3, 6.2, 6.1, 6, 5.9, 5.8, 5.7, 5.6, 5.5, 5.4, 5.3, 5.2, 5.1, 5, 4.9, 4.8, 4.7, 4.6, or 4.5. In some embodiments, the HIC step is configured to be performed at, e.g., the input material has, a pH of about any of 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

In some embodiments, the HIC step comprises processing via a HIC medium, wherein processing via the HIC medium is performed in a bind-and-elute mode (i.e., the HIC step is a bind-and-elute mode HIC step). In some embodiments, the target polypeptide is eluted from a HIC medium using a step-wise elution with an aqueous buffer with decreasing salt concentrations, increasing concentrations of detergent, and/or an adjusted pH.

In some embodiments, the HIC step comprises processing via a HIC medium, wherein processing via the HIC medium is performed in a flow-through mode (i.e., the HIC step is a flow-through mode HIC step). In some embodiments, processing via the HIC membrane or the HIC column is performed using a low salt condition, e.g., no salt, such as a HIC conditioning salt, is added prior to loading a material on the HIC membrane or the HIC column. For example, in some embodiments, the HIC step does not comprise conductivity adjustment by the addition of a salt, such as a HIC condition salt. In some embodiments, the HIC condition salt comprises one or more of sodium sulfate, ammonium sulfate, sodium citrate, potassium phosphate, sodium phosphate, or any other salt used to condition a load for HIC. In some embodiments, the HIC step is a flow-through mode HIC step performed using an equilibration buffer and a wash buffer comprising sodium acetate at a pH of about 4.5 to about 6, such as about 5 or about 6.

In some embodiments, the HIC medium, such as the HIC membrane or the HIC column, is selected from the group consisting of Bakerbond WP HI-Propyl™, Phenyl Sepharose® Fast Flow (Phenyl-SFF), Phenyl Sepharose® Fast Flow Hi-sub (Phenyl-SFF HS), Toyopearl® Hexyl-650, Poros™ Benzyl Ultra, and Sartobind® phenyl. In some embodiments, the Toyopearl® Hexyl-650 is Toyopearl® Hexyl-650M. In some embodiments, the Toyopearl® Hexyl-650 is Toyopearl® Hexyl-650C. In some embodiments, the Toyopearl® Hexyl-650 is Toyopearl® Hexyl-650S.

In some embodiments, the HIC medium comprises propyl groups covalently linked to nitrogens on polyethylenimine (PEI) ligands attached to a substrate. In some embodiments, the HIC medium is Bakerbond WP HI-Propyl™. In some embodiments, the substrate is a particle, wherein the particle has an average size of 40 μm (e.g., Bakerbond WP HI-Propyl™ C3).

In some embodiments, the HIC medium comprises a cross-linked 6% agarose bead modified with aromatic phenyl groups via an uncharged and chemically-stable ether linkage. In some embodiments, the agarose beads have an average diameter of 90 μm. In some embodiments, the HIC medium is Phenyl Sepharose® Fast Flow (Phenyl SFF). As described herein, generic reference to Phenyl SFF includes both Sepharose® Fast Flow Low Sub (Phenyl SFF LS) and Sepharose® Fast Flow High Sub (Phenyl SFF HS), unless otherwise specified. In some embodiments, the HIC medium comprises about 15 μmol phenyl/mL to about 30 μmol phenyl/mL medium, including approximately 20 μmol phenyl/mL medium or approximately 25 μmol phenyl/mL medium. In some embodiments, the HIC medium is Phenyl Sepharose® Fast Flow Low Sub (Phenyl SFF LS). In some embodiments, the HIC medium comprises about 35 μmol phenyl/mL to about 50 μmol phenyl/mL medium, including approximately 40 μmol phenyl/mL medium or approximately 45 μmol phenyl/mL medium. In some embodiments, the HIC medium is Phenyl Sepharose® Fast Flow High Sub (Phenyl SFF HS).

In some embodiments, the HIC medium comprises 100 nm pore size polymethacrylate-based material bonded with C6 groups (hexyl). In some embodiments, the HIC medium comprises polymethacrylate-based particles having a mean size of 100 μm and a mean pore size of 100 nm (e.g. Toyopearl® Hexyl-650C). In some embodiments, the HIC medium comprises polymethacrylate-based particles having a mean size of 65 μm and a mean pore size of 100 nm (e.g, Toyopearl® Hexyl-650M). In some embodiments, the HIC medium comprises polymethacrylate-based particles having a mean size of 35 μm and a mean pore size of 100 nm (e.g., Toyopearl® Hexyl-650S).

In some embodiments, the HIC medium comprises cross-linked poly(styrene-divinylbenzene) POROS™-based bead with aromatic hydrophobic benzyl ligands. In some embodiments, the HIC medium is Poros™ Benzyl Ultra.

In some embodiments, the HIC medium is a membrane adsorber comprising a hydrophobic ligand. In some embodiments, the HIC medium comprises a phenyl moiety conjugated to a stabilized reinforced cellulose filter. In some embodiments, the HIC medium is Sartobind® Phenyl. In some embodiments, the HIC medium comprises a phenyl moiety conjugated to a stabilized reinforced cellular filter, e.g., Sartobind® Phenyl, wherein the HIC step is configured to be performed at, e.g., the input material has, a pH of about 4.5 to about 7, about 5 to about 6, or about 5.3 to about 5.7. In some embodiments, the HIC medium comprises a phenyl moiety conjugated to a stabilized reinforced cellular filter, e.g., Sartobind® Phenyl, wherein the HIC step is configured to be performed at, e.g., the input material has, a pH of at least about 4.5, such as at least about any of 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7. In some embodiments, the HIC medium comprises a phenyl moiety conjugated to a stabilized reinforced cellular filter, e.g., Sartobind® Phenyl, wherein the HIC step is configured to be performed at, e.g., the input material has, a pH of less than about 7, such as less than about any of 6.9, 6.8, 6.7, 6.6, 6.5, 6.4, 6.3, 6.2, 6.1, 6, 5.9, 5.8, 5.7, 5.6, 5.5, 5.4, 5.3, 5.2, 5.1, 5, 4.9, 4.8, 4.7, 4.6, or 4.5. In some embodiments, the HIC medium comprises a phenyl moiety conjugated to a stabilized reinforced cellular filter, e.g., Sartobind® Phenyl, wherein the HIC step is configured to be performed at, e.g., the input material has, a pH of about any of 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7.

### C. Ion Exchange Chromatography Steps

In some embodiments, the purification platform described herein comprise one or more ion exchange (IEX) chromatography steps. As described herein, an IEX chromatography step can be placed at any one or more positions within a purification platform. In some embodiments, the one or more IEX chromatography steps comprise more than one, such as any of 2, 3, 4, or 5, IEX chromatography steps, position at any stage of the process workflow. In some embodiments, wherein the purification platform comprises more than one IEX chromatography steps, the IEX chromatography steps are not performed in direct sequential order, i.e., without some intervening step of the purification platform performed between the IEX chromatography steps. In some embodiments, wherein the purification platform comprises more than one IEX chromatography step, the IEX chromatography steps are the same. In some embodiments, wherein the purification platform comprises more than one IEX chromatography step, the IEX chromatography steps are different, e.g., comprise use of a different IEX chromatography medium.

In some embodiments, the IEX chromatography step is used in conjugation with another aspect of the purification platform. In some embodiments, the use of the term “step,” in “IEX chromatography step,” or term encompassed thereby, does not exclude purification platforms wherein the IEX chromatography feature of the purification platform is directly combined with another feature, e.g., an eluate flows directly to an IEX chromatography feature.

In some embodiments, the IEX chromatography step comprises processing via an IEX chromatography medium, such as an IEX column and/or IEX membrane. IEX chromatography steps, including what is involved with processing for a polypeptide IEX chromatography step, are known in the art. See, e.g., Liu et al. mAbs, 2, 2010, which is hereby incorporated herein by reference in its entirety. Based on the state of the art and disclosure herein, one of ordinary skill in the art will understand, for example, components, conditions, and reagents involved with an IEX chromatography step.

In some embodiments, the IEX chromatography step is performed in a bind-and-elute mode (i.e., the IEX chromatography step is a bind-and-elute mode IEX chromatography step). In some embodiments, the IEX chromatography step is performed in a flow-through mode (i.e., the IEX chromatography step is a flow-through mode IEX chromatography step). In some embodiments, the IEX chromatography step is performed in an overload polypeptide purification step (i.e., the IEX chromatography step is an overload mode IEX chromatography step).

IEX chromatography steps allow for separation based on electrostatic interactions (anion and cation) between a ligand of the IEX chromatography medium and a component of a sample, e.g., a target or non-target component. In some embodiments, the IEX chromatography medium comprises a cation exchange (CEX) medium and/or feature. In some embodiments, the IEX chromatography medium comprises a strong CEX medium and/or feature. In some embodiments, the IEX chromatography medium comprises a weak CEX medium and/or feature. In some embodiments, the IEX chromatography medium comprises an anion exchange (AEX) medium and/or feature. In some embodiments, the IEX chromatography medium comprises a strong AEX medium and/or feature. In some embodiments, the IEX chromatography medium comprises a weak AEX medium and/or feature.

In some embodiments, the IEX chromatography medium is a multimodal ion exchange (MMIEX) chromatography medium. MMIEX chromatography media comprise both cation exchange and anion exchange components and/or features. In some embodiments, the MMIEX medium is a multimodal anion/cation exchange (MM-AEX/CEX) chromatography medium. In some embodiments, the IEX chromatography medium is a ceramic hydroxyapatite chromatography medium.

In some embodiments, the IEX chromatography medium, such as the IEX chromatography column medium or IEX chromatography membrane, is selected from the group consisting of: sulphopropyl (SP) Sepharose® Fast Flow (SPSFF), quartenary ammonium (Q) Sepharose® Fast Flow (QSFF), SP Sepharose® XL (SPXL), Streamline™ SPXL, ABx™ (MM-AEX/CEX medium), Poros™ XS, Poros™ 50HS, diethylaminoethyl (DEAE), dimethylaminoethyl (DMAE), trimethylaminoethyl (TMAE), quaternary aminoethyl (QAE), mercaptoethylpyridine (MEP)-Hypercel™, HiPrep™ Q XL, Q Sepharose® XL, and HiPrep™ SP XL.

In some embodiments, the AEX chromatography medium comprises a cross-linked 6% agarose bead having quaternary ammonium (Q) strong anion exchange groups. In some embodiments, the AEX chromatography medium is a strong anion exchanger medium. In some embodiments, the AEX chromatography medium is Q Sepharose® Fast Flow (QSFF).

In some embodiments, the CEX chromatography medium comprises cross-linked 6% agarose beads having sulphopropyl (SP) strong cation exchange groups. In some embodiments, the CEX chromatography medium is a strong cation exchanger medium. In some embodiments, the CEX chromatography medium is SP Sepharose® Fase Flow (SPSFF).

In some embodiments, the CEX chromatography medium comprises cross-linked 6% agarose beads having dextran chains covalently coupled to the agarose matrix that are modified with sulphopropyl (SP) strong cation exchange groups. In some embodiments, the CEX chromatography medium is a strong cation exchanger medium. In some embodiments, the CEX chromatography medium is SP Sepharose® XL (SPXL). In some embodiments, the CEX chromatography medium is Streamline™ SPXL.

In some embodiments, the CEX chromatography medium comprises rigid polymeric resin particles comprising cross-linked poly[styrene-divinylbenzene] having a polyhydroxyl surface coating further functionalization with sulphopropyl (SP) strong cation exchange groups. In some embodiments, the mean particle size is 50 μm. In some embodiments, the CEX chromatography medium is a strong cation exchanger medium. In some embodiments, the CEX chromatography medium is Poros™ XS.

In some embodiments, the CEX chromatography medium comprises rigid polymeric resin particles comprising cross-linked poly[styrene-divinylbenzene] having a polyhydroxyl surface coating further functionalization with sulphopropyl (SP) strong cation exchange groups. In some embodiments, the mean particle size is 50 μm. In some embodiments, the CEX chromatography medium is a strong cation exchanger medium. In some embodiments, the CEX chromatography medium is Poros™ 50HS.

In some embodiments, the MM-AEX/CEX chromatography medium comprises silica gel solid phase particles comprising a mixed mode anion/cation exchanger. In some embodiments, the silica gel solid phase particles have an average particle size of about 45 μm to about 65 μm. In some embodiments, the MM-AEX/CEX chromatography medium is Bakerbond ABx™.

### D. Multimodal Hydrophobic Interaction/Ion Exchange (MM-HIC/IEX) Chromatography Steps

In some embodiments, the purification platform described herein comprises a multimodal hydrophobic interaction/ion exchange (MM-HIC/IEX) chromatography step.

As described herein, a MM-HIC/IEX chromatography step can be placed at any one or more positions within a purification platform. In some embodiments, the purification platform described herein comprises one or more MM-HIC/IEX chromatography steps, such as any of 2, 3, 4, or 5 MM-HIC/IEX chromatography steps, positioned at any stage of the process workflow. In some embodiments, wherein the purification platform comprises more than one MM-HIC/IEX chromatography steps, the MM-HIC/IEX chromatography steps are not performed in direct sequential order, i.e., without some intervening step of the purification platform performed between the MM-HIC/IEX chromatography steps. In some embodiments, wherein the purification platform comprises more than one MM-HIC/IEX chromatography steps, two or more of the MM-HIC/IEX chromatography steps are the same. In some embodiments, wherein the purification platform comprises more than one MM-HIC/IEX chromatography steps, two or more of the MM-HIC/IEX chromatography steps are different, e.g., comprise use of a different MM-HIC/IEX chromatography medium.

In some embodiments, the MM-HIC/IEX chromatography step is used in conjugation with another aspect of the purification platform. In some embodiments, the use of the term “step,” in “MM-HIC/IEX chromatography step,” does not exclude purification platforms wherein the MM-HIC/IEX chromatography feature of the purification platform is directly combined with another feature, e.g., a depth filter is used a load filter in conjugation with a MM-HIC/IEX chromatography step.

In some embodiments, the MM-HIC/IEX chromatography medium comprises an anion exchange (AEX) material and/or feature (e.g., an MM-HIC/AEX chromatography medium). In some embodiments, the AEX material and/or feature is a strong anion exchange material and/or feature. In some embodiments, the AEX material and/or feature is a weak anion exchange material and/or feature.

In some embodiments, the MM-HIC/IEX chromatography medium comprises a cation exchange material and/or feature (e.g., an MM-HIC/CEX chromatography medium). In some embodiments, the CEX material and/or feature is a strong anion exchange material and/or feature. In some embodiments, the CEX material and/or feature is a weak anion exchange material and/or feature.

In some embodiments, the MM-HIC/IEX chromatography step comprises processing via a MM-HIC/IEX chromatography column or a MM-HIC/IEX chromatography membrane. In some embodiments, the MM-HIC/IEX chromatography column or membrane comprises an inert medium comprising a MM-HIC/IEX ligand. In some embodiments, the inert medium is a rigid agarose-based matrix, such as an agarose particle.

In some embodiments, the MM-HIC/IEX chromatography step comprises processing via Capto™ Adhere, Capto™ Adhere ImpRes, Capto™ MMC or Capto™ MMC ImpRes.

In some embodiments, the MM-HIC/AEX chromatography medium is a multimodal strong anion exchanger using a N-benzyl-N-methyl ethanolamine ligand. In some embodiments, the N-benzyl-N-methyl ethanolamine ligand is capable of protein interaction including hydrogen bonding, hydrophobic interactions, and electrostatic interactions (with anions). In some embodiments, the MM-HIC/AEX chromatography medium is Capto™ Adhere. In some embodiments, the MM-HIC/AEX chromatography medium is Capto™ Adhere ImpRes.

In some embodiments, the MM-HIC/CEX chromatography medium is a multimodal weak cation exchanger using a N-benzoyl-homocysteine ligand. In some embodiments, the N-benzoyl-homocysteine ligand is capable of protein interaction including hydrophobic interaction, hydrogen bonding, thiophilic interaction, and electrostatic interactions (with cations). In some embodiments, the MM-HIC/CEX chromatography medium is Capto™ MMC. In some embodiments, the MM-HIC/CEX chromatography medium is Capto™ MMC ImpRes.

### E. Capture Steps

In some embodiments, the purification platform comprises a capture step. In some embodiments, the capture steps described herein comprise use of an affinity chromatography, wherein the affinity chromatography utilizes an immobilized ligand to specifically bind to a target or a portion thereof, such as an antibody.

Capture steps, including what is involved with processing via, e.g., affinity chromatography, are known in the art. See, e.g., Liu et al. mAbs, 2, 2010, which is hereby incorporated by reference. Based on the state of the art and disclosure herein, one of ordinary skill in the art will understand components, conditions, and reagents involved with performing a capture step.

In some embodiments, the affinity chromatography is selected from the group consisting of a protein A chromatography, a protein G chromatography, a protein A/G chromatography, a FcXL chromatography, a protein XL chromatography, a kappa chromatography, and a kappaXL chromatography. In some embodiments, the affinity chromatography comprises use of an affinity medium, such as an inert matrix having an affinity ligand immobilized thereon, such as a protein A, or a portion thereof, immobilized thereon. In some embodiments, the affinity ligand is a protein A, or a portion thereof. In some embodiments, the protein A is a recombinant protein A. In some embodiments, the protein A is a native protein A. In some embodiments, the protein A is a protein A derivative, such as a polypeptide designed based on the protein A sequence and having certain modification, such as amino acid additions, deletions, and/or substitutions. In some embodiments, the inert matrix is a silica-based inert matrix, such as a silica-based filter or particle. In some embodiments, the inert matrix is an agarose-based matrix, such as an agarose particle. In some embodiments, the inert matrix is an organic polymer-based matrix, such as an organic polymer particle.

In some embodiments, the capture step comprises processing via an affinity chromatography. In some embodiments, the capture step comprises processing via a protein A chromatography. In some embodiments, the capture step is performed in a bind-and-elute mode.

In some embodiments, the protein A chromatography medium is selected from the group consisting of MabSelect™, MabSelect SuRe™, MabSelect SuRe™ LX, MabSelect Xtra™ MabSelect™ PrismA, ProSep®-vA, Prosep®-vA Ultra, Protein A Sepharose® Fast Flow, Poros® A, and MabCapture™.

In some embodiments, the protein A chromatography medium comprises a rigid, high-flow agarose matrix and alkali-stabilized protein A-derived ligand, wherein amino acids particularly sensitive to alkali were substituted with more stable residue in an alkali environment. In some embodiments, the matrix is a spherical particle. In some embodiments, the protein A chromatography medium is MabSelect SuRe™.

In some embodiments, the protein A chromatography medium comprises a rigid, high-flow agarose matrix and a protein A-derived ligand having alkaline stability. In some embodiments, the matrix is a spherical particle. In some embodiments, the protein A chromatography medium is MabSelect™ PrismA.

### F. Virus Filtration Steps

In some embodiments, the purification platform comprises a virus filtration step. In some embodiments, the virus filtration step is performed after one or more purification steps.

Virus filtration steps, including what is involved with processing for a virus filtration step, are known in the art. See, e.g., Liu et al. mAbs, 2, 2010, and U.S. Application No. 20140309403, which are hereby incorporated by reference. Based on the state of the art and disclosure herein, one of ordinary skill in the art will understand components, conditions, and reagents involved with performing a capture step.

In some embodiments, the virus filtration step comprises processing via a virus filter. In some embodiments, the virus filter comprises a pore size that retains both enveloped and non-enveloped viruses. In some embodiments, the pore size of the virus filter is based on the size of a target virus.

### G. Ultrafiltration/Diafiltration (UF/DF) Steps

In some embodiments, the purification platform comprises an UF/DF step. In some embodiments, the UF/DF step is performed after a step of the purification platform, such as an IEX chromatography step, a MM-HIC/IEX chromatography, a HIC step, a depth filtration step. In some embodiments, the UF/DF step is performed after a virus filtration step.

UF/DF steps, including what is involved with processing for an UF/DF step, are known in the art. See, e.g., Liu et al. mAbs, 2, 2010, which is hereby incorporated by reference. Based on the state of the art and disclosure herein, one of ordinary skill in the art will understand components, conditions, and reagents involved with performing a UF/DF step. In some embodiments, the UF/DF step comprises processing via ultrafiltration. In some embodiments, the UF/DF step is performed in tangential flow filtration (TFF) mode. In some embodiments, the UF/DF step comprises processing via a tangential flow filtration, such as high performance tangential flow filtration.

### H. Conditioning Steps

In some embodiments, the purification platform comprises a conditioning step. In some embodiments, the condition step comprises adjusting a feature or characteristic of a sample or a composition obtained from a purification platform to prior to subjecting the sample or the composition obtained from the purification platform to further processing. For example, in some embodiments, the condition step comprises adjusting a pH. In some embodiments, the condition step comprises adjusting a temperature. In some embodiments, the condition step comprises adjusting a buffer or salt concentration.

In some embodiments, the conditioning step is performed after a capture step. In some embodiments, the conditioning step is performed prior to a depth filtration step. In some embodiments, the conditioning step is performed prior to a HIC step. In some embodiments, the conditioning step is performed prior to a virus filtration step.

Conditioning steps, including what is involved with processing for a conditioning step, are known in the art. See, e.g., Liu et al. mAbs, 2, 2010, which is hereby incorporated by reference. Based on the state of the art and disclosure herein, one of ordinary skill in the art will understand components, conditions, and reagents involved with performing a conditioning step.

### I. Exemplary Purification Platforms

In some aspects provided herein is a purification platform for purifying a target from a sample, wherein the purification platforms comprise one or more depth filtration steps and/or one or more HIC steps and/or one or more MM-HIC/IEX chromatography steps, and wherein the purification platform is capable of obtaining a composition having a reduced hydrolytic enzyme activity rate as compared to a purification platform without the one or more depth filtration steps and/or the one or more HIC steps and/or the one or more MM-HIC/IEX chromatography steps. In some aspects, the reduction in the hydrolytic enzyme activity rate is at least about 20%, such as at least about any of 25%, 30%, 35%, 40%, 45%, 50%, 55%, 60%, 65%, 70%, 85%, 90%, or 95%.

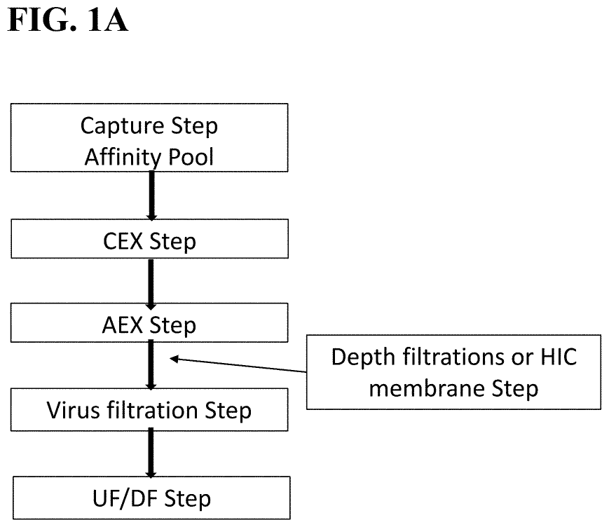

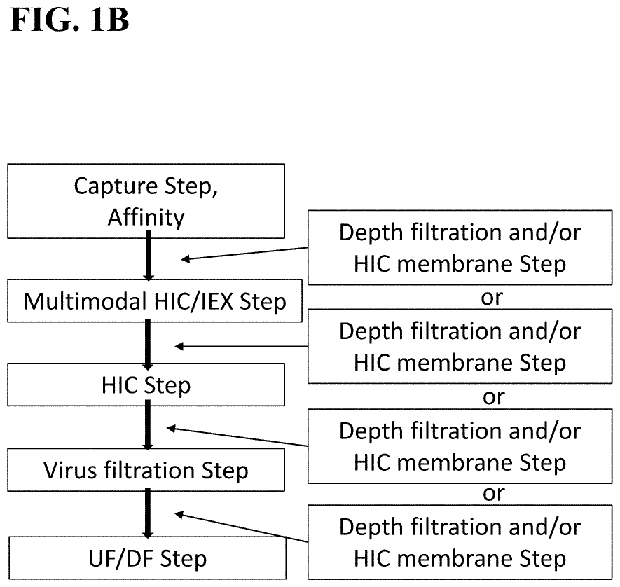

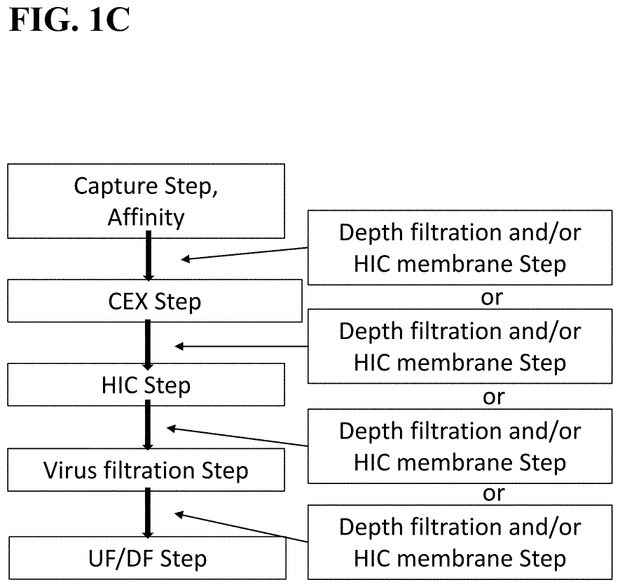

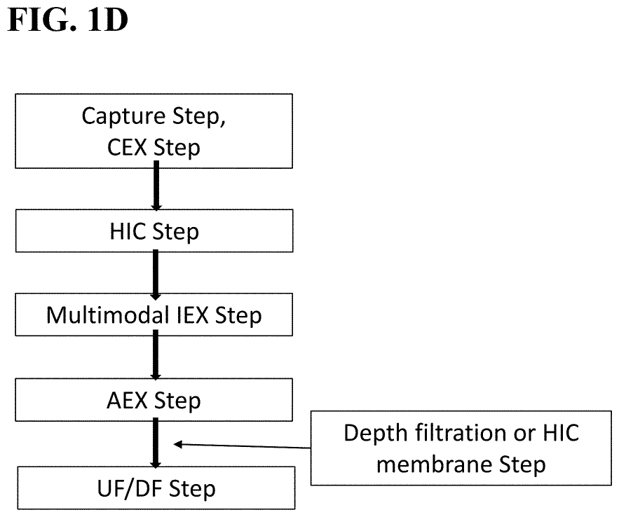

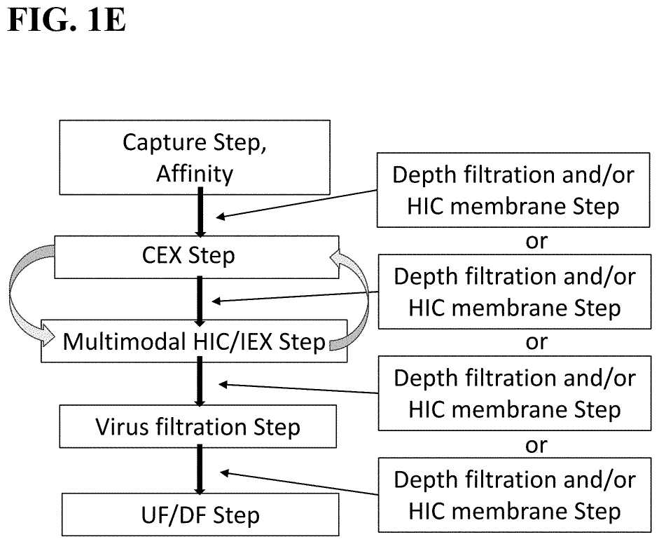

In some aspects, provided is a purification platform comprising: a capture step; one or more ion exchange (IEX) chromatography steps; and a depth filtration step. In some embodiments, the purification platform further comprises a virus filtration step and/or a UF/DF step. In some embodiments, the depth filtration step is directly prior to the virus filtration step or the UF/DF step. In some embodiments, the depth filtration step is sequential with or directly after an IEX chromatography step. In some embodiments, the depth filtration step comprising a X0SP depth filter on an EMPHAZE™ depth filter. In some embodiments, the purification platform further comprises a HIC step. Exemplary purification platforms encompassed within the described purification platforms is shown in FIG. 1A and described in more detail below.

In some embodiments, the purification platform comprises, in order: (a) a capture step; (b) a CEX chromatography step; (c) an AEX chromatography step; (d) a depth filtration step; (e) a virus filtration step; and (f) a UF/DF step. In some embodiments, the capture step comprises processing via a protein A-based affinity medium, e.g., a MabSelect SuRe™ or MabSelect™ PrimaA medium. In some embodiments, the capture step comprises a bind-and-elute mode affinity chromatography step. In some embodiments, the CEX chromatography step comprises a CEX chromatography medium comprises cross-linked 6% agarose beads having sulphopropyl (SP) strong cation exchange groups, e.g., a SP Sepharose® Fase Flow (SPSFF) medium. In some embodiments, the AEX chromatography step comprises an AEX chromatography medium comprising a cross-linked 6% agarose bead having quaternary ammonium (Q) strong anion exchange groups, e.g., a Q Sepharose® Fast Flow (QSFF) medium. In some embodiments, the depth filtration step is comprises a depth filter comprising a silica, such as a silica filter aid, and/or a polyacrylic fiber, e.g., a X0SP depth filter, a C0SP depth filter, or a D0SP depth filter.

In some embodiments, the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a CEX chromatography step, wherein the CEX chromatography step comprises a CEX chromatography medium comprises cross-linked 6% agarose beads having sulphopropyl (SP) strong cation exchange groups, e.g., a SP Sepharose® Fase Flow (SPSFF) medium; (c) an AEX chromatography step, wherein the AEX chromatography step comprises an AEX chromatography medium comprising a cross-linked 6% agarose bead having quaternary ammonium (Q) strong anion exchange groups, e.g., a Q Sepharose® Fast Flow (QSFF) medium; (d) a depth filtration step, wherein the depth filtration step comprises a depth filter comprising a silica, such as a silica filter aid, and/or a polyacrylic fiber, e.g., a X0SP depth filter, a C0SP depth filter, or a D0SP depth filter; a virus filtration step; and a UF/DF step. In some embodiments, the purification platform further comprises a depth filtration step, positioned at any position, wherein the depth filtration step comprises a depth filter comprising a silica, such as a silica filter aid, and/or a polyacrylic fiber, e.g., a X0SP depth filter, a C0SP depth filter, or a D0SP depth filter, and wherein the depth filtration step is configured to be performed at, e.g., the input material has, a pH of about 5 to about 6.5, such as about 5 to about 5.5 or about 5.8 to about 6.2. In some embodiments, the purification platform further comprises a depth filtration step, positioned at any position, wherein the depth filtration step comprises a depth filter comprising a hydrogel Q (quaternary amine, also referred to as a quaternary ammonium)-functionalized non-woven material, and a multizone microporous membrane, e.g., an EMPHAZE™ AEX depth filter, and wherein the depth filtration step is configured to be performed at, e.g., the input material has, a pH of about 7.5 to about 8.5, such as about 8. In some embodiments, the purification platform further comprises a HIC step, positioned at any position, wherein the HIC step comprises a HIC medium, such as a HIC membrane, comprising a phenyl moiety conjugated to a stabilized reinforced cellulose filter, e.g., a Sartobind® Phenyl medium, wherein the HIC step is configured to be performed at, e.g., the input material has, a pH of about 5 to about 6, such as about 5.5.

In some embodiments, the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a CEX chromatography step, wherein the CEX chromatography step comprises a CEX chromatography medium comprises cross-linked 6% agarose beads having sulphopropyl (SP) strong cation exchange groups, e.g., a SP Sepharose® Fase Flow (SPSFF) medium; (c) an AEX chromatography step, wherein the AEX chromatography step comprises an AEX chromatography medium comprising a cross-linked 6% agarose bead having quaternary ammonium (Q) strong anion exchange groups, e.g., a Q Sepharose® Fast Flow (QSFF) medium; (d) a depth filtration step, wherein the depth filtration step comprises a depth filter comprising a hydrogel Q (quaternary amine, also referred to as a quaternary ammonium)-functionalized non-woven material, and a multizone microporous membrane, e.g., an EMPHAZE™ AEX depth filter; a virus filtration step; and a UF/DF step. In some embodiments, the purification platform further comprises a depth filtration step, positioned at any position, wherein the depth filtration step comprises a depth filter comprising a silica, such as a silica filter aid, and a polyacrylic fiber, e.g., a X0SP depth filter, a C0SP depth filter, or a D0SP depth filter, and wherein the depth filtration step is configured to be performed at, e.g., the input material has, a pH of about 5 to about 6, such as about 5.5. In some embodiments, the purification platform further comprises a depth filtration step, positioned at any position, wherein the depth filtration step comprises a depth filter comprising a hydrogel Q (quaternary amine, also referred to as a quaternary ammonium)-functionalized non-woven material, and a multizone microporous membrane, e.g., an EMPHAZE™ AEX depth filter, and wherein the depth filtration step is configured to be performed at, e.g., the input material has, a pH of about 7.5 to about 8.5, such as about 8. In some embodiments, the purification platform further comprises a HIC step, positioned at any position, wherein the HIC step comprises a HIC medium, such as a HIC membrane, comprising a phenyl moiety conjugated to a stabilized reinforced cellulose filter, e.g., a Sartobind® Phenyl medium, wherein the HIC step is configured to be performed at, e.g., the input material has, a pH of about 5 to about 6, such as about 5.5.

In some embodiments, the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a CEX chromatography step, wherein the CEX chromatography step comprises a CEX chromatography medium comprises cross-linked 6% agarose beads having sulphopropyl (SP) strong cation exchange groups, e.g., a SP Sepharose® Fase Flow (SPSFF) medium; (c) an AEX chromatography step, wherein the AEX chromatography step comprises an AEX chromatography medium comprising a cross-linked 6% agarose bead having quaternary ammonium (Q) strong anion exchange groups, e.g., a Q Sepharose® Fast Flow (QSFF) medium; (d) a HIC step, wherein the HIC step comprises a HIC medium comprising a phenyl moiety conjugated to a stabilized reinforced cellulose filter, e.g., Sartobind® Phenyl; a virus filtration step; and a UF/DF step. In some embodiments, the purification platform further comprises a depth filtration step, positioned at any position, wherein the depth filtration step comprises a depth filter comprising a silica, such as a silica filter aid, and a polyacrylic fiber, e.g., a X0SP depth filter, a C0SP depth filter, or a D0SP depth filter, and wherein the depth filtration step is configured to be performed at, e.g., the input material has, a pH of about 5 to about 6, such as about 5.5. In some embodiments, the purification platform further comprises a depth filtration step, positioned at any position, wherein the depth filtration step comprises a depth filter comprising a hydrogel Q (quaternary amine, also referred to as a quaternary ammonium)-functionalized non-woven material, and a multizone microporous membrane, e.g., an EMPHAZE™ AEX depth filter, and wherein the depth filtration step is configured to be performed at, e.g., the input material has, a pH of about 7.5 to about 8.5, such as about 8. In some embodiments, the purification platform further comprises a HIC step, positioned at any position, wherein the HIC step comprises a HIC medium, such as a HIC membrane, comprising a phenyl moiety conjugated to a stabilized reinforced cellulose filter, e.g., a Sartobind® Phenyl medium, wherein the HIC step is configured to be performed at, e.g., the input material has, a pH of about 5 to about 6, such as about 5.5.

In some aspects, provided is a purification platform comprising: capture step; a multimodal hydrophobic interaction/ion exchange (MM-HIC/IEX) chromatography steps; and a hydrophobic interaction chromatography (HIC) step. In some embodiments, the purification platform further comprises a virus filtration step. In some embodiments, the purification platform further comprises a UF/DF step. Exemplary purification platforms encompassed within the described purification platforms is shown in FIG. 1B and described in more detail below.

In some embodiments, the purification platform comprises, in order: (a) a capture step; (b) a MM-HIC/IEX chromatography step; and (c) a HIC step. In some embodiments, the capture step comprises a protein A-based affinity medium, e.g., a MabSelect SuRe™ or MabSelect™ PrimaA medium. In some embodiments, the capture step comprises a bind-and-elute mode affinity chromatography step. In some embodiments, the MM-HIC/IEX chromatography step comprises a MM-HIC/AEX chromatography step. In some embodiments, the MM-HIC/AEX chromatography step comprises a medium comprising a multimodal strong anion exchanger using a N-benzyl-N-methyl ethanolamine ligand, e.g., a Capto™ Adhere medium. In some embodiments, the MM-HIC/IEX chromatography step is a MM-HIC/CEX chromatography step. In some embodiments, the MM-HIC/CEX chromatography step comprises a medium comprising a multimodal weak cation exchanger using a N-benzoyl-homocysteine ligand, e.g., a Capto™ MMC medium. In some embodiments, the HIC step comprises a medium comprising a cross-linked 6% agarose bead modified with aromatic phenyl groups via an uncharged and chemically-stable ether linkage, e.g., a Phenyl SFF medium, such as Phenyl SFF HS or Phenyl SFF LS. In some embodiments, the HIC step comprises a medium comprising a 100 nm pore size polymethacrylate-based material bonded with C6 groups (hexyl), e.g., a Toyopearl® Hexyl-650 medium, such as Toyopearl® Hexyl-650C. In some embodiments, the HIC step comprises a medium comprising a cross-linked poly(styrene-divinylbenzene) POROS™-based bead with aromatic hydrophobic benzyl ligands, e.g., Poros™ Benzyl Ultra.

In some embodiments, provided is a purification platform comprising, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/AEX chromatography step, wherein the MM-HIC/AEX chromatography step comprises a medium comprising a multimodal strong anion exchanger using a N-benzyl-N-methyl ethanolamine ligand, e.g., a Capto™ Adhere medium; and (c) a HIC step, wherein the HIC step comprises a medium comprising a cross-linked 6% agarose bead modified with aromatic phenyl groups via an uncharged and chemically-stable ether linkage, e.g., a Phenyl SFF medium, such as Phenyl SFF HS or Phenyl SFF LS.

In some embodiments, provided is a purification platform comprising, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/AEX chromatography step, wherein the MM-HIC/AEX chromatography step comprises a medium comprising a multimodal strong anion exchanger using a N-benzyl-N-methyl ethanolamine ligand, e.g., a Capto™ Adhere medium; and (c) a HIC step, wherein the HIC step comprises a medium comprising a 100 nm pore size polymethacrylate-based material bonded with C6 groups (hexyl), e.g., a Toyopearl® Hexyl-650 medium.

In some embodiments, provided is a purification platform comprising, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/AEX chromatography step, wherein the MM-HIC/AEX chromatography step comprises a medium comprising a multimodal strong anion exchanger using a N-benzyl-N-methyl ethanolamine ligand, e.g., a Capto™ Adhere medium; and (c) a HIC step, wherein the HIC step comprises a medium comprising a cross-linked poly(styrene-divinylbenzene) POROS™-based bead with aromatic hydrophobic benzyl ligands, e.g., Poros™ Benzyl Ultra medium.

In some embodiments, provided is a purification platform comprising, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/CEX chromatography step, wherein the MM-HIC/CEX chromatography step comprises a medium comprising a multimodal weak cation exchanger using a N-benzoyl-homocysteine ligand, e.g., a Capto™ MMC medium; and (c) a HIC step, wherein the HIC step comprises a medium comprising a cross-linked 6% agarose bead modified with aromatic phenyl groups via an uncharged and chemically-stable ether linkage, e.g., a Phenyl SFF medium, such as Phenyl SFF HS or Phenyl SFF LS.

In some embodiments, provided is a purification platform comprising, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/CEX chromatography step, wherein the MM-HIC/CEX chromatography step comprises a medium comprising a multimodal weak cation exchanger using a N-benzoyl-homocysteine ligand, e.g., a Capto™ MMC medium; and (c) a HIC step, wherein the HIC step comprises a medium comprising a 100 nm pore size polymethacrylate-based material bonded with C6 groups (hexyl), e.g., a Toyopearl® Hexyl-650 medium.

In some embodiments, provided is a purification platform comprising, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/CEX chromatography step, wherein the MM-HIC/CEX chromatography step comprises a medium comprising a multimodal weak cation exchanger using a N-benzoyl-homocysteine ligand, e.g., a Capto™ MMC medium; and (c) a HIC step, wherein the HIC step comprises a medium comprising a cross-linked poly(styrene-divinylbenzene) POROS™-based bead with aromatic hydrophobic benzyl ligands, e.g., Poros™ Benzyl Ultra medium. In some embodiments, the purification platform further comprises a depth filtration step, positioned at any position.

In some embodiments, the purification platform comprises, in order: (a) a capture step; (b) a MM-HIC/AEX chromatography step; (c) a HIC step; (d) a virus filtration step; and (e) a UF/DF step. In some embodiments, the capture step comprises processing via a protein A-based affinity medium, e.g., a MabSelect SuRe™ or MabSelect™ PrimaA medium. In some embodiments, the capture step comprises a bind-and-elute mode affinity chromatography step. In some embodiments, the MM-HIC/AEX chromatography step comprises a medium comprising a multimodal strong anion exchanger using a N-benzyl-N-methyl ethanolamine ligand, e.g., a Capto™ Adhere medium. In some embodiments, the HIC step comprises a medium comprising a cross-linked 6% agarose bead modified with aromatic phenyl groups via an uncharged and chemically-stable ether linkage, wherein the medium comprises approximately 40-45 μmol phenyl/mL medium, e.g., a Phenyl SFF HS medium. In some embodiments, the HIC step comprises a medium comprising a 100 nm pore size polymethacrylate-based material bonded with C6 groups (hexyl), e.g., a Toyopearl® Hexyl-650 medium. In some embodiments, the HIC step comprises a medium comprising a cross-linked poly(styrene-divinylbenzene) POROS™-based bead with aromatic hydrophobic benzyl ligands, e.g., Poros™ Benzyl Ultra.

In some embodiments, provided is a purification platform comprising, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/AEX chromatography step, wherein the MM-HIC/AEX chromatography step comprises a medium comprising a multimodal strong anion exchanger using a N-benzyl-N-methyl ethanolamine ligand, e.g., a Capto™ Adhere medium; (c) a HIC step, wherein the HIC step comprises a medium comprising a cross-linked 6% agarose bead modified with aromatic phenyl groups via an uncharged and chemically-stable ether linkage, and wherein the medium comprises approximately 40-45 μmol phenyl/mL medium, e.g., a Phenyl SFF HS medium; (d) a virus filtration step; and (e) a UF/DF step.

In some embodiments, provided is a purification platform comprising, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/AEX chromatography step, wherein the MM-HIC/AEX chromatography step comprises a medium comprising a multimodal strong anion exchanger using a N-benzyl-N-methyl ethanolamine ligand, e.g., a Capto™ Adhere medium; and (c) a HIC step, wherein the HIC step comprises a medium comprising a 100 nm pore size polymethacrylate-based material bonded with C6 groups (hexyl), e.g., a Toyopearl® Hexyl-650 medium; (d) a virus filtration step; and (e) a UF/DF step.

In some embodiments, provided is a purification platform comprising, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/AEX chromatography step, wherein the MM-HIC/AEX chromatography step comprises a medium comprising a multimodal strong anion exchanger using a N-benzyl-N-methyl ethanolamine ligand, e.g., a Capto™ Adhere medium; and (c) a HIC step, wherein the HIC step comprises a medium comprising a cross-linked poly(styrene-divinylbenzene) POROS™-based bead with aromatic hydrophobic benzyl ligands, e.g., Poros™ Benzyl Ultra medium; (d) a virus filtration step; and (e) a UF/DF step.

In some embodiments, provided is a purification platform comprising, in order: (a) a capture step; (b) a MM-HIC/AEX chromatography step using a depth filtration step as a load filtration step; and (c) a HIC step. In some embodiments, the capture step comprises a bind-and-elute mode affinity chromatography step. In some embodiments, the capture step comprises a protein A-based affinity medium comprising a rigid, high-flow agarose matrix and alkali-stabilized protein A-derived ligand, wherein amino acids particularly sensitive to alkali were substituted with more stable residue in an alkali environment, e.g. a MabSelect SuRe™ medium. In some embodiments, the capture step comprises a protein A-based affinity medium comprising a rigid, high-flow agarose matrix and a protein A-derived ligand having alkaline stability, e.g., a MabSelect™ PrismA medium. In some embodiments, the MM-HIC/AEX chromatography step comprises a medium comprising a multimodal strong anion exchanger using a N-benzyl-N-methyl ethanolamine ligand, e.g., a Capto™ Adhere medium. In some embodiments, the depth filtration step comprises a depth filter comprising a silica, such as a silica filter aid, and a polyacrylic fiber, e.g., a X0SP depth filter, a C0SP depth filter, or a D0SP depth filter. In some embodiments, the depth filtration step comprises a depth filter comprising a hydrogel Q (quaternary amine, also referred to as a quaternary ammonium)-functionalized non-woven material, and a multizone microporous membrane, e.g., EMPHAZE™ AEX depth filter. In some embodiments, the HIC step is a flow-through mode HIC step. In some embodiments, the HIC step comprises a medium comprising a 100 nm pore size polymethacrylate-based material bonded with C6 groups (hexyl), e.g., a Toyopearl® Hexyl-650 medium. In some embodiments, the HIC step comprises a medium comprising a cross-linked poly(styrene-divinylbenzene) POROS™-based bead with aromatic hydrophobic benzyl ligands, e.g., Poros™ Benzyl Ultra. In some embodiments, the HIC step comprises a medium comprising a cross-linked 6% agarose bead modified with aromatic phenyl groups via an uncharged and chemically-stable ether linkage, wherein the medium comprises approximately 40-45 μmol phenyl/mL medium, e.g., a Phenyl SFF HS medium. In some embodiments, the HIC step comprises a pH adjustment step, wherein the pH of the input material is pH adjusted to a pH of about 4.5 to about 6. In some embodiments, the HIC step is a low salt HIC step, such as no salt, such as a HIC conditioning salt, is added prior to loading a material on the HIC membrane or the HIC column.

In some embodiments, provided is a purification platform comprising, in order: (a) a capture step, wherein the capture step comprises a protein A-based affinity medium comprising a rigid, high-flow agarose matrix and a protein A-derived ligand having alkaline stability, e.g., a MabSelect™ PrismA medium; (b) a MM-HIC/AEX chromatography step using a depth filtration step as a load filtration step, wherein the MM-HIC/AEX chromatography step comprises a medium comprising a multimodal strong anion exchanger using a N-benzyl-N-methyl ethanolamine ligand, e.g., a Capto™ Adhere medium, and wherein the depth filtration step comprises a depth filter comprising a silica, such as a silica filter aid, and a polyacrylic fiber, e.g., a X0SP depth filter, a C0SP depth filter, or a D0SP depth filter; and (c) a HIC step, wherein the HIC step is a flow-through mode HIC step, and wherein the HIC step comprises a medium comprising a 100 nm pore size polymethacrylate-based material bonded with C6 groups (hexyl), e.g., a Toyopearl® Hexyl-650 medium. In some embodiments, the HIC step comprises a pH adjustment step, wherein the pH of the input material is pH adjusted to a pH of about 4.5 to about 5.5, such as a about 5.0. In some embodiments, the HIC step is a low salt HIC step, such as no salt, such as a HIC conditioning salt, is added prior to loading a material on the HIC membrane or the HIC column.

In some embodiments, provided is a purification platform comprising, in order: (a) a capture step, wherein the capture step comprises a protein A-based affinity medium comprising a rigid, high-flow agarose matrix and a protein A-derived ligand having alkaline stability, e.g., a MabSelect™ PrismA medium; (b) a MM-HIC/AEX chromatography step using a depth filtration step as a load filtration step, wherein the MM-HIC/AEX chromatography step comprises a medium comprising a multimodal strong anion exchanger using a N-benzyl-N-methyl ethanolamine ligand, e.g., a Capto™ Adhere medium, and wherein the depth filtration step comprises a depth filter comprising a silica, such as a silica filter aid, and a polyacrylic fiber, e.g., a X0SP depth filter, a C0SP depth filter, or a D0SP depth filter; and (c) a HIC step, wherein the HIC step is a flow-through mode HIC step, and wherein the HIC step comprises a medium comprising a cross-linked poly(styrene-divinylbenzene) POROS™-based bead with aromatic hydrophobic benzyl ligands, e.g., Poros™ Benzyl Ultra. In some embodiments, the HIC step comprises a pH adjustment step, wherein the pH of the input material is pH adjusted to a pH of about 4.5 to about 5.5, such as a about 5.0. In some embodiments, the HIC step is a low salt HIC step such as no salt, such as a HIC conditioning salt, is added prior to loading a material on the HIC membrane or the HIC column.

In some embodiments, provided is a purification platform comprising, in order: (a) a capture step, wherein the capture step comprises a protein A-based affinity medium comprising a rigid, high-flow agarose matrix and alkali-stabilized protein A-derived ligand, wherein amino acids particularly sensitive to alkali were substituted with more stable residue in an alkali environment, e.g. a MabSelect SuRe™ medium; (b) a MM-HIC/AEX chromatography step using a depth filtration step as a load filtration step, wherein the MM-HIC/AEX chromatography step comprises a medium comprising a multimodal strong anion exchanger using a N-benzyl-N-methyl ethanolamine ligand, e.g., a Capto™ Adhere medium, and wherein the depth filtration step comprises a depth filter comprising a hydrogel Q (quaternary amine, also referred to as a quaternary ammonium)-functionalized non-woven material, and a multizone microporous membrane, e.g., EMPHAZE™ AEX depth filter; and (c) a HIC step, wherein the HIC step is a flow-through mode HIC step, and wherein the HIC step comprises a medium comprising a cross-linked 6% agarose bead modified with aromatic phenyl groups via an uncharged and chemically-stable ether linkage, wherein the medium comprises approximately 40-45 μmol phenyl/mL medium, e.g., a Phenyl SFF HS medium. In some embodiments, the HIC step comprises a pH adjustment step, wherein the pH of the input material is pH adjusted to a pH of about 5.0 to about 6.0, such as a about 5.5. In some embodiments, the HIC step is a low salt HIC step, such as no salt, such as a HIC conditioning salt, is added prior to loading a material on the HIC membrane or the HIC column.

In some embodiments, provided herein is a purification platform comprising a capture step; one or more ion exchange (IEX) chromatography steps; and a hydrophobic interaction chromatography (HIC) step. In some embodiments, the IEX chromatography step is a CEX chromatography step. In some embodiments, the purification platform further comprises a virus filtration step. In some embodiments, the purification platform further comprises a UF/DF step. In some embodiments, the purification platform further comprises a depth filtration step. In some embodiments, the purification platform further comprises a HIC step. Exemplary purification platforms encompassed within the described purification platforms is shown in FIG. 1C and are described in more detail below.

In some embodiments, the purification platform comprises, in order: (a) a capture step; (b) a CEX chromatography step; (c) a HIC step; (d) a virus filtration step; and (e) a UF/DF step. In some embodiments, the capture step comprises a protein A-based affinity medium, e.g., a MabSelect SuRe™ or MabSelect™ PrimaA medium. In some embodiments, the capture step comprises a bind-and-elute mode affinity chromatography step. In some embodiments, the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., a Poros™ XS medium. In some embodiments, the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., Poros™ 50HS. In some embodiments, the HIC step comprises a medium comprising a cross-linked 6% agarose bead modified with aromatic phenyl groups via an uncharged and chemically-stable ether linkage, wherein the medium comprises approximately 40-45 μmol phenyl/mL medium, e.g., a Phenyl SFF HS medium. In some embodiments, the HIC step comprises a medium comprising a 100 nm pore size polymethacrylate-based material bonded with C6 groups (hexyl), e.g., a Toyopearl® Hexyl-650 medium. In some embodiments, the HIC step comprises a medium comprising a cross-linked poly(styrene-divinylbenzene) POROS™-based bead with aromatic hydrophobic benzyl ligands, e.g., Poros™ Benzyl Ultra.

In some embodiments, the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a CEX chromatography step, wherein the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., a Poros™ XS medium; (c) a HIC step, wherein the HIC step comprises a medium comprising a cross-linked 6% agarose bead modified with aromatic phenyl groups via an uncharged and chemically-stable ether linkage, wherein the medium comprises approximately 40-45 μmol phenyl/mL medium, e.g., a Phenyl SFF HS medium; (d) a virus filtration step; and (e) a UF/DF step.

In some embodiments, the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a CEX chromatography step, wherein the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., a Poros™ XS medium; (c) a HIC step, wherein the HIC step comprises a medium comprising a 100 nm pore size polymethacrylate-based material bonded with C6 groups (hexyl), e.g., a Toyopearl® Hexyl-650 medium; (d) a virus filtration step; and (e) a UF/DF step.

In some embodiments, the purification platform comprises, in order: (a) a capture step, wherein the capture step comprises a bind-and-elute mode affinity chromatography step; (b) a CEX chromatography step, wherein the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., a Poros™ XS medium; (c) a HIC step, wherein the HIC step comprises a medium comprising a cross-linked poly(styrene-divinylbenzene) POROS™-based bead with aromatic hydrophobic benzyl ligands, e.g., a Poros™ Benzyl Ultra medium; (d) a virus filtration step; and (e) a UF/DF step.

In some embodiments, the purification platform comprises, in order: (a) a capture step, wherein the capture step comprises a bind-and-elute mode affinity chromatography step; (b) a CEX chromatography step, wherein the CEX chromatography step comprises a CEX chromatography medium comprising rigid polymeric resin particles comprising cross-linked poly[styrene-divinylbenzene]having a polyhydroxyl surface coating further functionalization with sulphopropyl (SP) strong cation exchange groups, e.g., a Poros™ 50HS medium; (c) a HIC step, wherein the HIC step comprises a medium comprising a cross-linked 6% agarose bead modified with aromatic phenyl groups via an uncharged and chemically-stable ether linkage, wherein the medium comprises approximately 40-45 μmol phenyl/mL medium, e.g., a Phenyl SFF HS medium; (d) a virus filtration step; and (e) a UF/DF step.

In some embodiments, the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a CEX chromatography step, wherein the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., a Poros™ 50HS medium; (c) a HIC step, wherein the HIC step comprises a medium comprising a 100 nm pore size polymethacrylate-based material bonded with C6 groups (hexyl), e.g., a Toyopearl® Hexyl-650 medium; (d) a virus filtration step; and (e) a UF/DF step.

In some embodiments, the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a CEX chromatography step, wherein the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., a Poros™ 50HS medium; (c) a HIC step, wherein the HIC step comprises a medium comprising a cross-linked poly(styrene-divinylbenzene) POROS™-based bead with aromatic hydrophobic benzyl ligands, e.g., a Poros™ Benzyl Ultra medium; (d) a virus filtration step; and (e) a UF/DF step. In some embodiments, the purification platform further comprises a depth filtration step, positioned at any position

In some embodiments, provided is a purification platform comprising: one or more ion exchange (IEX) chromatography steps; a hydrophobic interaction chromatography (HIC) step; and a depth filtration step. In some embodiments, the purification platform further comprises a virus filtration step and/or a UF/DF step. In some embodiments, the depth filtration step and/or the HIC step is directly before the virus filtration step of the UF/DF step. In some embodiments, the depth filtration step and/or the HIC step is sequential with or directly after the IEX chromatography step. Exemplary purification platforms encompassed within the described purification platforms is shown in FIG. 1D and described in more detail below.

In some embodiments, the purification platform comprises, in order: (a) a CEX chromatography step; (b) a HIC step; (c) a MMIEX chromatography step; (d) an AEX chromatography step; (e) a depth filter step; and (f) a UF/DF step. In some embodiments, the CEX chromatography step comprises a CEX chromatography medium comprising cross-linked 6% agarose beads having dextran chains covalently coupled to the agarose matrix that are modified with sulphopropyl (SP) strong cation exchange groups, e.g., a SP Sepharose® XL (SPXL) medium or a Streamline™ SPXL medium. In some embodiments, the HIC step comprises a HIC medium comprising propyl groups covalently linked to nitrogens on polyethylenimine (PEI) ligands attached to a substrate, e.g., a Bakerbond WP HI-Propyl™ medium. In some embodiments, the MMIEX chromatography step comprises a MMIEX chromatography medium comprising silica gel solid phase particles comprising a mixed mode anion/cation exchanger, e.g., a Bakerbond ABx™ medium. In some embodiments, the AEX chromatography step comprises an AEX chromatography medium comprising a cross-linked 6% agarose bead having quaternary ammonium (Q) strong anion exchange groups, e.g., a Q Sepharose® Fast Flow (QSFF) medium. In some embodiments, the depth filtration step is comprises a depth filter comprising a silica, such as a silica filter aid, and a polyacrylic fiber, e.g., a X0SP depth filter, a C0SP depth filter, or a D0SP depth filter.

In some embodiments, the purification platform comprises, in order: (a) a CEX chromatography step, wherein the CEX chromatography step comprises a CEX chromatography medium comprising cross-linked 6% agarose beads having dextran chains covalently coupled to the agarose matrix that are modified with sulphopropyl (SP) strong cation exchange groups, e.g., a SP Sepharose® XL (SPXL) medium or a Streamline™ SPXL medium; (b) a HIC step, wherein the HIC step comprises a HIC medium comprising propyl groups covalently linked to nitrogens on polyethylenimine (PEI) ligands attached to a substrate, e.g., a Bakerbond WP HI-Propyl™ medium; (c) a MMIEX chromatography step, wherein the MMIEX chromatography step comprises a MMIEX chromatography medium comprising silica gel solid phase particles comprising a mixed mode anion/cation exchanger, e.g., a Bakerbond ABx™ medium; (d) a AEX chromatography step, wherein the AEX chromatography step comprises an AEX chromatography medium comprising a cross-linked 6% agarose bead having quaternary ammonium (Q) strong anion exchange groups, e.g., a Q Sepharose® Fast Flow (QSFF) medium; (e) a depth filter step, wherein the depth filtration step is comprises a depth filter comprising a silica, such as a silica filter aid, and a polyacrylic fiber, e.g., a X0SP depth filter, a C0SP depth filter, or a D0SP depth filter; and (f) a UF/DF step. In some embodiments, the purification platform further comprises a depth filtration step, positioned at any position, wherein the depth filtration step comprises a depth filter comprising a silica, such as a silica filter aid, and a polyacrylic fiber, e.g., a X0SP depth filter, a C0SP depth filter, or a D0SP depth filter, and wherein the depth filtration step is configured to be performed at, e.g., the input material has, a pH of about 6 to about 7, such as about 6.5. In some embodiments, the purification platform further comprises a depth filtration step, positioned at any position, wherein the depth filtration step comprises a depth filter comprising a hydrogel Q (quaternary amine, also referred to as a quaternary ammonium)-functionalized non-woven material, and a multizone microporous membrane, e.g., an EMPHAZE™ AEX depth filter, and wherein the depth filtration step is configured to be performed at, e.g., the input material has, a pH of about 8.5 to about 9.5, such as about 9.1. In some embodiments, the purification platform further comprises a HIC step, positioned at any position, wherein the HIC step comprises a HIC medium, such as a HIC membrane, comprising a phenyl moiety conjugated to a stabilized reinforced cellulose filter, e.g., a Sartobind® Phenyl medium, wherein the HIC step is configured to be performed at, e.g., the input material has, a pH of about 6 to about 7, such as about 6.5.

In some embodiments, provided is a purification platform comprising: (a) a capture step; (b) one or more ion exchange (IEX) chromatography steps, such as a CEX chromatography step; (c) a multimodal hydrophobic interaction/ion exchange (MM-HIC/IEX) chromatography steps; and (d) one or both of: (i) a hydrophobic interaction chromatography (HIC) step; and (ii) a depth filtration step. In some embodiments, the purification platform further comprises a virus filtration step. In some embodiments, the purification platform further comprises a UF/DF step. Exemplary purification platforms encompassed within the described purification platforms is shown in FIG. 1E. In some embodiments, the capture step comprises a protein A-based affinity medium, e.g., a MabSelect SuRe™ or MabSelect™ PrimaA medium. In some embodiments, the capture step comprises a bind-and-elute mode affinity chromatography step. In some embodiments, the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., a Poros™ XS medium. In some embodiments, the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., Poros™ 50HS. In some embodiments, the MM-HIC/AEX chromatography step comprises a medium comprising a multimodal strong anion exchanger using a N-benzyl-N-methyl ethanolamine ligand, e.g., a Capto™ Adhere medium. In some embodiments, the purification platform further comprises a depth filtration step, positioned at any position.

### II. Method of Using Purification Platforms

In some aspects, the present disclosure provides methods of using the purification platforms described herein. In some embodiments, the method comprises subjecting a sample comprising a target to a purification platform described herein.

In some embodiments, the methods described herein are capable of reducing a hydrolytic enzyme activity rate of a composition obtained from a purification platform. In some embodiments, the hydrolytic enzyme activity rate represents the activity rate of one or more hydrolytic enzymes, such as one or more different hydrolytic enzymes. As discussed in other sections herein, in some embodiments, the hydrolytic enzyme activity rate is a surrogate measurement of the activity of one or more enzymes in the composition. In some embodiments, the hydrolytic enzyme activity rate is measured via a surrogate substrate. In some embodiments, the hydrolytic enzyme activity rate is assessed by measuring the hydrolytic product of one or more hydrolytic enzymes. In some aspects, the reduction in the hydrolytic enzyme activity rate is at least about 20%, such as at least about any of 25%, 30%, 35%, 40%, 45%, 50%, 55%, 60%, 65%, 70%, 85%, 90%, or 95%.

In some aspects, provided is a method comprising subjecting a sample to a purification platform for purifying a target from a sample, wherein the purification platform comprises: a capture step; one or more ion exchange (IEX) chromatography steps; and a depth filtration step. In some embodiments, the purification platform further comprises a virus filtration step and/or a UF/DF step. In some embodiments, the depth filtration step is directly prior to the virus filtration step or the UF/DF step. In some embodiments, the depth filtration step is sequential with or directly after an IEX chromatography step. In some embodiments, the depth filtration step comprising a X0SP depth filter on an EMPHAZE™ depth filter. In some embodiments, the purification platform further comprises a HIC step.

In some embodiments, the method comprises subjecting a sample to a purification platform for purifying a target from a sample, wherein the purification platform comprises, in order: (a) a capture step; (b) a CEX chromatography step; (c) an AEX chromatography step; (d) a depth filtration step; (e) a virus filtration step; and (f) a UF/DF step. In some embodiments, the capture step comprises processing via a protein A-based affinity medium, e.g., a MabSelect SuRe™ or MabSelect™ PrimaA medium. In some embodiments, the capture step comprises a bind-and-elute mode affinity chromatography step. In some embodiments, the CEX chromatography step comprises a CEX chromatography medium comprises cross-linked 6% agarose beads having sulphopropyl (SP) strong cation exchange groups, e.g., a SP Sepharose® Fase Flow (SPSFF) medium. In some embodiments, the AEX chromatography step comprises an AEX chromatography medium comprising a cross-linked 6% agarose bead having quaternary ammonium (Q) strong anion exchange groups, e.g., a Q Sepharose® Fast Flow (QSFF) medium. In some embodiments, the depth filtration step is comprises a depth filter comprising a silica, such as a silica filter aid, and/or a polyacrylic fiber, e.g., a X0SP depth filter, a C0SP depth filter, or a D0SP depth filter.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a CEX chromatography step, wherein the CEX chromatography step comprises a CEX chromatography medium comprises cross-linked 6% agarose beads having sulphopropyl (SP) strong cation exchange groups, e.g., a SP Sepharose® Fase Flow (SPSFF) medium; (c) an AEX chromatography step, wherein the AEX chromatography step comprises an AEX chromatography medium comprising a cross-linked 6% agarose bead having quaternary ammonium (Q) strong anion exchange groups, e.g., a Q Sepharose® Fast Flow (QSFF) medium; (d) a depth filtration step, wherein the depth filtration step comprises a depth filter comprising a silica, such as a silica filter aid, and/or a polyacrylic fiber, e.g., a X0SP depth filter, a C0SP depth filter, or a D0SP depth filter; a virus filtration step; and a UF/DF step. In some embodiments, the purification platform further comprises a depth filtration step, positioned at any position, wherein the depth filtration step comprises a depth filter comprising a silica, such as a silica filter aid, and/or a polyacrylic fiber, e.g., a X0SP depth filter, a C0SP depth filter, or a D0SP depth filter, and wherein the depth filtration step is configured to be performed at, e.g., the input material has, a pH of about 5 to about 6.5, such as about 5 to about 5.5 or about 5.8 to about 6.2. In some embodiments, the purification platform further comprises a depth filtration step, positioned at any position, wherein the depth filtration step comprises a depth filter comprising a hydrogel Q (quaternary amine, also referred to as a quaternary ammonium)-functionalized non-woven material, and a multizone microporous membrane, e.g., an EMPHAZE™ AEX depth filter, and wherein the depth filtration step is configured to be performed at, e.g., the input material has, a pH of about 7.5 to about 8.5, such as about 8. In some embodiments, the purification platform further comprises a HIC step, positioned at any position, wherein the HIC step comprises a HIC medium, such as a HIC membrane, comprising a phenyl moiety conjugated to a stabilized reinforced cellulose filter, e.g., a Sartobind® Phenyl medium, wherein the HIC step is configured to be performed at, e.g., the input material has, a pH of about 5 to about 6, such as about 5.5.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a CEX chromatography step, wherein the CEX chromatography step comprises a CEX chromatography medium comprises cross-linked 6% agarose beads having sulphopropyl (SP) strong cation exchange groups, e.g., a SP Sepharose® Fase Flow (SPSFF) medium; (c) an AEX chromatography step, wherein the AEX chromatography step comprises an AEX chromatography medium comprising a cross-linked 6% agarose bead having quaternary ammonium (Q) strong anion exchange groups, e.g., a Q Sepharose® Fast Flow (QSFF) medium; (d) a depth filtration step, wherein the depth filtration step comprises a depth filter comprising a hydrogel Q (quaternary amine, also referred to as a quaternary ammonium)-functionalized non-woven material, and a multizone microporous membrane, e.g., an EMPHAZE™ AEX depth filter; a virus filtration step; and a UF/DF step. In some embodiments, the purification platform further comprises a depth filtration step, positioned at any position, wherein the depth filtration step comprises a depth filter comprising a silica, such as a silica filter aid, and a polyacrylic fiber, e.g., a X0SP depth filter, a C0SP depth filter, or a D0SP depth filter, and wherein the depth filtration step is configured to be performed at, e.g., the input material has, a pH of about 5 to about 6, such as about 5.5. In some embodiments, the purification platform further comprises a depth filtration step, positioned at any position, wherein the depth filtration step comprises a depth filter comprising a hydrogel Q (quaternary amine, also referred to as a quaternary ammonium)-functionalized non-woven material, and a multizone microporous membrane, e.g., an EMPHAZE™ AEX depth filter, and wherein the depth filtration step is configured to be performed at, e.g., the input material has, a pH of about 7.5 to about 8.5, such as about 8. In some embodiments, the purification platform further comprises a HIC step, positioned at any position, wherein the HIC step comprises a HIC medium, such as a HIC membrane, comprising a phenyl moiety conjugated to a stabilized reinforced cellulose filter, e.g., a Sartobind® Phenyl medium, wherein the HIC step is configured to be performed at, e.g., the input material has, a pH of about 5 to about 6, such as about 5.5.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a CEX chromatography step, wherein the CEX chromatography step comprises a CEX chromatography medium comprises cross-linked 6% agarose beads having sulphopropyl (SP) strong cation exchange groups, e.g., a SP Sepharose® Fase Flow (SPSFF) medium; (c) an AEX chromatography step, wherein the AEX chromatography step comprises an AEX chromatography medium comprising a cross-linked 6% agarose bead having quaternary ammonium (Q) strong anion exchange groups, e.g., a Q Sepharose® Fast Flow (QSFF) medium; (d) a HIC step, wherein the HIC step comprises a HIC medium comprising a phenyl moiety conjugated to a stabilized reinforced cellulose filter, e.g., Sartobind® Phenyl; a virus filtration step; and a UF/DF step. In some embodiments, the purification platform further comprises a depth filtration step, positioned at any position, wherein the depth filtration step comprises a depth filter comprising a silica, such as a silica filter aid, and a polyacrylic fiber, e.g., a X0SP depth filter, a C0SP depth filter, or a D0SP depth filter, and wherein the depth filtration step is configured to be performed at, e.g., the input material has, a pH of about 5 to about 6, such as about 5.5. In some embodiments, the purification platform further comprises a depth filtration step, positioned at any position, wherein the depth filtration step comprises a depth filter comprising a hydrogel Q (quaternary amine, also referred to as a quaternary ammonium)-functionalized non-woven material, and a multizone microporous membrane, e.g., an EMPHAZE™ AEX depth filter, and wherein the depth filtration step is configured to be performed at, e.g., the input material has, a pH of about 7.5 to about 8.5, such as about 8. In some embodiments, the purification platform further comprises a HIC step, positioned at any position, wherein the HIC step comprises a HIC medium, such as a HIC membrane, comprising a phenyl moiety conjugated to a stabilized reinforced cellulose filter, e.g., a Sartobind® Phenyl medium, wherein the HIC step is configured to be performed at, e.g., the input material has, a pH of about 5 to about 6, such as about 5.5.

In some aspects, provided is a method comprising subjecting a sample to a purification platform for purifying a target from a sample, wherein the purification platform comprises: capture step; a multimodal hydrophobic interaction/ion exchange (MM-HIC/IEX) chromatography steps; and a hydrophobic interaction chromatography (HIC) step. In some embodiments, the purification platform further comprises a virus filtration step. In some embodiments, the purification platform further comprises a UF/DF step. Exemplary purification platforms encompassed within the described purification platforms is shown in FIG. 1B and described in more detail below.

In some embodiments, the method comprises subjecting a sample to a purification platform for purifying a target from a sample, wherein the purification platform comprises, in order: (a) a capture step; (b) a MM-HIC/IEX chromatography step; and (c) a HIC step. In some embodiments, the capture step comprises a protein A-based affinity medium, e.g., a MabSelect SuRe™ or MabSelect™ PrimaA medium. In some embodiments, the capture step comprises a bind-and-elute mode affinity chromatography step. In some embodiments, the MM-HIC/IEX chromatography step comprises a MM-HIC/AEX chromatography step. In some embodiments, the MM-HIC/AEX chromatography step comprises a medium comprising a multimodal strong anion exchanger using a N-benzyl-N-methyl ethanolamine ligand, e.g., a Capto™ Adhere medium. In some embodiments, the MM-HIC/IEX chromatography step is a MM-HIC/CEX chromatography step. In some embodiments, the MM-HIC/CEX chromatography step comprises a medium comprising a multimodal weak cation exchanger using a N-benzoyl-homocysteine ligand, e.g., a Capto™ MMC medium. In some embodiments, the HIC step comprises a medium comprising a cross-linked 6% agarose bead modified with aromatic phenyl groups via an uncharged and chemically-stable ether linkage, e.g., a Phenyl SFF medium, such as Phenyl SFF HS or Phenyl SFF LS. In some embodiments, the HIC step comprises a medium comprising a 100 nm pore size polymethacrylate-based material bonded with C6 groups (hexyl), e.g., a Toyopearl® Hexyl-650 medium, such as Toyopearl® Hexyl-650C. In some embodiments, the HIC step comprises a medium comprising a cross-linked poly(styrene-divinylbenzene) POROS™-based bead with aromatic hydrophobic benzyl ligands, e.g., Poros™ Benzyl Ultra.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/AEX chromatography step, wherein the MM-HIC/AEX chromatography step comprises a medium comprising a multimodal strong anion exchanger using a N-benzyl-N-methyl ethanolamine ligand, e.g., a Capto™ Adhere medium; and (c) a HIC step, wherein the HIC step comprises a medium comprising a cross-linked 6% agarose bead modified with aromatic phenyl groups via an uncharged and chemically-stable ether linkage, e.g., a Phenyl SFF medium, such as Phenyl SFF HS or Phenyl SFF LS.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/AEX chromatography step, wherein the MM-HIC/AEX chromatography step comprises a medium comprising a multimodal strong anion exchanger using a N-benzyl-N-methyl ethanolamine ligand, e.g., a Capto™ Adhere medium; and (c) a HIC step, wherein the HIC step comprises a medium comprising a 100 nm pore size polymethacrylate-based material bonded with C6 groups (hexyl), e.g., a Toyopearl® Hexyl-650 medium.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/AEX chromatography step, wherein the MM-HIC/AEX chromatography step comprises a medium comprising a multimodal strong anion exchanger using a N-benzyl-N-methyl ethanolamine ligand, e.g., a Capto™ Adhere medium; and (c) a HIC step, wherein the HIC step comprises a medium comprising a cross-linked poly(styrene-divinylbenzene) POROS™-based bead with aromatic hydrophobic benzyl ligands, e.g., Poros™ Benzyl Ultra medium.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/CEX chromatography step, wherein the MM-HIC/CEX chromatography step comprises a medium comprising a multimodal weak cation exchanger using a N-benzoyl-homocysteine ligand, e.g., a Capto™ MMC medium; and (c) a HIC step, wherein the HIC step comprises a medium comprising a cross-linked 6% agarose bead modified with aromatic phenyl groups via an uncharged and chemically-stable ether linkage, e.g., a Phenyl SFF medium, such as Phenyl SFF HS or Phenyl SFF LS.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/CEX chromatography step, wherein the MM-HIC/CEX chromatography step comprises a medium comprising a multimodal weak cation exchanger using a N-benzoyl-homocysteine ligand, e.g., a Capto™ MMC medium; and (c) a HIC step, wherein the HIC step comprises a medium comprising a 100 nm pore size polymethacrylate-based material bonded with C6 groups (hexyl), e.g., a Toyopearl® Hexyl-650 medium.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/CEX chromatography step, wherein the MM-HIC/CEX chromatography step comprises a medium comprising a multimodal weak cation exchanger using a N-benzoyl-homocysteine ligand, e.g., a Capto™ MMC medium; and (c) a HIC step, wherein the HIC step comprises a medium comprising a cross-linked poly(styrene-divinylbenzene) POROS™-based bead with aromatic hydrophobic benzyl ligands, e.g., Poros™ Benzyl Ultra medium. In some embodiments, the purification platform further comprises a depth filtration step, positioned at any position.

In some embodiments, the method comprises subjecting a sample to a purification platform for purifying a target from a sample, wherein the purification platform comprises, in order: (a) a capture step; (b) a MM-HIC/AEX chromatography step; (c) a HIC step; (d) a virus filtration step; and (e) a UF/DF step. In some embodiments, the capture step comprises processing via a protein A-based affinity medium, e.g., a MabSelect SuRe™ or MabSelect™ PrimaA medium. In some embodiments, the capture step comprises a bind-and-elute mode affinity chromatography step. In some embodiments, the MM-HIC/AEX chromatography step comprises a medium comprising a multimodal strong anion exchanger using a N-benzyl-N-methyl ethanolamine ligand, e.g., a Capto™ Adhere medium. In some embodiments, the HIC step comprises a medium comprising a cross-linked 6% agarose bead modified with aromatic phenyl groups via an uncharged and chemically-stable ether linkage, wherein the medium comprises approximately 40-45 μmol phenyl/mL medium, e.g., a Phenyl SFF HS medium. In some embodiments, the HIC step comprises a medium comprising a 100 nm pore size polymethacrylate-based material bonded with C6 groups (hexyl), e.g., a Toyopearl® Hexyl-650 medium. In some embodiments, the HIC step comprises a medium comprising a cross-linked poly(styrene-divinylbenzene) POROS™-based bead with aromatic hydrophobic benzyl ligands, e.g., Poros™ Benzyl Ultra.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/AEX chromatography step, wherein the MM-HIC/AEX chromatography step comprises a medium comprising a multimodal strong anion exchanger using a N-benzyl-N-methyl ethanolamine ligand, e.g., a Capto™ Adhere medium; (c) a HIC step, wherein the HIC step comprises a medium comprising a cross-linked 6% agarose bead modified with aromatic phenyl groups via an uncharged and chemically-stable ether linkage, and wherein the medium comprises approximately 40-45 μmol phenyl/mL medium, e.g., a Phenyl SFF HS medium; (d) a virus filtration step; and (e) a UF/DF step.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/AEX chromatography step, wherein the MM-HIC/AEX chromatography step comprises a medium comprising a multimodal strong anion exchanger using a N-benzyl-N-methyl ethanolamine ligand, e.g., a Capto™ Adhere medium; and (c) a HIC step, wherein the HIC step comprises a medium comprising a 100 nm pore size polymethacrylate-based material bonded with C6 groups (hexyl), e.g., a Toyopearl® Hexyl-650 medium; (d) a virus filtration step; and (e) a UF/DF step.

In some embodiments, the method comprises subjecting a sample to a purification platform for purifying a target from a sample, wherein the purification platform comprises, in order: (a) a capture step, wherein the capture step capture step comprises a protein A-based affinity medium, e.g., a MabSelect SuRe™ or MabSelect™ PrimaA medium, and wherein the capture step is configured to be a bind-and-elute affinity chromatography step; (b) a MM-HIC/AEX chromatography step, wherein the MM-HIC/AEX chromatography step comprises a medium comprising a multimodal strong anion exchanger using a N-benzyl-N-methyl ethanolamine ligand, e.g., a Capto™ Adhere medium; and (c) a HIC step, wherein the HIC step comprises a medium comprising a cross-linked poly(styrene-divinylbenzene) POROS™-based bead with aromatic hydrophobic benzyl ligands, e.g., Poros™ Benzyl Ultra medium; (d) a virus filtration step; and (e) a UF/DF step.

In some embodiments, the method comprises subjecting a sample to a purification platform for purifying a target from a sample, wherein the purification platform comprises, in order: (a) a capture step; (b) a MM-HIC/AEX chromatography step using a depth filtration step as a load filtration step; and (c) a HIC step. In some embodiments, the capture step comprises a bind-and-elute mode affinity chromatography step. In some embodiments, the capture step comprises a protein A-based affinity medium comprising a rigid, high-flow agarose matrix and alkali-stabilized protein A-derived ligand, wherein amino acids particularly sensitive to alkali were substituted with more stable residue in an alkali environment, e.g. a MabSelect SuRe™ medium. In some embodiments, the capture step comprises a protein A-based affinity medium comprising a rigid, high-flow agarose matrix and a protein A-derived ligand having alkaline stability, e.g., a MabSelect™PrismA medium. In some embodiments, the MM-HIC/AEX chromatography step comprises a medium comprising a multimodal strong anion exchanger using a N-benzyl-N-methyl ethanolamine ligand, e.g., a Capto™ Adhere medium. In some embodiments, the depth filtration step comprises a depth filter comprising a silica, such as a silica filter aid, and a polyacrylic fiber, e.g., a X0SP depth filter, a C0SP depth filter, or a D0SP depth filter. In some embodiments, the depth filtration step comprises a depth filter comprising a hydrogel Q (quaternary amine, also referred to as a quaternary ammonium)-functionalized non-woven material, and a multizone microporous membrane, e.g., EMPHAZE™ AEX depth filter. In some embodiments, the HIC step is a flow-through mode HIC step. In some embodiments, the HIC step comprises a medium comprising a 100 nm pore size polymethacrylate-based material bonded with C6 groups (hexyl), e.g., a Toyopearl® Hexyl-650 medium. In some embodiments, the HIC step comprises a medium comprising a cross-linked poly(styrene-divinylbenzene) POROS™-based bead with aromatic hydrophobic benzyl ligands, e.g., Poros™ Benzyl Ultra. In some embodiments, the HIC step comprises a medium comprising a cross-linked 6% agarose bead modified with aromatic phenyl groups via an uncharged and chemically-stable ether linkage, wherein the medium comprises approximately 40-45 μmol phenyl/mL medium, e.g., a Phenyl SFF HS medium. In some embodiments, the HIC step comprises a pH adjustment step, wherein the pH of the input material is pH adjusted to a pH of about 4.5 to about 6. In some embodiments, the HIC step is a low salt HIC step, such as no salt, such as a HIC conditioning salt, is added prior to loading a material on the HIC membrane or the HIC column.

In some embodiments, the method comprises subjecting a sample to a purification platform for purifying a target from a sample, wherein the purification platform comprises, in order: (a) a capture step, wherein the capture step comprises a protein A-based affinity medium comprising a rigid, high-flow agarose matrix and a protein A-derived ligand having alkaline stability, e.g., a MabSelect™ PrismA medium; (b) a MM-HIC/AEX chromatography step using a depth filtration step as a load filtration step, wherein the MM-HIC/AEX chromatography step comprises a medium comprising a multimodal strong anion exchanger using a N-benzyl-N-methyl ethanolamine ligand, e.g., a Capto™ Adhere medium, and wherein the depth filtration step comprises a depth filter comprising a silica, such as a silica filter aid, and a polyacrylic fiber, e.g., a X0SP depth filter, a C0SP depth filter, or a D0SP depth filter; and (c) a HIC step, wherein the HIC step is a flow-through mode HIC step, and wherein the HIC step comprises a medium comprising a 100 nm pore size polymethacrylate-based material bonded with C6 groups (hexyl), e.g., a Toyopearl® Hexyl-650 medium. In some embodiments, the HIC step comprises a pH adjustment step, wherein the pH of the input material is pH adjusted to a pH of about 4.5 to about 5.5, such as a about 5.0. In some embodiments, the HIC step is a low salt HIC step, such as no salt, such as a HIC conditioning salt, is added prior to loading a material on the HIC membrane or the HIC column.

In some embodiments, the method comprises subjecting a sample to a purification platform for purifying a target from a sample, wherein the purification platform comprises, in order: (a) a capture step, wherein the capture step comprises a protein A-based affinity medium comprising a rigid, high-flow agarose matrix and a protein A-derived ligand having alkaline stability, e.g., a MabSelect™ PrismA medium; (b) a MM-HIC/AEX chromatography step using a depth filtration step as a load filtration step, wherein the MM-HIC/AEX chromatography step comprises a medium comprising a multimodal strong anion exchanger using a N-benzyl-N-methyl ethanolamine ligand, e.g., a Capto™ Adhere medium, and wherein the depth filtration step comprises a depth filter comprising a silica, such as a silica filter aid, and a polyacrylic fiber, e.g., a X0SP depth filter, a C0SP depth filter, or a D0SP depth filter; and (c) a HIC step, wherein the HIC step is a flow-through mode HIC step, and wherein the HIC step comprises a medium comprising a cross-linked poly(styrene-divinylbenzene) POROS™-based bead with aromatic hydrophobic benzyl ligands, e.g., Poros™ Benzyl Ultra. In some embodiments, the HIC step comprises a pH adjustment step, wherein the pH of the input material is pH adjusted to a pH of about 4.5 to about 5.5, such as a about 5.0. In some embodiments, the HIC step is a low salt HIC step such as no salt, such as a HIC conditioning salt, is added prior to loading a material on the HIC membrane or the HIC column.

In some embodiments, the method comprises subjecting a sample to a purification platform for purifying a target from a sample, wherein the purification platform comprises, in order: (a) a capture step, wherein the capture step comprises a protein A-based affinity medium comprising a rigid, high-flow agarose matrix and alkali-stabilized protein A-derived ligand, wherein amino acids particularly sensitive to alkali were substituted with more stable residue in an alkali environment, e.g. a MabSelect SuRe™ medium; (b) a MM-HIC/AEX chromatography step using a depth filtration step as a load filtration step, wherein the MM-HIC/AEX chromatography step comprises a medium comprising a multimodal strong anion exchanger using a N-benzyl-N-methyl ethanolamine ligand, e.g., a Capto™ Adhere medium, and wherein the depth filtration step comprises a depth filter comprising a hydrogel Q (quaternary amine, also referred to as a quaternary ammonium)-functionalized non-woven material, and a multizone microporous membrane, e.g., EMPHAZE™ AEX depth filter; and (c) a HIC step, wherein the HIC step is a flow-through mode HIC step, and wherein the HIC step comprises a medium comprising a cross-linked 6% agarose bead modified with aromatic phenyl groups via an uncharged and chemically-stable ether linkage, wherein the medium comprises approximately 40-45 μmol phenyl/mL medium, e.g., a Phenyl SFF HS medium. In some embodiments, the HIC step comprises a pH adjustment step, wherein the pH of the input material is pH adjusted to a pH of about 5.0 to about 6.0, such as a about 5.5. In some embodiments, the HIC step is a low salt HIC step, such as no salt, such as a HIC conditioning salt, is added prior to loading a material on the HIC membrane or the HIC column.

In some embodiments, provided is a method comprising subjecting a sample to a purification platform for purifying a target from a sample, wherein the purification platform comprises: a capture step; one or more ion exchange (IEX) chromatography steps; and a hydrophobic interaction chromatography (HIC) step. In some embodiments, the IEX chromatography step is a CEX chromatography step. In some embodiments, the purification platform further comprises a virus filtration step. In some embodiments, the purification platform further comprises a UF/DF step. In some embodiments, the purification platform further comprises a depth filtration step. In some embodiments, the purification platform further comprises a HIC step. Exemplary purification platforms encompassed within the described purification platforms is shown in FIG. 1C and are described in more detail below.

In some embodiments, the method comprises subjecting a sample to a purification platform for purifying a target from a sample, wherein the purification platform comprises, in order: (a) a capture step; (b) a CEX chromatography step; (c) a HIC step; (d) a virus filtration step; and (e) a UF/DF step. In some embodiments, the capture step comprises a protein A-based affinity medium, e.g., a MabSelect SuRe™ or MabSelect™ PrimaA medium. In some embodiments, the capture step comprises a bind-and-elute mode affinity chromatography step. In some embodiments, the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., a Poros™ XS medium. In some embodiments, the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., Poros™ 50HS. In some embodiments, the HIC step comprises a medium comprising a cross-linked 6% agarose bead modified with aromatic phenyl groups via an uncharged and chemically-stable ether linkage, wherein the medium comprises approximately 40-45 μmol phenyl/mL medium, e.g., a Phenyl SFF HS medium. In some embodiments, the HIC step comprises a medium comprising a 100 nm pore size polymethacrylate-based material bonded with C6 groups (hexyl), e.g., a Toyopearl® Hexyl-650 medium. In some embodiments, the HIC step comprises a medium comprising a cross-linked poly(styrene-divinylbenzene) POROS™-based bead with aromatic hydrophobic benzyl ligands, e.g., Poros™ Benzyl Ultra.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a CEX chromatography step, wherein the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., a Poros™ XS medium; (c) a HIC step, wherein the HIC step comprises a medium comprising a cross-linked 6% agarose bead modified with aromatic phenyl groups via an uncharged and chemically-stable ether linkage, wherein the medium comprises approximately 40-45 μmol phenyl/mL medium, e.g., a Phenyl SFF HS medium; (d) a virus filtration step; and (e) a UF/DF step.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a CEX chromatography step, wherein the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., a Poros™ XS medium; (c) a HIC step, wherein the HIC step comprises a medium comprising a 100 nm pore size polymethacrylate-based material bonded with C6 groups (hexyl), e.g., a Toyopearl® Hexyl-650 medium; (d) a virus filtration step; and (e) a UF/DF step.

In some embodiments, the method comprises subjecting a sample to a purification platform for purifying a target from a sample, wherein the purification platform comprises, in order: (a) a capture step, wherein the capture step comprises a bind-and-elute mode affinity chromatography step; (b) a CEX chromatography step, wherein the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., a Poros™ XS medium; (c) a HIC step, wherein the HIC step comprises a medium comprising a cross-linked poly(styrene-divinylbenzene) POROS™-based bead with aromatic hydrophobic benzyl ligands, e.g., a Poros™ Benzyl Ultra medium; (d) a virus filtration step; and (e) a UF/DF step.

In some embodiments, the method comprises subjecting a sample to a purification platform for purifying a target from a sample, wherein the purification platform comprises, in order: (a) a capture step, wherein the capture step comprises a bind-and-elute mode affinity chromatography step; (b) a CEX chromatography step, wherein the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., a Poros™ 50HS medium; (c) a HIC step, wherein the HIC step comprises a medium comprising a cross-linked 6% agarose bead modified with aromatic phenyl groups via an uncharged and chemically-stable ether linkage, wherein the medium comprises approximately 40-45 μmol phenyl/mL medium, e.g., a Phenyl SFF HS medium; (d) a virus filtration step; and (e) a UF/DF step.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a CEX chromatography step, wherein the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., a Poros™ 50HS medium; (c) a HIC step, wherein the HIC step comprises a medium comprising a 100 nm pore size polymethacrylate-based material bonded with C6 groups (hexyl), e.g., a Toyopearl® Hexyl-650 medium; (d) a virus filtration step; and (e) a UF/DF step.

In some embodiments, the method comprises subjecting a sample to a purification platform for purifying a target from a sample, wherein the purification platform comprises, in order: (a) a capture step, wherein the capture step comprises a protein A-based affinity medium, e.g., a MabSelect SuRe™ or MabSelect™ PrimaA medium, and wherein the capture step is configured to be a bind-and-elute affinity chromatography step; (b) a CEX chromatography step, wherein the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., a Poros™ 50HS medium; (c) a HIC step, wherein the HIC step comprises a medium comprising a cross-linked poly(styrene-divinylbenzene) POROS™-based bead with aromatic hydrophobic benzyl ligands, e.g., a Poros™ Benzyl Ultra medium; (d) a virus filtration step; and (e) a UF/DF step. In some embodiments, the purification platform further comprises a depth filtration step, positioned at any position

In some embodiments, provided is a method comprising subjecting a sample to a purification platform for purifying a target from a sample, wherein the purification platform comprises: one or more ion exchange (IEX) chromatography steps; a hydrophobic interaction chromatography (HIC) step; and a depth filtration step. In some embodiments, the purification platform further comprises a virus filtration step and/or a UF/DF step. In some embodiments, the depth filtration step and/or the HIC step is directly before the virus filtration step of the UF/DF step. In some embodiments, the depth filtration step and/or the HIC step is sequential with or directly after the IEX chromatography step. Exemplary purification platforms encompassed within the described purification platforms is shown in FIG. 1D and described in more detail below.

In some embodiments, the method comprises subjecting a sample to a purification platform for purifying a target from a sample, wherein the purification platform comprises, in order: (a) a CEX chromatography step; (b) a HIC step; (c) a MMIEX chromatography step; (d) an AEX chromatography step; (e) a depth filter step; and (f) a UF/DF step. In some embodiments, the CEX chromatography step comprises a CEX chromatography medium comprising cross-linked 6% agarose beads having dextran chains covalently coupled to the agarose matrix that are modified with sulphopropyl (SP) strong cation exchange groups, e.g., a SP Sepharose® XL (SPXL) medium or a Streamline™ SPXL medium. In some embodiments, the HIC step comprises a HIC medium comprising propyl groups covalently linked to nitrogens on polyethylenimine (PEI) ligands attached to a substrate, e.g., a Bakerbond WP HI-Propyl™ medium. In some embodiments, the MMIEX chromatography step comprises a MMIEX chromatography medium comprising silica gel solid phase particles comprising a mixed mode anion/cation exchanger, e.g., a Bakerbond ABx™ medium. In some embodiments, the AEX chromatography step comprises an AEX chromatography medium comprising a cross-linked 6% agarose bead having quaternary ammonium (Q) strong anion exchange groups, e.g., a Q Sepharose® Fast Flow (QSFF) medium. In some embodiments, the depth filtration step is comprises a depth filter comprising a silica, such as a silica filter aid, and a polyacrylic fiber, e.g., a X0SP depth filter, a C0SP depth filter, or a D0SP depth filter.

In some embodiments, the method comprises subjecting a sample to a purification platform for purifying a target from a sample, wherein the purification platform comprises, in order: (a) a CEX chromatography step, wherein the CEX chromatography step comprises a CEX chromatography medium comprising cross-linked 6% agarose beads having dextran chains covalently coupled to the agarose matrix that are modified with sulphopropyl (SP) strong cation exchange groups, e.g., a SP Sepharose® XL (SPXL) medium or a Streamline™ SPXL medium; (b) a HIC step, wherein the HIC step comprises a HIC medium comprising propyl groups covalently linked to nitrogens on polyethylenimine (PEI) ligands attached to a substrate, e.g., a Bakerbond WP HI-Propyl™ medium; (c) a MMIEX chromatography step, wherein the MMIEX chromatography step comprises a MMIEX chromatography medium comprising silica gel solid phase particles comprising a mixed mode anion/cation exchanger, e.g., a Bakerbond ABx™ medium; (d) a AEX chromatography step, wherein the AEX chromatography step comprises an AEX chromatography medium comprising a cross-linked 6% agarose bead having quaternary ammonium (Q) strong anion exchange groups, e.g., a Q Sepharose® Fast Flow (QSFF) medium; (e) a depth filter step, wherein the depth filtration step is comprises a depth filter comprising a silica, such as a silica filter aid, and a polyacrylic fiber, e.g., a X0SP depth filter, a C0SP depth filter, or a D0SP depth filter; and (f) a UF/DF step. In some embodiments, the purification platform further comprises a depth filtration step, positioned at any position, wherein the depth filtration step comprises a depth filter comprising a silica, such as a silica filter aid, and a polyacrylic fiber, e.g., a X0SP depth filter, a C0SP depth filter, or a D0SP depth filter, and wherein the depth filtration step is configured to be performed at, e.g., the input material has, a pH of about 6 to about 7, such as about 6.5. In some embodiments, the purification platform further comprises a depth filtration step, positioned at any position, wherein the depth filtration step comprises a depth filter comprising a hydrogel Q (quaternary amine, also referred to as a quaternary ammonium)-functionalized non-woven material, and a multizone microporous membrane, e.g., an EMPHAZE™ AEX depth filter, and wherein the depth filtration step is configured to be performed at, e.g., the input material has, a pH of about 8.5 to about 9.5, such as about 9.1. In some embodiments, the purification platform further comprises a HIC step, positioned at any position, wherein the HIC step comprises a HIC medium, such as a HIC membrane, comprising a phenyl moiety conjugated to a stabilized reinforced cellulose filter, e.g., a Sartobind® Phenyl medium, wherein the HIC step is configured to be performed at, e.g., the input material has, a pH of about 6 to about 7, such as about 6.5.

In some embodiments, provided is a method comprising subjecting a sample to a purification platform for purifying a target from a sample, wherein the purification platform comprises: (a) a capture step; (b) one or more ion exchange (IEX) chromatography steps, such as a CEX chromatography step; (c) a multimodal hydrophobic interaction/ion exchange (MM-HIC/IEX) chromatography steps; and (d) one or both of: (i) a hydrophobic interaction chromatography (HIC) step; and (ii) a depth filtration step. In some embodiments, the purification platform further comprises a virus filtration step. In some embodiments, the purification platform further comprises a UF/DF step. Exemplary purification platforms encompassed within the described purification platforms is shown in FIG. 1E. In some embodiments, the capture step comprises a protein A-based affinity medium, e.g., a MabSelect SuRe™ or MabSelect™ PrimaA medium. In some embodiments, the capture step comprises a bind-and-elute mode affinity chromatography step. In some embodiments, the CEX chromatography step comprises a CEX chromatography medium comprising rigid polymeric resin particles comprising cross-linked poly[styrene-divinylbenzene]having a polyhydroxyl surface coating further functionalization with sulphopropyl (SP) strong cation exchange groups, e.g., a Poros™ XS medium. In some embodiments, the CEX chromatography step comprises a CEX chromatography medium comprising rigid polymeric resin particles comprising cross-linked poly[styrene-divinylbenzene] having a polyhydroxyl surface coating further functionalization with sulphopropyl (SP) strong cation exchange groups, e.g., Poros™ 50HS. In some embodiments, the MM-HIC/AEX chromatography step comprises a medium comprising a multimodal strong anion exchanger using a N-benzyl-N-methyl ethanolamine ligand, e.g., a Capto™ Adhere medium. In some embodiments, the purification platform further comprises a depth filtration step, positioned at any position.

### III. Additional Steps and Methods

In some aspects, the present disclosure provides additional steps involved or associated with a purification platform described herein. Additional steps involved or associated with a purification platform, and methods for conducting such steps, are known. See, e.g., Liu et al., mAbs, 2, 2010, which is hereby incorporated herein by reference in its entirety.

In some embodiments, the method further comprises a cell culture step. In some embodiments, the method further comprises a sample processing step, such as a sample preparation step. In some embodiments, the method further comprises a clarification step, such as to clarify HCCF. In some embodiments, the method further comprises a host cell and host cell debris removal step, such as to remove host cells and host cell debris from a sample and/or a composition obtained from the purification platform. In some embodiments, the method further comprises a centrifugation step. In some embodiments, the method further comprises a sterile filtration step. In some embodiments, the method further comprises a tangential flow micro-filtration step. In some embodiments, the method further comprises a flocculation/precipitation step.

In some embodiments, the method further comprises a formulation step, such as processing a composition to form a pharmaceutically acceptable composition, or a precursor thereof.

In some embodiments, the method further comprises determining the hydrolytic enzyme activity rate of the composition. In some embodiments, the method further comprises performing a hydrolytic activity assay on a composition obtained from a purification platform described herein. In some embodiments, the hydrolytic activity assay comprises measuring the hydrolytic activity of one or more hydrolytic enzymes by monitoring the conversion of a substrate, such as a non-fluorescent substrate, to a detectable product of the hydrolytic enzyme, such as a fluorescent product. In some embodiments, the substrate comprises an ester bond. In some embodiments, the method further comprises determining the product of one or more hydrolytic enzymes, e.g., as described in WO2018035025, which is hereby incorporated by reference in its entirety. In some embodiments, the method further comprises determining the level of free fatty acids (FFA) in a composition obtained from a purification platform described herein by performing a Fatty Acid Mass Spectrometry (FAMS) assay. In some embodiments, to monitor the content of free fatty acids after PS20 degradation in the respective elution pools, samples were first prepared for PS20 stability studies and subsequently analyzed by mass spectrometry. In some embodiments, the method further comprises determining the level of one or more hydrolytic enzymes in the composition. In some embodiments, the method further comprises a determining a shelf-life of a composition. In some embodiments, the method further comprises determining the level of aggregates of a target in a composition.

In some embodiments, the hydrolytic activity assay (LEAP assay) comprises measuring the hydrolytic activity by monitoring the conversion of a non-fluorescent substrate to a fluorescent product through the cleavage of the substrate ester bond. In some embodiments, the hydrolytic activity in [μM MU/h] is determined by subtracting the reaction rate of the enzyme blank (k_{self}-cleavage [RFU/h]) from the reaction rate of the sample (k_{raw} [RFU/h]), and converting the fluorescent signal to μM MU/h by dividing the term by the conversion factor a [RFU/μM]. In some embodiments, the hydrolytic activities are normalized to the protein concentration applied per well. In some embodiments, the hydrolytic activity is reported as a percent of the hydrolytic activity of the reference sample when the reference sample represents 100% hydrolytic activity.

In some embodiments, the Fatty Acid Mass Spectrometry (FAMS) assay comprises obtaining extracted ion chromatograms (XICs) for the masses of lauric acid, myristic acid, and isotopically labelled (D₂₃)-lauric acid and (¹³C₁₄) myristic acid. In some embodiments, the respective peaks are integrated and the peak area ratio between lauric acid and D₂₃-lauric acid as well as the ratio between myristic acid and ¹³C₁₄-myristic acid are determined. In some embodiments, the peak area ratio is used to calculate the concentrations of lauric acid and/or myristic acid in the samples. In some embodiments, the amount FFA (lauric acid (LA) and/or myristic acid (MA)) is reported as a percent when the amount of the reference sample is set to 100%.

### IV. Samples and Components Thereof

In some embodiments, the purification platforms and methods described herein are useful for purifying, to any degree, a target from a sample comprising the target.

In some embodiments, the sample is a host cell sample. In some embodiments, the sample is a host cell culture fluid (HCCF). In some embodiments, the sample comprises a portion of a host cell culture fluid. In some embodiments, the sample is derived from a host cell culture fluid. In some embodiments, the sample comprises a host cell. In some embodiments, the sample comprises a component of a host cell, such as host cell debris. In some embodiments, the host cell is a bacterial cell. In some embodiments, the host cell is an E. coli cell. In some embodiments, the host cell is an insect cell. In some embodiments, the host cell is a mammalian cell. In some embodiments, the host cell is a Chinese hamster ovary (CHO) cell. In some embodiments, the host cell is a human cell.

In some embodiments, the sample has been processed, such as subjected to a processing step performed prior to subjecting the sample to a purification platform described herein. In some embodiments, the sample comprises a surfactant. In some embodiments, the sample comprises a polysorbate. In some embodiments, the polysorbate is selected from the group consisting of polysorbate 20, polysorbate 40, polysorbate 60, and polysorbate 80.

In some embodiments, the sample comprises a target. In some embodiments, the target comprises a polypeptide. In some embodiments, the target is a polypeptide. In some embodiments, the target is a recombinant polypeptide. In some embodiments, the target is a polypeptide complex. In some embodiments, the target is an antibody moiety. In some embodiments, the antibody moiety is a monoclonal antibody. In some embodiments, the antibody moiety is a humanized antibody. In some embodiments, the antibody moiety is selected from the group consisting of an anti-TAU antibody, an anti-TGFβ3 antibody, an anti-VEGF-A antibody, an anti-CD20 antibody, an anti-CD40 antibody, an anti-HER2 antibody, an anti-IL6 antibody, an anti-IgE antibody, an anti-IL13 antibody, an anti-TIGIT antibody, an anti-PD-L1 antibody, an anti-VEGF-A/ANG2 antibody, an anti-CD79b antibody, an anti-ST2 antibody, an anti-factor D antibody, an anti-factor IX antibody, an anti-factor X antibody, an anti-abeta antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, a FAP-IL2v fusion protein, and a TYRP1 TCB antibody. In some embodiments, the antibody moiety is selected from the group consisting of ocrelizumab, pertuzumab, ranibizumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, omalizumab ranibizumab, emicizumab, selicrelumab, prasinezumab, R06874281, and R07122290.

In some embodiments, the sample comprises one or more host cell proteins. In some embodiments, the host cell protein is a hydrolytic enzyme. In some embodiments, the hydrolytic enzyme is a lipase, an esterase, a thioesterase, a phospholipase, carboxylesterase, hydrolase, cutinase, or a ceramidase. In some embodiments, the hydrolytic enzyme is a multi-enzyme protein. In some embodiments, the multi-enzyme protein is a fatty acid synthase. In some embodiments, the fatty acid synthase comprises a thioesterase subunit.

In some embodiments, the sample has a baseline hydrolytic enzyme activity rate, such as measured by a hydrolytic enzyme activity assay described herein. In some embodiments, the baseline hydrolytic enzyme activity rate of the sample is based on, at least in part, the presence of a host cell hydrolytic enzyme. In some embodiments, the hydrolytic enzyme is a lipase, an esterase, a thioesterase, a phospholipase, carboxylesterase, hydrolase, cutinase, or a ceramidase.

In some embodiments, the sample comprises an added component, such as a surfactant, e.g., a polysorbate. In some embodiments, the component is added to the sample prior to purification, such as to a HCCF.

### V. Compositions Obtained from the Purification Platforms and Pharmaceutical Compositions

The purification platforms described herein comprise numerous steps. In some embodiments, the term “composition” is used herein to describe any input (except the initial sample input to the purification platform), intermediate, or output of any stage of the purification platform. For example, in some embodiments, use of the term “composition” is not limited to describing the final output of the purification platform. In some embodiments, the composition has been processed by a HIC step and/or a depth filtration step and/or a MM-HIC/IEX chromatography step.

In some embodiments, the composition comprises a surfactant. In some embodiments, the composition comprises a polysorbate. In some embodiments, the polysorbate is selected from the group consisting of polysorbate 20, polysorbate 40, polysorbate 60, and polysorbate 80.

In some embodiments, the composition comprises a target. In some embodiments, the target comprises a polypeptide. In some embodiments, the target is a polypeptide. In some embodiments, the target is a polypeptide complex. In some embodiments, the target is an antibody moiety. In some embodiments, the antibody moiety is a monoclonal antibody. In some embodiments, the antibody moiety is a humanized antibody. In some embodiments, the antibody moiety is selected from the group consisting of an anti-TAU antibody, an anti-TGFβ3 antibody, an anti-VEGF-A antibody, an anti-CD20 antibody, an anti-CD40 antibody, an anti-HER2 antibody, an anti-IL6 antibody, an anti-IgE antibody, an anti-IL13 antibody, an anti-TIGIT antibody, an anti-PD-L1 antibody, an anti-VEGF-A/ANG2 antibody, an anti-CD79b antibody, an anti-ST2 antibody, an anti-factor D antibody, an anti-factor IX antibody, an anti-factor X antibody, an anti-abeta antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, a FAP-IL2v fusion protein, and a TYRP1 TCB antibody. In some embodiments, the antibody moiety is selected from the group consisting of ocrelizumab, pertuzumab, ranibizumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, omalizumab ranibizumab, emicizumab, selicrelumab, prasinezumab, R06874281, and R07122290.

In some embodiments, the composition comprises one or more host cell proteins. In some embodiments, the host cell protein is a hydrolytic enzyme. In some embodiments, the hydrolytic enzyme is a lipase, an esterase, a thioesterase, a phospholipase, carboxylesterase, hydrolase, cutinase, or a ceramidase.

In some embodiments, the composition has a reduced hydrolytic enzyme activity rate, wherein the reduction in the hydrolytic enzyme activity rate is at least about 20%, such as at least about any of 25%, 30%, 35%, 40%, 45%, 50%, 55%, 60%, 65%, 70%, 75%, 80%, 85%, 90%, or 95%, as compared to a relevant references, such as a composition obtained from a purification platform not comprising a HIC step and/or a depth filtration step and/or a MM-HIC/IEX chromatography step. In some embodiments, the reference is from the sample purified using the same purification platform without one or more of the depth filtration steps and/or the one or more of the HIC steps and/or a MM-HIC/IEX chromatography step.

In some embodiments, the present disclosure provides pharmaceutical compositions obtained from the purification platforms described herein. In some embodiments, the pharmaceutical composition is obtained from a method described herein. In some embodiments, the pharmaceutical composition is a purified composition. In some embodiments, the pharmaceutical composition is a sterile pharmaceutical composition.

In some embodiments, the pharmaceutical composition comprises an antibody moiety. In some embodiments, the pharmaceutical composition comprises an antibody moiety and a polysorbate. In some embodiments, the pharmaceutical composition comprises an antibody moiety, a polysorbate, and a host cell impurity, such as a host cell protein, e.g., a hydrolytic enzyme.

In some embodiments, the pharmaceutical composition comprises a polysorbate. In some embodiments, the pharmaceutical composition is selected from the group consisting of polysorbate 20, polysorbate 40, polysorbate 60, and polysorbate 80.

In some embodiments, the pharmaceutical composition has a reduced hydrolytic enzyme activity rate, wherein the reduction in the hydrolytic enzyme activity rate is at least about 20%, such as at least about any of 25%, 30%, 35%, 40%, 45%, 50%, 55%, 60%, 65%, 70%, 75%, 80%, 85%, 90%, or 95%, as compared to a relevant references, such as a pharmaceutical composition obtained from a purification platform not comprising a HIC step and/or a depth filtration step and/or a MM-HIC/IEX chromatography step. In some embodiments, the reference is from the sample purified using the same purification platform without one or more of the depth filtration steps and/or the one or more of the HIC steps and/or the one or more MM-HIC/IEX chromatography steps.

In some embodiments, the pharmaceutical composition has a reduced level of one or more hydrolytic enzymes, as compared to a composition obtained from purification of the same sample using the same purification platform without one or more of the depth filtration steps and/or one or more of the HIC steps and/or one or more MM-HIC/IEX chromatography steps.

In some embodiments, the pharmaceutical composition has reduced degradation of a polysorbate, as compared to a composition obtained from purification of the same sample using the same purification platform without one or more of the depth filtration steps and/or one or more of the HIC steps and/or one or more MM-HIC/IEX chromatography steps.

In some embodiments, the pharmaceutical composition has increased shelf-life, as compared to a composition obtained from purification of the same sample using the same purification platform without one or more of the depth filtration steps and/or one or more of the HIC steps and/or one or more MM-HIC/IEX chromatography steps.

In some embodiments, the pharmaceutical composition has less degraded polysorbate, as compared to a composition obtained from purification of the same sample using the same purification platform without one or more of the depth filtration steps and/or one or more of the HIC steps and/or one or more MM-HIC/IEX chromatography steps.

In some embodiments, the pharmaceutical composition has reduced aggregation of a target, as compared to a composition obtained from purification of the same sample using the same purification platform without one or more of the depth filtration steps and/or one or more of the HIC steps and/or one or more MM-HIC/IEX chromatography steps.

In some aspects, the present disclosure provides formulated antibody moiety compositions obtained from the purification platforms described herein. In some embodiments, the formulated antibody moiety composition is obtained from a method described herein.

In some embodiments, the formulated antibody moiety composition comprises an antibody moiety. In some embodiments, the formulated antibody moiety composition comprises an antibody moiety and a polysorbate. In some embodiments, the formulated antibody moiety composition comprises an antibody moiety, a polysorbate, and a host cell impurity, such as a host cell protein, e.g., one or more hydrolytic enzymes.

In some embodiments, the formulated antibody moiety compositions described herein have an increased shelf-life as compared to a reference, such as a formulated antibody moiety composition obtained from the same purification platform without one or more of the depth filtration steps and/or one or more of the HIC steps and/or one or more MM-HIC/IEX chromatography steps. In some embodiments, the shelf-life is assessed, such as measured, via aggregation of an antibody moiety of a formulated antibody moiety composition. In some embodiments, the shelf-life is assessed, such as measured, via preservation of one or more functionalities of an antibody moiety of a formulated antibody moiety composition. In some embodiments, the shelf-life is assessed, such as measured, via activity, such as binding activity, of an antibody moiety of a formulated antibody moiety composition.

In some embodiments, the formulated antibody moiety composition comprising an antibody moiety and a polysorbate has a reduced rate of polysorbate hydrolysis, wherein the shelf-life of the composition is more than about 8 months, such as more than about any of 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months. In some embodiments, the formulated antibody moiety composition having a reduced rate of polysorbate hydrolysis is as compared to a reference, such as a formulated antibody moiety composition obtained from the same purification platform without one or more of the depth filtration steps and/or one or more of the HIC steps and/or one or more MM-HIC/IEX chromatography steps. In some embodiments, the reduced rate of polysorbate hydrolysis is a reduced relative rate of polysorbate hydrolysis.

In some embodiments, the formulated antibody moiety composition comprising an antibody moiety and a polysorbate has a reduced rate of polysorbate hydrolysis, wherein the shelf-life of the composition is extended compared to the shelf-life indicated in documents filed with a health authority related to the formulated antibody moiety composition, and wherein the shelf-life is extended by at least about 2 months, such as at least about any of 3 months, 4 months, 5 months, 6 months, 7 months, 8 months, 9 months, 10 months, 11 months, or 12 months, as compared to the shelf-life indicated in said documents. In some embodiments, the formulated antibody moiety composition having a reduced rate of polysorbate hydrolysis is as compared to a reference, such as a formulated antibody moiety composition obtained from the same purification platform without one or more of the depth filtration steps and/or one or more of the HIC steps and/or the one or more MM-HIC/IEX chromatography steps. In some embodiments, the reduced rate of polysorbate hydrolysis is a reduced relative rate of polysorbate hydrolysis.

In some embodiments, the formulated antibody moiety composition comprising an antibody moiety and a polysorbate has a reduced degradation of polysorbate, wherein the degradation is reduced by at least about 5%, such as at least about any of 10%, 15%, 20%, 25%, 30%, 35%, 40%, 45%, 50%, 55%, 60%, 65%, 70%, 75%, 80%, 85%, 90%, 95%, or 100%, as compared to the degradation indicated in documents filed with a health authority related to the formulated antibody moiety composition. In some embodiments, the formulated antibody moiety composition having a reduced degradation of polysorbate is as compared to a reference, such as a formulated antibody moiety composition obtained from a same purification platform without one or more of the depth filtration steps and/or one or more of the HIC steps and/or a MM-HIC/IEX chromatography step. In some embodiments, the reduced degradation of polysorbate is a reduced relative degradation of polysorbate.

In some embodiments, the rate of polysorbate hydrolysis of a formulated antibody moiety composition is reduced by at least about 5%, such as at least about any of 10%, 15%, 20%, 25%, 30%, 35%, 40%, 45%, 50%, 55%, 60%, 65%, 70%, 75%, 80%, 85%, 90%, 95%, or 100%, as compared to a reference.

In some embodiments, the formulated antibody moiety composition comprises an antibody moiety and a polysorbate, wherein the polysorbate is degraded during storage of the liquid composition by about 60% or less per year, such as about any of 55% or less per year, 50% or less per year, 45% or less per year, 40% or less per year, 35% or less per year, 30% or less per year, 25% or less per year, 20% or less per year, 15% or less per year, 10% or less per year, or 5% or less per year.

In some embodiments, the formulated antibody moiety compositions described herein have reduced aggregate formation for at least about 6 months, such as at least about any of 7 months, 8 months, 9 months, 10 months, 11 months, 12 months, 13 months, 14 months, 15 months, 16 months, 17 months, 18 months, 19 months, 20 months, 21 months, 22 months, 23 months, or 24 months, as compared to a reference, such as a formulated antibody moiety composition obtained from the same purification platform without one or more of the depth filtration steps and/or one or more of the HIC steps and/or one or more MM-HIC/IEX chromatography steps. In some embodiments, the formulated antibody moiety compositions described herein have at least about 20% less, such as at least about any of 25% less, 30% less, 35% less, 40% less, 45% less, 50% less, 55% less, 65% less, 70% less, 75% less, 80% less, 85% less, 90% less, 95% less, or 100% less, aggregate formation as compared to a reference for at least about 6 months, such as at least about any of 7 months, 8 months, 9 months, 10 months, 11 months, 12 months, 13 months, 14 months, 15 months, 16 months, 17 months, 18 months, 19 months, 20 months, 21 months, 22 months, 23 months, or 24 months, wherein the reference is a formulated antibody moiety composition obtained from the same purification platform without one or more of the depth filtration steps and/or one or more of the HIC steps and/or one or more MM-HIC/IEX chromatography steps. Methods for assessing, such as measuring, aggregate formation are known in the art and include, e.g., visual inspection, dynamic light scattering, static light scattering, and optical density measurements.

In some embodiments, the formulated antibody moiety compositions described herein maintain at least about 50%, such as at least about any of 55%, 60%, 70%, 75%, 80%, 85%, 90%, 95%, 96%, 97%, 98%, 99%, of the antibody moiety activity, such as compared to a reference for at least about 6 months, such as at least about any of 7 months, 8 months, 9 months, 10 months, 11 months, 12 months, 13 months, 14 months, 15 months, 16 months, 17 months, 18 months, 19 months, 20 months, 21 months, 22 months, 23 months, or 24 months, wherein the reference is a formulated antibody moiety composition obtained from the same purification platform without one or more of the depth filtration steps and/or one or more of the HIC steps and/or one or more MM-HIC/IEX chromatography steps.

In some embodiments, the antibody moiety is a monoclonal antibody.

In some embodiments, the antibody moiety is a human, humanized, or chimeric antibody.

In some embodiments, the antibody is selected from the group consisting of an anti-TAU antibody, an anti-TGFβ3 antibody, an anti-VEGF-A antibody, an anti-CD20 antibody, an anti-CD40 antibody, an anti-HER2 antibody, an anti-IL6 antibody, an anti-IgE antibody, an anti-IL13 antibody, an anti-TIGIT antibody, an anti-PD-L1 antibody, an anti-VEGF-A/ANG2 antibody, an anti-CD79b antibody, an anti-ST2 antibody, an anti-factor D antibody, an anti-factor IX antibody, an anti-factor X antibody, an anti-abeta antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, a FAP-IL2v fusion protein, and a TYRP1 TCB antibody.

In some embodiments, the antibody moiety is selected from the group consisting of ocrelizumab, pertuzumab, ranibizumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, omalizumab ranibizumab, emicizumab, selicrelumab, prasinezumab, RO6874281, and RO7122290.

In some embodiments, the polysorbate is selected from the group consisting of polysorbate 20, polysorbate 40, polysorbate 60, and polysorbate 80.

Further aspects reported herein are formulated antibody compositions with low polysorbate degradation during storage. One aspect of the invention is a formulated antibody composition comprising an antibody/protein and a polysorbate, wherein the polysorbate is degraded during storage/shelf life of the formulated antibody composition by about 50% or less (such as about any of 45% or less, 40% or less, 35% or less, 30% or less, 25% or less, 20% or less, 15% or less, 10% or less, 9% or less, 8% or less, 7% or less, 6% or less, 5% or less, 4% or less, 3% or less, 2% or less, or 1% or less, per year. In one embodiment the polysorbate is degraded during storage of the liquid composition by 10% or less per year.

Another aspect is a formulated antibody composition comprising an antibody and a polysorbate, wherein after one year the polysorbate is present in the composition at a concentration of at least about 50%, such as at least about any of 55%, 60%, 65%, 70%, 75%, 80%, 85%, 90%, 91%, 92%, 93%, 94%, 95%, 96%, 97%, 98%, or 99%, of the initial concentration, wherein the initial concentration is the concentration upon formulation or beginning of storage of the antibody in the liquid composition. Those skilled in the art will recognize that several embodiments are possible within the scope and spirit of the disclosure of this application. The disclosure is illustrated further by the examples below, which are not to be construed as limiting the disclosure in scope or spirit to the specific procedures described therein.

### EXEMPLARY EMBODIMENTS

Embodiment 1. A method of reducing a hydrolytic enzyme activity rate of a composition obtained from a purification platform, the method comprising subjecting a sample to the purification platform comprising: a capture step; one or more ion exchange (IEX) chromatography steps; and a depth filtration step, thereby reducing the hydrolytic enzyme activity rate of the composition as compared to purification of the sample without the depth filtration step.

Embodiment 2. The method of embodiment 1, wherein each of the one or more IEX chromatography steps is selected from the group consisting of: an anion exchange (AEX) chromatography step, a cation exchange (CEX) chromatography step, and a multimodal ion exchange (MMIEX) chromatography step.

Embodiment 3. The method of embodiment 2, wherein the MMIEX chromatography step comprises a multimodal cation exchange/anion exchange (MM-AEX/CEX) chromatography step.

Embodiment 4. The method of any one of embodiments 1-3, further comprising a virus filtration step.

Embodiment 5. The method of any one of embodiments 1-4, further comprising an ultrafiltration/diafiltration (UF/DF) step.

Embodiment 6. The method of embodiment 5, wherein the purification platform comprises, in order: the capture step; the CEX chromatography step; the AEX chromatography step; the depth filtration step; the virus filtration step; and the UF/DF step.

Embodiment 7. The method of any one of embodiments 1-6, wherein the depth filtration step comprises processing via a depth filter, and wherein the depth filter is a X0SP depth filter, a C0SP depth filter, a D0SP depth filter, a Polisher ST depth filter, or an EMPHAZE™ depth filter.

Embodiment 8. The method of any one of embodiments 1-6, further comprising a HIC step comprising processing via Sartobind® phenyl.

Embodiment 9. A method of reducing a hydrolytic enzyme activity rate of a composition obtained from a purification platform, the method comprising subjecting a sample to the purification platform comprising: a capture step; a multimodal hydrophobic interaction/ion exchange (MM-HIC/IEX) chromatography step; and a hydrophobic interaction chromatography (HIC) step, thereby reducing the hydrolytic enzyme activity rate of the composition as compared to purification of the sample without the HIC step and/or the MM-HIC/IEX chromatography step.

Embodiment 10. The method of embodiment 9, wherein the MM-HIC/IEX chromatography step comprises processing via a MM-HIC/IEX chromatography medium and the processing is performed at a pH of about 4.5 to about 9.

Embodiment 11. The method of embodiment 9 or 10, wherein the MM-HIC/IEX chromatography step is a multimodal hydrophobic interaction/anion exchange (MM-HIC/AEX) chromatography step.

Embodiment 12. The method of embodiment 11, wherein the MM-HIC/AEX chromatography step comprises processing via Capto™ Adhere or Capto™ Adhere ImpRes.

Embodiment 13. The method of embodiment 9 or 10, wherein the MM-HIC/IEX chromatography step is a multimodal hydrophobic interaction/cation exchange (MM-HIC/CEX) chromatography step.

Embodiment 14. The method of embodiment 13, wherein the MM-HIC/CEX chromatography step comprises Capto™ MMC or Capto™ MMC ImpRes.

Embodiment 15. The method of any one of embodiments 9-14, wherein the purification platform comprises, in order: the capture step; the MM-HIC/IEX chromatography step; and the HIC step.

Embodiment 16. The method of any one of embodiments 9-15, further comprising a virus filtration step.

Embodiment 17. The method of any one of embodiments 9-16, further comprising an ultrafiltration/diafiltration (UF/DF) step.

Embodiment 18. The method of embodiment 17, wherein the purification platform comprises, in order: the capture step; the MM-HIC/AEX chromatography step; the HIC step; the virus filtration step; and the UF/DF step.

Embodiment 19. The method of any one of embodiments 9-18, further comprising a depth filtration step.

Embodiment 20. The method of embodiment 19, wherein the purification platform comprises, in order: the capture step; the depth filtration step; the MM-HIC/AEX chromatography step; and the HIC step.

Embodiment 21. The method of embodiment 19 or 20, wherein the depth filtration step comprises processing via a depth filter, and wherein the depth filter is a X0SP depth filter.

Embodiment 22. The method of embodiment 19 or 20, wherein the depth filtration step comprises processing via a depth filter, and the depth filter is an EMPHAZE™ depth filter or a Polisher ST depth filter.

Embodiment 23. The method of any one of embodiments 19-22, wherein the depth filter is used as a load filter in conjunction with the MM-HIC/AEX chromatography step.

Embodiment 24. The method of embodiment 23, wherein the MM-HIC/AEX chromatography step comprises processing via Capto™ Adhere or Capto™ Adhere ImpRes.

Embodiment 25. The method of embodiment 19, wherein the purification platform comprises, in order: the capture step; the MM-HIC/AEX chromatography step; the depth filtration step; and the HIC step.

Embodiment 26. The method of embodiment 25, wherein the depth filtration step comprises processing via a depth filter, and wherein the depth filter is a X0SP depth filter, a C0SP depth filter, or a D0SP depth filter.

Embodiment 27. The method of embodiment 25, wherein the depth filtration step comprises processing via a depth filter, and the depth filter is an EMPHAZE™ depth filter or a Polisher ST depth filter.

Embodiment 28. The method of embodiment any one of embodiments 25-27, wherein the MM-HIC/AEX chromatography step comprises processing via Capto™ Adhere or Capto™ Adhere ImpRes.

Embodiment 29. A method of reducing a hydrolytic enzyme activity rate of a composition obtained from a purification platform, the method comprising subjecting a sample to the purification platform comprising: a capture step; one or more ion exchange (IEX) chromatography steps; and a hydrophobic interaction chromatography (HIC) step, thereby reducing the hydrolytic enzyme activity rate of the composition as compared to purification of the sample without the HIC step.

Embodiment 30. The method of embodiment 29, wherein the one or more IEX chromatography steps is a cation exchange (CEX) chromatography step.

Embodiment 31. The method of embodiment 29 or 30, further comprising a virus filtration step.

Embodiment 32. The method of any one of embodiments 29-31, further comprising an ultrafiltration/diafiltration (UF/DF) step.

Embodiment 33. The method of embodiment 32, further comprising a depth filtration step performed at any stage prior to the UF/DF step.

Embodiment 34. The method of embodiment 33, wherein the purification platform comprises, in order: the capture step; the CEX chromatography step; the HIC step; the virus filtration step; and the UF/DF step.

Embodiment 35. A method of reducing a hydrolytic enzyme activity rate of a composition obtained from a purification platform, the method comprising subjecting a sample to the purification platform comprising: one or more ion exchange (IEX) chromatography steps; a hydrophobic interaction chromatography (HIC) step; and a depth filtration step, thereby reducing the hydrolytic enzyme activity rate of the composition as compared to purification of the sample without the HIC step or the depth filtration step.

Embodiment 36. The method of embodiment 35, wherein the reduction is as compared to purification of the sample without the HIC and the depth filtration step.

Embodiment 37. The method of embodiment 35 or 36, wherein each of the one or more IEX chromatography steps is selected from the group consisting of: an anion exchange (AEX) chromatography step, a cation exchange (CEX) chromatography step, and a multimodal ion exchange (MMIEX) chromatography step.

Embodiment 38. The method of embodiment 37, wherein the MMIEX chromatography step comprises a multimodal cation exchange/anion exchange (MM-AEX/CEX) chromatography step.

Embodiment 39. The method of any one of embodiments 35-38, further comprising an ultrafiltration/diafiltration (UF/DF) step.

Embodiment 40. The method of embodiment 39, wherein the purification platform comprises, in order: the CEX chromatography step; the HIC step; the MMIEX chromatography step; the AEX chromatography step; the depth filter step; and the UF/DF step.

Embodiment 41. A method of reducing a hydrolytic enzyme activity rate of a composition obtained from a purification platform, the method comprising subjecting a sample to the purification platform comprising: a capture step; one or more ion exchange (IEX) chromatography steps; a multimodal hydrophobic interaction/ion exchange (MM-HIC/IEX) chromatography steps; and one or both of: a hydrophobic interaction chromatography (HIC) step; and a depth filtration step, thereby reducing the hydrolytic enzyme activity rate of the composition as compared to purification of the sample without the HIC step or the depth filtration step.

Embodiment 42. The method of embodiment 41, wherein the reduction is as compared to purification of the sample without the HIC and the depth filtration step.

Embodiment 43. The method of embodiment 41 or 42, further comprising a virus filtration step.

Embodiment 44. The method of any one of embodiments 41-43, further comprising an ultrafiltration/diafiltration (UF/DF) step.

Embodiment 45. The method of any one of embodiments 41-44, wherein the depth filtration step is performed as a load filter for the MM-HIC/IEX chromatography step, as a load filter for the HIC step, or following the HIC step.

Embodiment 46. The method of any one of embodiment 41-45, wherein each of the one or more IEX chromatography steps is selected from the group consisting of: a cation exchange (CEX) chromatography step, an anion exchange (AEX) chromatography step, and a multimodal ion exchange (MMIEX) chromatography step.

Embodiment 47. The method of any one of embodiments 41-46, wherein the MM-HIC/IEX chromatography step is a multimodal hydrophobic interaction/anion exchange (MM-HIC/AEX) chromatography step.

Embodiment 48. The method of embodiment 47, wherein the MM-HIC/AEX chromatography step comprises processing via Capto™ Adhere or Capto™ Adhere ImpRes.

Embodiment 49. The method of any one of embodiments 1-34 and 41-48, wherein the capture step comprises processing via affinity chromatography.

Embodiment 50. The method of any one of embodiments 1-34 and 41-48, wherein the capture step is performed in a bind-and-elute mode.

Embodiment 51. The method of embodiment 49 or 50, wherein the affinity chromatography is selected from the group consisting of a protein A chromatography, a protein G chromatography, a protein A/G chromatography, a FcXL chromatography, a protein XL chromatography, a kappa chromatography, and a kappaXL chromatography.

Embodiment 52. The method of any one of embodiments 1-6, 19, 20, and 35-51, wherein the depth filtration step comprises processing via a depth filter.

Embodiment 53. The method of embodiment 52, wherein the depth filter is used as a load filter.

Embodiment 54. The method of embodiment 52 or 53, wherein the depth filter comprises a substrate comprising one or more of a diatomaceous earth composition, a silica composition, a cellulose fiber, a polymeric fiber, a cohesive resin, and an ash composition.

Embodiment 55. The method of embodiment 54, wherein at least a portion of the substrate of the depth filter comprises a surface modification.

Embodiment 56. The method of embodiment 55, wherein the surface modification is one or more of a quaternary amine surface modification, a cationic surface modification, and an anionic surface modification.

Embodiment 57. The method of any one of embodiments 52-56, wherein the depth filter is selected from the group consisting of: a X0SP depth filter, a D0SP depth filter, a C0SP depth filter, an EMPHAZE™ depth filter, a PDD1 depth filter, a PDE1 depth filter, a PDH5 depth filter, a ZETA PLUS™ 120ZA depth filter, a ZETA PLUS™ 120ZB depth filter, a ZETA PLUS™ DELI depth filter, a ZETA PLUS™ DELP depth filter, and a Polisher ST depth filter.

Embodiment 58. The method of embodiment 57, wherein the depth filter is the X0SP depth filter, the D0SP depth filter, or the C0SP depth filter, and wherein the processing via the depth filter is performed at a pH of about 4.5 to about 8.

Embodiment 59. The method of embodiment 57, wherein the depth filter is the EMPHAZE™ depth filter, and wherein processing via the depth filter is performed at a pH of about 7 to about 9.5.

Embodiment 60. The method of embodiment 57, wherein the depth filter is the Polisher ST depth filter, and wherein processing via the depth filter is performed at a pH of about 4.5 to about 9.

Embodiment 61. The method of any one of embodiments 9-60, wherein the HIC step comprises processing via a HIC membrane or a HIC column.

Embodiment 62. The method of embodiment 61, wherein processing via the HIC membrane or the HIC column is performed using low salt concentrations.

Embodiment 63. The method of any one of embodiments 59-61, wherein processing via the HIC membrane or the HIC column is performed in flow-through mode.

Embodiment 64. The method of any one of embodiments 61-63, wherein the HIC membrane or HIC column comprises a substrate comprising one or more of an ether group, an ethyl group, a propyl group, an isopropyl group, a butyl group, a hexyl group, an octyl group, and a phenyl group.

Embodiment 65. The method of any one of embodiments 61-64, wherein the HIC membrane or the HIC column is selected from the group consisting of Bakerbond WP HI-Propyl™ Phenyl Sepharose® Fast Flow (Phenyl-SFF), Phenyl Sepharose® Fast Flow Hi-sub (Phenyl-SFF HS), Toyopearl® Hexyl-650C, Toyopearl® Hexyl-650M, Toyopearl® Hexyl-650S, Poros™ Benzyl Ultra, and Sartobind® phenyl.

Embodiment 66. The method of embodiment 64 or 65, wherein processing via the HIC membrane or the HIC column is performed at a pH of about 4.5 to about 7.

Embodiment 67. The method of any one of embodiments 1-8 and 29-66, wherein each of the one or more IEX chromatography steps comprises processing via an IEX chromatography membrane or an IEX chromatography column.

Embodiment 68. The method of embodiment 67, wherein the IEX chromatography membrane or the IEX chromatography column is selected from the group consisting of: SPSFF, QSFF, SPXL, Streamline™ SPXL, ABx™, Poros™ XS, Poros™ 50HS, DEAE, DMAE, TMAE, QAE, and MEP-Hypercel™.

Embodiment 69. The method of any one of embodiments 1-68, wherein the purification platform is for purification of a target from the sample, and wherein the sample comprises the target and one or more host cell impurities.

Embodiment 70. The method of embodiment 69, wherein the target comprises a polypeptide.

Embodiment 71. The method of any one of embodiments 1-70, wherein the target is an antibody moiety.

Embodiment 72. The method of embodiment 71, wherein the antibody moiety is a monoclonal antibody.

Embodiment 73. The method of embodiment 71 or 72, wherein the antibody moiety is a human, humanized, or chimeric antibody.

Embodiment 74. The method of any one of embodiments 71-73, wherein the antibody moiety is selected from the group consisting of: an anti-TAU antibody, an anti-TGFβ3 antibody, an anti-VEGF-A antibody, an anti-CD20 antibody, an anti-CD40 antibody, an anti-HER2 antibody, an anti-IL6 antibody, an anti-IgE antibody, an anti-IL13 antibody, an anti-TIGIT antibody, an anti-PD-L1 antibody, an anti-VEGF-A/ANG2 antibody, an anti-CD79b antibody, an anti-ST2 antibody, an anti-factor D antibody, an anti-factor IX antibody, an anti-factor X antibody, an anti-abeta antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, a FAP-IL2v fusion protein, and a TYRP1 TCB antibody.

Embodiment 75. The method of any one of embodiments 71-74, wherein the antibody moiety is selected from the group consisting of: ocrelizumab, pertuzumab, ranibizumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, omalizumab, ranibizumab, emicizumab, selicrelumab, prasinezumab, R06874281, and R07122290.

Embodiment 76. The method of any one of embodiments 69-75, wherein the one or more host cell impurities comprises a host cell protein.

Embodiment 77. The method of embodiment 76, wherein the host cell protein is a hydrolytic enzyme.

Embodiment 78. The method of embodiment 77, wherein the hydrolytic enzyme is a lipase, an esterase, a thioesterase, a phospholipase, carboxylesterase, hydrolase, cutinase, or a ceramidase.

Embodiment 79. The method of any one of embodiments 1-78, wherein the sample comprises a host cell or components originating therefrom.

Embodiment 80. The method of any one of embodiments 1-79, wherein the sample is, or is derived from, a cell culture sample.

Embodiment 81. The method of embodiment 80, wherein the cell culture sample comprises a host cell, and wherein the host cell is a Chinese hamster ovary (CHO) cell or an E. coli cell.

Embodiment 82. The method of any one of embodiments 1-81, further comprising a sample processing step.

Embodiment 83. The method of any one of embodiments 1-82, wherein the reduction in the hydrolytic enzyme activity rate is at least about 20%.

Embodiment 84. The method of any one of embodiments 1-83, further comprising determining the hydrolytic enzyme activity rate of the composition.

Embodiment 85. The method of any one of embodiments 1-84, further comprising determining the level of one or more hydrolytic enzymes in the composition.

Embodiment 86. The method of any one of embodiments 1-85, wherein the composition comprises a polysorbate.

Embodiment 87. The method of embodiment 86, wherein the polysorbate is selected from the group consisting of polysorbate 20, polysorbate 40, polysorbate 60, and polysorbate 80.

Embodiment 88. A pharmaceutical composition obtained from the method of any one of embodiments 1-87.

Embodiment 89. A formulated antibody moiety composition comprising an antibody moiety and a polysorbate, wherein the composition has a reduced rate of polysorbate hydrolysis, wherein the shelf-life of the composition is more than 12 months.

Embodiment 90. A formulated antibody moiety composition comprising an antibody moiety and a polysorbate, wherein the composition has a reduced rate of polysorbate hydrolysis activity, wherein the shelf-life of the composition is extended compared to the shelf-life indicated in documents filed with a health authority related to the formulated antibody moiety composition, wherein the shelf-life is extended by at least 3 months compared to the shelf-life indicated in said documents.

Embodiment 91. The formulated antibody moiety composition of embodiment 89 or 90, wherein the rate of polysorbate hydrolysis is reduced by at least about 20%.

Embodiment 92. A formulated antibody moiety composition comprising an antibody moiety, wherein the formulated antibody moiety composition has a reduced degradation of polysorbate, wherein the degradation is reduced by at least about 20% compared to the degradation indicated in documents filed with a health authority related to the formulated antibody moiety composition.

Embodiment 93. A formulated antibody moiety composition comprising an antibody moiety and a polysorbate, wherein the polysorbate is degraded during storage of the liquid composition by 50% or less per year.

Embodiment 94. The formulated antibody moiety composition of any one of embodiments 89-93, wherein the antibody moiety is a monoclonal antibody.

Embodiment 95. The formulated antibody moiety composition of any one of embodiments 89-94, wherein the antibody moiety is a human, humanized, or chimeric antibody.

Embodiment 96. The formulated antibody moiety composition of any one of embodiments 89-95, wherein the antibody is selected from the group consisting of an anti-TAU antibody, an anti-TGFβ3 antibody, an anti-VEGF-A antibody, an anti-CD20 antibody, an anti-CD40 antibody, an anti-HER2 antibody, an anti-IL6 antibody, an anti-IgE antibody, an anti-IL13 antibody, an anti-TIGIT antibody, an anti-PD-L1 antibody, an anti-VEGF-A/ANG2 antibody, an anti-CD79b antibody, an anti-ST2 antibody, an anti-factor D antibody, an anti-factor IX antibody, an anti-factor X antibody, an anti-abeta antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, a FAP-IL2v fusion protein, and a TYRP1 TCB antibody.

Embodiment 97. The formulated antibody moiety composition of any one of embodiments 89-96, wherein the antibody moiety is selected from the group consisting of ocrelizumab, pertuzumab, ranibizumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, omalizumab ranibizumab, emicizumab, selicrelumab, prasinezumab, R06874281, and R07122290.

Embodiment 98. The formulated antibody moiety composition of any one of embodiments 89-97, wherein the polysorbate is selected from the group consisting of polysorbate 20, polysorbate 40, polysorbate 60, and polysorbate 80.

### EXAMPLES

### Example 1

This example demonstrates comparisons between the following purification platforms, the purification platforms comprising, in order, (1) a capture step, a CEX chromatography step comprising SP Sepharose® Fase Flow (SPSFF), an AEX chromatography step comprising Q Sepharose® Fast Flow (QSFF), a virus filtration step, and a UF/DF step (control purification platform); (2) a capture step, a CEX chromatography step comprising SP Sepharose® Fase Flow (SPSFF), an AEX chromatography step comprising Q Sepharose® Fast Flow (QSFF), a HIC step comprising Sartobind® Phenyl, a virus filtration step, and a UF/DF step; and (3) a capture step, a CEX chromatography step comprising SP Sepharose® Fase Flow (SPSFF), an AEX chromatography step comprising Q Sepharose® Fast Flow (QSFF), a depth filtration step comprising a X0SP depth filter, a virus filtration step, and a UF/DF step.

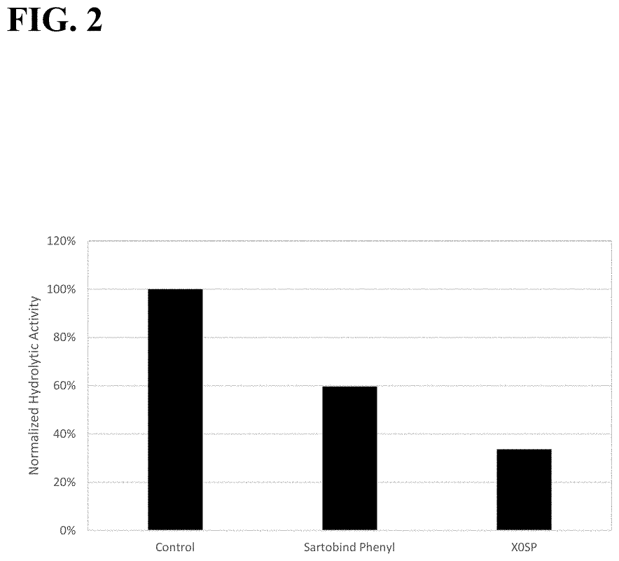

Hydrolytic activity of the resulting purified compositions was measured using a FAMS assay as described in the Materials and Methods section. As illustrated in FIG. 2, purification platforms (2), having a HIC step comprising Sartobind® Phenyl, and (3), having a depth filtration step comprising a X0SP depth filter, had reduced hydrolytic activity as compared to the control purification platform.

### Example 2

This example demonstrates comparisons between the use of three different HIC steps using the following purification platform comprising, in order: (1) a capture step, a Capto™ Adhere (MM-HIC/AEX) chromatography step, and a HIC step. The three different HIC medium tested were: Phenyl SFF HS, Toyopearl® Hexyl-650C, and Poros® Benzyl Ultra. Each HIC step was performed using a low-salt flow-through mode, wherein no HIC condition salts were added to the HIC load. The hydrolytic activity of a composition prior to a HIC step (HIC load) and following the HIC step (HIC pool) was measured. HIC steps were performed in a flow-through and low salt mode (no HIC conditioning salts were added to the HIC load). Operating conditions for the HIC step in flow-through and low salt mode are provided below in Table 1.

TABLE 1
HIC step operating conditions.

| Step | Solution |
|---|---|
| Equilibration | HIC Equilibration buffer, sodium acetate, pH 5-5.5 |
| Load | pH 5-5.5, no conductivity adjustment by addition of salt |
| Equilibration | Same as Equilibration buffer |
| Wash/Chase | |

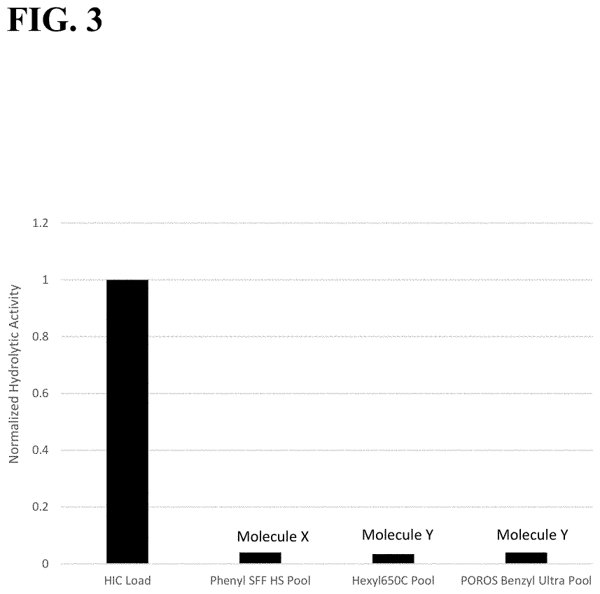

Hydrolytic activity of the HIC load and the HIC pool was measured using a FAMS assay as described in the Materials and Methods section. As illustrated in FIG. 3, the HIC step reduced the hydrolytic activity in the resulting HIC pool as compared to the starting material of the HIC load.

### Example 3

This example demonstrates the impact of including a MM-HIC/AEX chromatography step in a purification platform for the purification of two molecules, the purification platform comprising, in order: a capture step, a CEX step, e.g., Poros® 50HS, and, optionally, a MM-HIC/AEX chromatography step comprising Capto™ Adhere. The hydrolytic activity of a purified compositions without the MM-HIC/AEX chromatography step, namely, Capto™ Adhere (Capto™ Adhere load) and following the Capto™ Adhere chromatography step (Capto™ Adhere pool) were assessed. The Capto™ Adhere step was performed in flow-through mode and tests were performed at both pH 5.5 and 8. Operating conditions for MM-HIC/AEX in flow-through mode are provided below in Table 2.

TABLE 2
MM-HIC/AEX chromatography step operating conditions.

| Step | Solution |
|---|---|
| Equilibration | Equilibration buffer, either sodium acetate, |
| | pH 5.5 for low pI molecule or Tris, pH 8 for |
| | high pI molecule |
| Load | Adjust to pH 5.5 or pH 8 to match the equilibration buffer |
| | No conductivity adjustment by addition of salt |
| Equilibration | Same as Equilibration buffer |
| Wash/Chase | |

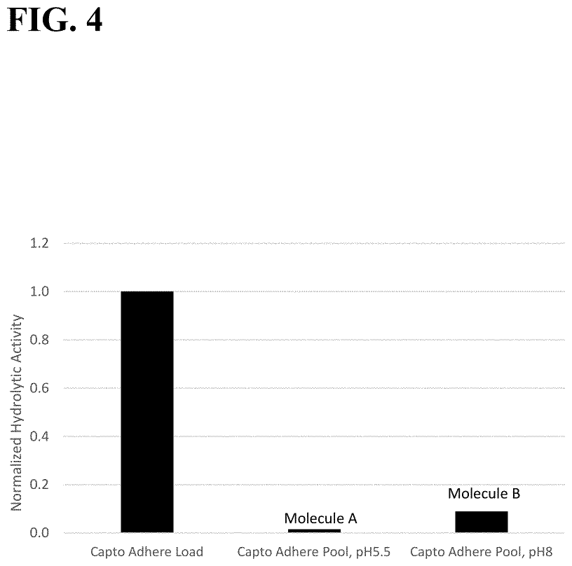

Hydrolytic activity of the Capto™ Adhere load and the Capto™ Adhere pool was measured using a FAMS assay as described in the Materials and Methods section. As illustrated in FIG. 4, the Capto™ Adhere chromatography step reduced the hydrolytic activity in the resulting Capto™ Adhere pools for each molecule purification as compared to the starting material of the Capto™ Adhere load. This reduction in hydrolytic activity was observed at both pH 5.5 (for molecule A) and 8 (for molecule B).

### Example 4

This example demonstrates comparisons between the following purification platforms, the purification platforms comprising, in order, (1) a CEX chromatography step comprising SP Sepharose® XL (SPXL), a HIC step comprising Bakerbond WP HI-Propyl™, a multimodal anion/cation exchange (MM-AEX/CEX) chromatography step comprising ABx™, an AEX chromatography step comprising Q Sepharose® Fast Flow (QSFF), and a UF/DF step; (2) a CEX chromatography step comprising SPXL, a HIC step comprising Bakerbond WP HI-Propyl™, a MM-AEX/CEX chromatography step comprising ABx™, an AEX chromatography step comprising QSFF, a depth filtration step comprising an EMPHAZE™ AEX depth filter, and a UF/DF step; (3) a CEX chromatography step comprising SPXL, a HIC step comprising Bakerbond WP HI-Propyl™, a MM-AEX/CEX chromatography step comprising ABx™, an AEX chromatography step comprising QSFF, a HIC step comprising Sartobind® Phenyl, and a UF/DF step; and (4) a CEX chromatography step comprising SPXL, a HIC step comprising Bakerbond WP HI-Propyl™, a MM-AEX/CEX chromatography step comprising ABx™, an AEX chromatography step comprising QSFF, a depth filtration step comprising a X0SP depth filter, and a UF/DF step.

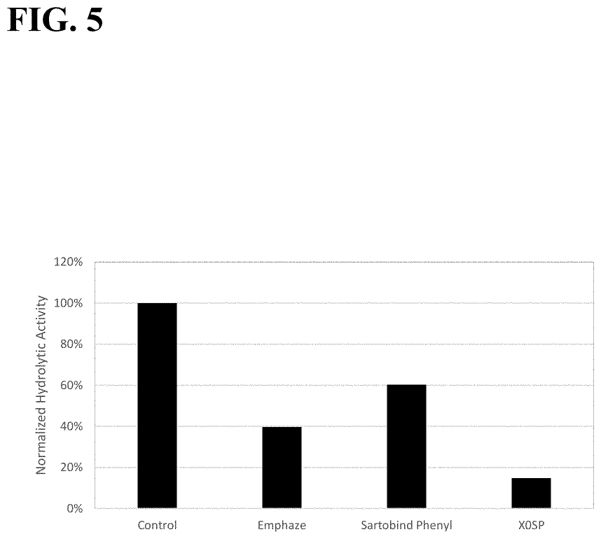

Hydrolytic activity of a composition from purification platform (1) and after the depth filtration step (for (2) and (4)) or after the Sartobind® Phenyl HIC step was measured from each platform using a FAMS assay as described in the Materials and Methods section. As illustrated in FIG. 5, purification platforms (2), having a depth filtration step comprising an EMPHAZE™ AEX depth filter, (3), having a HIC step comprising Sartobind® Phenyl, and (4), having a depth filtration step comprising a X0SP depth filter, had reduced hydrolytic activity as compared to the control purification platform (1).

### Materials and Methods

Determination of protein concentration. Protein concentrations were determined by UV spectroscopy either using a Cary® 50 UV-Vis Spectrophotometer (Varian) or NanoDrop™ OneC (Thermo Scientific). Protein samples were diluted in their respective buffers and measured as duplicates. Concentrations were determined according to the following equation deriving from Lambert-Beer law: c=(A 280 nm−A 320 nm)/ε·d·F with c protein concentration [mg/ml], A absorbance, F extinction coefficient [ml/(mg·cm)], d cell length [cm] and F dilution factor.

Hydrolytic activity assay (LEAP assay). The hydrolytic activity assay measured the hydrolytic activity by monitoring the conversion of a non-fluorescent substrate (4-MU, Chem Impex Int'l Inc) to a fluorescent product (MU, Sigma-Aldrich) through the cleavage of the substrate ester bond. Protein pool samples to be analyzed were rebuffered to 150 mM Tris-Cl pH 8.0 by using Amicon Ultra-0.5 ml centrifugal filter units (10,000 Da cut-off, Merck Millipore). The assay reaction mixture contained 80 μL of reaction buffer (150 mM Tris-Cl pH 8.0, 0.25% (w/v) Triton X-100 and 0.125% (w/v) Gum Arabic), 10 μL 4-MU substrate (1 mM in DMSO), and 10 μL protein pool sample. Protein pool sample concentrations were adjusted to 10-30 g/L and tested at three different concentrations. Each reaction was set up in three technical replicates in 96-well half-area polystyrene plates (black with lid and clear flat bottom, Corning Incorporated) and the increase of fluorescent signal (excitation at 355 nm, emission at 460 nm) was monitored every 10 min by incubating the reaction plate for two hours at 37° C. in an Infinite 200Pro plate reader (Tecan Life Sciences). MU production rate was derived from the slope of the fluorescent time course (0.5 hour-2 hour), and represents the raw rate of a reaction (k_{raw} [RFU/h]).

An enzyme blank reaction was additionally set up to measure any non-enzymatic cleavage of the substrate caused by the buffer matrix. 10 μL protein pool sample were replaced by 10 μL of 150 mM Tris-Cl pH 8.0 in the reaction mixture. The self-cleavage rate (k_{self}-cleavage [RFU/h]) was derived from the slope of the fluorescent time course (0.5 hour-2 hour). To convert the fluorescent signal (RFU) to μM of MU, a standard MU triplicate was added per plate. 10 μL MU (100 μM in DMSO) were supplemented with 10 μL of 150 mM Tris-Cl pH 8.0 and 80 μL of reaction buffer. The conversion factor a [RFU/μM] was calculated by averaging the fluorescent signal (0.5 hour-2 hours) and dividing it by the final concentration of MU present in the well.

The hydrolytic activity for a sample given in [μM MU/h] was determined by subtracting the reaction rate of the enzyme blank (k_{self}-cleavage [RFU/h]) from the reaction rate of the sample (k_{raw} [RFU/h]), and converting the fluorescent signal to μM MU/h by dividing the term by the conversion factor a [RFU/μM]. Activities were normalized to the protein concentration applied per well. To report hydrolytic activities in percent the hydrolytic activity of the reference sample was set to 100%.

Free fatty acid and mass spectrometry (FAMS) assay. To monitor the content of free fatty acids after PS20 degradation in the respective elution pools, samples were first prepared for PS20 stability studies and subsequently analyzed by mass spectrometry. Unless stated otherwise, protein pool samples were adjusted to the same protein concentration (as indicated in the respective experiment descriptions), containing 0.04% (w/v) SR-PS20, 10 mM L-Methionine and 100 mM Tris pH 8. L-methionine was added as an efficient antioxidant to control oxidative degradation of PS20 during the time-course of the experiment. As buffer control sample the applied protein volume was replaced by the same volume of the corresponding elution buffer system.

All reaction mixtures were incubated in a Thermomixer (Eppendorf) either at 25° C., or 40° C. Samples were withdrawn after defined time-points (as indicated in the respective figures, and stored at −80° C. until subsequent analysis.

50 μL of the sample was transferred to a new Eppendorf cup. 200 μL FFA dissolvent solution (500 ng/mL D23-lauric acid and 500 ng/mL ¹³C₁₄-myristic acid in acetonitrile) were added and vortexed briefly. The samples were centrifuged at 14.000 rpm for 5 minutes and transferred to an HPLC-vial for MS analysis. Separation of fatty acids from 5 μL of injected sample was performed on a Thermo Scientific™ Vanquish™ UHPLC-system using an ACQUITY UPLC® Peptide BEH C18 column (1.7 μm 2.1×150 mm and 300 Å). Eluent A (0.1% ammonium hydroxide in water) and Eluent B (100% acetonitrile) were used for the following gradient at a flow rate of 0.3 mL/min and a column temperature of 60° C. Initial conditions were at 70% eluent B. The gradient was changed linearly from 0.2 minute to 5.5 minute increasing eluent A to 100% and held until 6.0. Eluent B was set to 70% at 6.1 min and held until 10.0 min for equilibration. The Mass spectrometer (Triple TOF® 6600, AB Sciex) was operated in negative ionization mode with ion spray voltage at −4500 V. Source temperature was set to 450° C. and TOF mass range was 100-1000 m/Z. Declustering potential was −120 V and collision energy −10 V.

XICs for the masses of lauric acid, myristic acid, and isotopically labelled (D23)-lauric acid and (¹³C₁₄) myristic acid were generated. Respective peaks were integrated and the peak area ratio between lauric acid and D23-lauric acid as well as the ratio between myristic acid and ¹³C₁₄-myristic acid were determined. The peak area ratio was used to calculate the concentrations of lauric acid and myristic acid in the samples. Measurements were performed in duplicate. To report amount FFA (lauric acid (LA) and myristic acid (MA)) in percent the amount of the reference sample was set to 100%.

### Example 5

This example demonstrates comparisons between the following purification platforms for purifying Molecule D, the purification platforms comprising, in order, (1) a capture step, a CEX chromatography step comprising SP Sepharose® Fase Flow (SPSFF), an AEX chromatography step comprising Q Sepharose® Fast Flow (QSFF), a virus filtration step, and a UF/DF step (control purification platform); and (2) a capture step, a CEX chromatography step comprising SP Sepharose® Fase Flow (SPSFF), an AEX chromatography step comprising Q Sepharose® Fast Flow (QSFF), a depth filtration step comprising a Polisher ST depth filter, a virus filtration step, and a UF/DF step. For the purification platform (2), the depth filtration step was performed at pH 6.

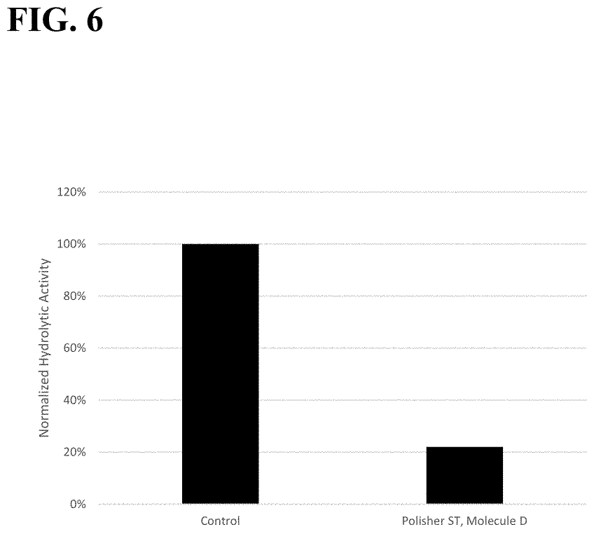

Hydrolytic activity of the resulting purified compositions was measured using a FAMS assay as described in the Materials and Methods section of Example 4. As illustrated in FIG. 6, purification platform (2), having a depth filtration step comprising a Polisher ST depth filter, provided a resulting composition having reduced hydrolytic activity as compared to the control purification platform without the Polisher ST depth filtration step.

### Example 6

This example demonstrates comparisons between the following purification platforms for purifying Molecule E, the purification platforms comprising, in order, (1) a capture step, a CEX step, e.g., Poros® 50HS, and a MM-HIC/AEX chromatography step comprising Capto™ Adhere (control purification platform); and (2) a capture step, a CEX step, e.g., Poros® 50HS, and a depth filtration step comprising a Polisher ST depth filter to filter the load for a MM-HIC/AEX chromatography step comprising Capto™ Adhere. For the purification platform (2), the depth filtration step was performed at pH 8.

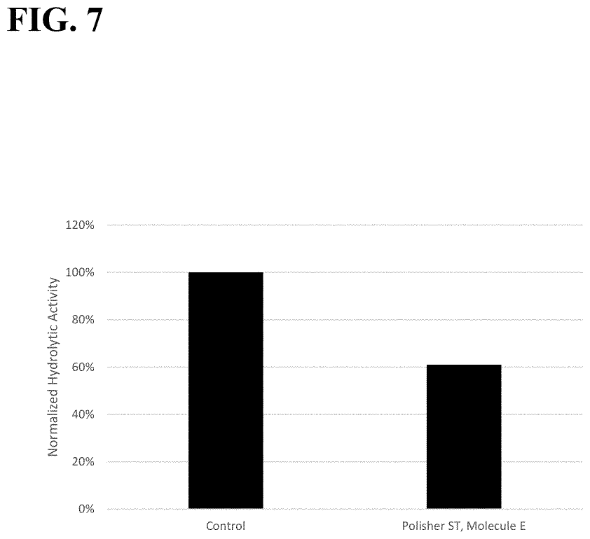

Hydrolytic activity of the resulting purified compositions was measured using a FAMS assay as described in the Materials and Methods section of Example 4. As illustrated in FIG. 7, purification platform (2), having a depth filtration step comprising a Polisher ST depth filter, provided a resulting composition having reduced hydrolytic activity as compared to the control purification platform without the Polisher ST depth filtration step.

What is claimed is:

1. A method of reducing a hydrolytic enzyme activity rate of a composition obtained from a purification platform, the method comprising subjecting a sample to the purification platform comprising:
a capture step;
one or more ion exchange (IEX) chromatography steps; and
a depth filtration step,
thereby reducing the hydrolytic enzyme activity rate of the composition as compared to purification of the sample without the depth filtration step.

2. The method of claim 1, wherein each of the one or more IEX chromatography steps is selected from the group consisting of: an anion exchange (AEX) chromatography step, a cation exchange (CEX) chromatography step, and a multimodal ion exchange (MMIEX) chromatography step.

3. The method of claim 2, wherein the MMIEX chromatography step comprises a multimodal cation exchange/anion exchange (MM-AEX/CEX) chromatography step.

4. The method of any one of claims 1-3, further comprising a virus filtration step.

5. The method of any one of claims 1-4, further comprising an ultrafiltration/diafiltration (UF/DF) step.

6. The method of claim 5, wherein the purification platform comprises, in order:
the capture step;
the CEX chromatography step;
the AEX chromatography step;
the depth filtration step;
the virus filtration step; and
the UF/DF step.

7. The method of any one of claims 1-6, wherein the depth filtration step comprises processing via a depth filter, and wherein the depth filter is a X0SP depth filter, a C0SP depth filter, a D0SP depth filter, a Polisher ST depth filter, or an EMPHAZE™ depth filter.

8. The method of any one of claims 1-6, further comprising a HIC step comprising processing via Sartobind® phenyl.

9. A method of reducing a hydrolytic enzyme activity rate of a composition obtained from a purification platform, the method comprising subjecting a sample to the purification platform comprising:
a capture step;
a multimodal hydrophobic interaction/ion exchange (MM-HIC/IEX) chromatography step; and
a hydrophobic interaction chromatography (HIC) step,
thereby reducing the hydrolytic enzyme activity rate of the composition as compared to purification of the sample without the HIC step and/or the MM-HIC/IEX chromatography step.

10. The method of claim 9, wherein the MM-HIC/IEX chromatography step comprises processing via a MM-HIC/IEX chromatography medium and the processing is performed at a pH of about 4.5 to about 9.

11. The method of claim 9 or 10, wherein the MM-HIC/IEX chromatography step is a multimodal hydrophobic interaction/anion exchange (MM-HIC/AEX) chromatography step.

12. The method of claim 11, wherein the MM-HIC/AEX chromatography step comprises processing via Capto™ Adhere or Capto™ Adhere ImpRes.

13. The method of claim 9 or 10, wherein the MM-HIC/IEX chromatography step is a multimodal hydrophobic interaction/cation exchange (MM-HIC/CEX) chromatography step.

14. The method of claim 13, wherein the MM-HIC/CEX chromatography step comprises Capto™ MMC or Capto™ MMC ImpRes.

15. The method of any one of claims 9-14, wherein the purification platform comprises, in order:
the capture step;
the MM-HIC/IEX chromatography step; and
the HIC step.

16. The method of any one of claims 9-15, further comprising a virus filtration step.

17. The method of any one of claims 9-16, further comprising an ultrafiltration/diafiltration (UF/DF) step.

18. The method of claim 17, wherein the purification platform comprises, in order:
the capture step;
the MM-HIC/AEX chromatography step;
the HIC step;
the virus filtration step; and
the UF/DF step.

19. The method of any one of claims 9-18, further comprising a depth filtration step.

20. The method of claim 19, wherein the purification platform comprises, in order:
the capture step;
the depth filtration step;
the MM-HIC/AEX chromatography step; and
the HIC step.

21. The method of claim 19 or 20, wherein the depth filtration step comprises processing via a depth filter, and wherein the depth filter is a X0SP depth filter.

22. The method of claim 19 or 20, wherein the depth filtration step comprises processing via a depth filter, and the depth filter is an EMPHAZE™ depth filter or a Polisher ST depth filter.

23. The method of any one of claims 19-22, wherein the depth filter is used as a load filter in conjunction with the MM-HIC/AEX chromatography step.

24. The method of claim 23, wherein the MM-HIC/AEX chromatography step comprises processing via Capto™ Adhere or Capto™ Adhere ImpRes.

25. The method of claim 19, wherein the purification platform comprises, in order:
the capture step;
the MM-HIC/AEX chromatography step;
the depth filtration step; and
the HIC step.

26. The method of claim 25, wherein the depth filtration step comprises processing via a depth filter, and wherein the depth filter is a X0SP depth filter, a C0SP depth filter, or a D0SP depth filter.

27. The method of claim 25, wherein the depth filtration step comprises processing via a depth filter, and the depth filter is an EMPHAZE™ depth filter or a Polisher ST depth filter.

28. The method of claim any one of claims 25-27, wherein the MM-HIC/AEX chromatography step comprises processing via Capto™ Adhere or Capto™ Adhere ImpRes.

29. A method of reducing a hydrolytic enzyme activity rate of a composition obtained from a purification platform, the method comprising subjecting a sample to the purification platform comprising:
a capture step;
one or more ion exchange (IEX) chromatography steps; and
a hydrophobic interaction chromatography (HIC) step,
thereby reducing the hydrolytic enzyme activity rate of the composition as compared to purification of the sample without the HIC step.

30. The method of claim 29, wherein the one or more IEX chromatography steps is a cation exchange (CEX) chromatography step.

31. The method of claim 29 or 30, further comprising a virus filtration step.

32. The method of any one of claims 29-31, further comprising an ultrafiltration/diafiltration (UF/DF) step.

33. The method of claim 32, further comprising a depth filtration step performed at any stage prior to the UF/DF step.

34. The method of claim 33, wherein the purification platform comprises, in order:
the capture step;
the CEX chromatography step;
the HIC step;
the virus filtration step; and
the UF/DF step.

35. A method of reducing a hydrolytic enzyme activity rate of a composition obtained from a purification platform, the method comprising subjecting a sample to the purification platform comprising:
one or more ion exchange (IEX) chromatography steps;
a hydrophobic interaction chromatography (HIC) step; and
a depth filtration step,
thereby reducing the hydrolytic enzyme activity rate of the composition as compared to purification of the sample without the HIC step or the depth filtration step.

36. The method of claim 35, wherein the reduction is as compared to purification of the sample without the HIC and the depth filtration step.

37. The method of claim 35 or 36, wherein each of the one or more IEX chromatography steps is selected from the group consisting of: an anion exchange (AEX) chromatography step, a cation exchange (CEX) chromatography step, and a multimodal ion exchange (MMIEX) chromatography step.

38. The method of claim 37, wherein the MMIEX chromatography step comprises a multimodal cation exchange/anion exchange (MM-AEX/CEX) chromatography step.

39. The method of any one of claims 35-38, further comprising an ultrafiltration/diafiltration (UF/DF) step.

40. The method of claim 39, wherein the purification platform comprises, in order:
the CEX chromatography step;
the HIC step;
the MMIEX chromatography step;
the AEX chromatography step;
the depth filter step; and
the UF/DF step.

41. A method of reducing a hydrolytic enzyme activity rate of a composition obtained from a purification platform, the method comprising subjecting a sample to the purification platform comprising:
a capture step;
one or more ion exchange (IEX) chromatography steps;
a multimodal hydrophobic interaction/ion exchange (MM-HIC/IEX) chromatography steps; and
one or both of:
a hydrophobic interaction chromatography (HIC) step; and
a depth filtration step,
thereby reducing the hydrolytic enzyme activity rate of the composition as compared to purification of the sample without the HIC step or the depth filtration step.

42. The method of claim 41, wherein the reduction is as compared to purification of the sample without the HIC and the depth filtration step.

43. The method of claim 41 or 42, further comprising a virus filtration step.

44. The method of any one of claims 41-43, further comprising an ultrafiltration/diafiltration (UF/DF) step.

45. The method of any one of claims 41-44, wherein the depth filtration step is performed as a load filter for the MM-HIC/IEX chromatography step, as a load filter for the HIC step, or following the HIC step.

46. The method of any one of claim 41-45, wherein each of the one or more IEX chromatography steps is selected from the group consisting of: a cation exchange (CEX) chromatography step, an anion exchange (AEX) chromatography step, and a multimodal ion exchange (MMIEX) chromatography step.

47. The method of any one of claims 41-46, wherein the MM-HIC/IEX chromatography step is a multimodal hydrophobic interaction/anion exchange (MM-HIC/AEX) chromatography step.

48. The method of claim 47, wherein the MM-HIC/AEX chromatography step comprises processing via Capto™ Adhere or Capto™ Adhere ImpRes.

49. The method of any one of claims 1-34 and 41-48, wherein the capture step comprises processing via affinity chromatography.

50. The method of any one of claims 1-34 and 41-48, wherein the capture step is performed in a bind-and-elute mode.

51. The method of claim 49 or 50, wherein the affinity chromatography is selected from the group consisting of a protein A chromatography, a protein G chromatography, a protein A/G chromatography, a FcXL chromatography, a protein XL chromatography, a kappa chromatography, and a kappaXL chromatography.

52. The method of any one of claims 1-6, 19, 20, and 35-51, wherein the depth filtration step comprises processing via a depth filter.

53. The method of claim 52, wherein the depth filter is used as a load filter.

54. The method of claim 52 or 53, wherein the depth filter comprises a substrate comprising one or more of a diatomaceous earth composition, a silica composition, a cellulose fiber, a polymeric fiber, a cohesive resin, and an ash composition.

55. The method of claim 54, wherein at least a portion of the substrate of the depth filter comprises a surface modification.

56. The method of claim 55, wherein the surface modification is one or more of a quaternary amine surface modification, a cationic surface modification, and an anionic surface modification.

57. The method of any one of claims 52-56, wherein the depth filter is selected from the group consisting of: a X0SP depth filter, a D0SP depth filter, a C0SP depth filter, an EMPHAZE™ depth filter, a PDD1 depth filter, a PDE1 depth filter, a PDH5 depth filter, a ZETA PLUS™ 120ZA depth filter, a ZETA PLUS™ 120ZB depth filter, a ZETA PLUS™ DELI depth filter, a ZETA PLUS™ DELP depth filter, and a Polisher ST depth filter.

58. The method of claim 57, wherein the depth filter is the X0SP depth filter, the D0SP depth filter, or the C0SP depth filter, and wherein the processing via the depth filter is performed at a pH of about 4.5 to about 8.

59. The method of claim 57, wherein the depth filter is the EMPHAZE™ depth filter, and wherein processing via the depth filter is performed at a pH of about 7 to about 9.5.

60. The method of claim 57, wherein the depth filter is the Polisher ST depth filter, and wherein processing via the depth filter is performed at a pH of about 4.5 to about 9.

61. The method of any one of claims 9-60, wherein the HIC step comprises processing via a HIC membrane or a HIC column.

62. The method of claim 61, wherein processing via the HIC membrane or the HIC column is performed using low salt concentrations.

63. The method of any one of claims 59-61, wherein processing via the HIC membrane or the HIC column is performed in flow-through mode.

64. The method of any one of claims 61-63, wherein the HIC membrane or HIC column comprises a substrate comprising one or more of an ether group, an ethyl group, a propyl group, an isopropyl group, a butyl group, a hexyl group, an octyl group, and a phenyl group.

65. The method of any one of claims 61-64, wherein the HIC membrane or the HIC column is selected from the group consisting of Bakerbond WP HI-Propyl™, Phenyl Sepharose® Fast Flow (Phenyl-SFF), Phenyl Sepharose® Fast Flow Hi-sub (Phenyl-SFF HS), Toyopearl® Hexyl-650C, Toyopearl® Hexyl-650M, Toyopearl® Hexyl-650S, Poros™ Benzyl Ultra, and Sartobind® phenyl.

66. The method of claim 64 or 65, wherein processing via the HIC membrane or the HIC column is performed at a pH of about 4.5 to about 7.

67. The method of any one of claims 1-8 and 29-66, wherein each of the one or more IEX chromatography steps comprises processing via an IEX chromatography membrane or an IEX chromatography column.

68. The method of claim 67, wherein the IEX chromatography membrane or the IEX chromatography column is selected from the group consisting of: SPSFF, QSFF, SPXL, Streamline™ SPXL, ABx™, Poros™ XS, Poros™ 50HS, DEAE, DMAE, TMAE, QAE, and MEP-Hypercel™.

69. The method of any one of claims 1-68, wherein the purification platform is for purification of a target from the sample, and wherein the sample comprises the target and one or more host cell impurities.

70. The method of claim 69, wherein the target comprises a polypeptide.

71. The method of any one of claims 1-70, wherein the target is an antibody moiety.

72. The method of claim 71, wherein the antibody moiety is a monoclonal antibody.

73. The method of claim 71 or 72, wherein the antibody moiety is a human, humanized, or chimeric antibody.

74. The method of any one of claims 71-73, wherein the antibody moiety is selected from the group consisting of: an anti-TAU antibody, an anti-TGFβ3 antibody, an anti-VEGF-A antibody, an anti-CD20 antibody, an anti-CD40 antibody, an anti-HER2 antibody, an anti-IL6 antibody, an anti-IgE antibody, an anti-IL13 antibody, an anti-TIGIT antibody, an anti-PD-L1 antibody, an anti-VEGF-A/ANG2 antibody, an anti-CD79b antibody, an anti-ST2 antibody, an anti-factor D antibody, an anti-factor IX antibody, an anti-factor X antibody, an anti-abeta antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, a FAP-IL2v fusion protein, and a TYRP1 TCB antibody.

75. The method of any one of claims 71-74, wherein the antibody moiety is selected from the group consisting of: ocrelizumab, pertuzumab, ranibizumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, omalizumab, ranibizumab, emicizumab, selicrelumab, prasinezumab, RO6874281, and RO7122290.

76. The method of any one of claims 69-75, wherein the one or more host cell impurities comprises a host cell protein.

77. The method of claim 76, wherein the host cell protein is a hydrolytic enzyme.

78. The method of claim 77, wherein the hydrolytic enzyme is a lipase, an esterase, a thioesterase, a phospholipase, carboxylesterase, hydrolase, cutinase, or a ceramidase.

79. The method of any one of claims 1-78, wherein the sample comprises a host cell or components originating therefrom.

80. The method of any one of claims 1-79, wherein the sample is, or is derived from, a cell culture sample.

81. The method of claim 80, wherein the cell culture sample comprises a host cell, and wherein the host cell is a Chinese hamster ovary (CHO) cell or an E. coli cell.

82. The method of any one of claims 1-81, further comprising a sample processing step.

83. The method of any one of claims 1-82, wherein the reduction in the hydrolytic enzyme activity rate is at least about 20%.

84. The method of any one of claims 1-83, further comprising determining the hydrolytic enzyme activity rate of the composition.

85. The method of any one of claims 1-84, further comprising determining the level of one or more hydrolytic enzymes in the composition.

86. The method of any one of claims 1-85, wherein the composition comprises a polysorbate.

87. The method of claim 86, wherein the polysorbate is selected from the group consisting of polysorbate 20, polysorbate 40, polysorbate 60, and polysorbate 80.

88. A pharmaceutical composition obtained from the method of any one of claims 1-87.

89. A formulated antibody moiety composition comprising an antibody moiety and a polysorbate, wherein the composition has a reduced rate of polysorbate hydrolysis, wherein the shelf-life of the composition is more than 12 months.

90. A formulated antibody moiety composition comprising an antibody moiety and a polysorbate, wherein the composition has a reduced rate of polysorbate hydrolysis activity, wherein the shelf-life of the composition is extended compared to the shelf-life indicated in documents filed with a health authority related to the formulated antibody moiety composition, wherein the shelf-life is extended by at least 3 months compared to the shelf-life indicated in said documents.

91. The formulated antibody moiety composition of claim 89 or 90, wherein the rate of polysorbate hydrolysis is reduced by at least about 20%.

92. A formulated antibody moiety composition comprising an antibody moiety, wherein the formulated antibody moiety composition has a reduced degradation of polysorbate, wherein the degradation is reduced by at least about 20% compared to the degradation indicated in documents filed with a health authority related to the formulated antibody moiety composition.

93. A formulated antibody moiety composition comprising an antibody moiety and a polysorbate, wherein the polysorbate is degraded during storage of the liquid composition by 50% or less per year.

94. The formulated antibody moiety composition of any one of claims 89-93, wherein the antibody moiety is a monoclonal antibody.

95. The formulated antibody moiety composition of any one of claims 89-94, wherein the antibody moiety is a human, humanized, or chimeric antibody.

96. The formulated antibody moiety composition of any one of claims 89-95, wherein the antibody is selected from the group consisting of an anti-TAU antibody, an anti-TGFβ3 antibody, an anti-VEGF-A antibody, an anti-CD20 antibody, an anti-CD40 antibody, an anti-HER2 antibody, an anti-IL6 antibody, an anti-IgE antibody, an anti-IL13 antibody, an anti-TIGIT antibody, an anti-PD-L1 antibody, an anti-VEGF-A/ANG2 antibody, an anti-CD79b antibody, an anti-ST2 antibody, an anti-factor D antibody, an anti-factor IX antibody, an anti-factor X antibody, an anti-abeta antibody, an anti-CEA antibody, an anti-CEA/CD3 antibody, an anti-CD20/CD3 antibody, an anti-FcRH5/CD3 antibody, an anti-Her2/CD3 antibody, an anti-FGFR1/KLB antibody, a FAP-4-1 BBL fusion protein, a FAP-IL2v fusion protein, and a TYRP1 TCB antibody.

97. The formulated antibody moiety composition of any one of claims 89-96, wherein the antibody moiety is selected from the group consisting of ocrelizumab, pertuzumab, ranibizumab, trastuzumab, tocilizumab, faricimab, polatuzumab, gantenerumab, cibisatamab, crenezumab, mosunetuzumab, tiragolumab, bevacizumab, rituximab, atezolizumab, obinutuzumab, lampalizumab, omalizumab ranibizumab, emicizumab, selicrelumab, prasinezumab, R06874281, and R07122290.

98. The formulated antibody moiety composition of any one of claims 89-97, wherein the polysorbate is selected from the group consisting of polysorbate 20, polysorbate 40, polysorbate 60, and polysorbate 80.
